SEQUENCE LISTING (29 sequences)

SEQ ID NO: 1 - DNA, 56 bp, Artificial Sequence
  Synthetic
gaaattaatacgactcactataggcattgcagagtttcttcagttaggtctaagcc

SEQ ID NO: 2 - DNA, 32 bp, Artificial Sequence
  Synthetic
aatttttaagaaaagagcccttggtttggata

SEQ ID NO: 3 - DNA, 60 bp, Artificial Sequence
  Synthetic
gaaattaatacgactcactatagggcaccagccgaacgtggtgatccgccgatcgatgac

SEQ ID NO: 4 - DNA, 33 bp, Artificial Sequence
  Synthetic
ctatcagcaactggaccatggccagaaaaatcg

SEQ ID NO: 5 - DNA, 56 bp, Artificial Sequence
  Synthetic
gaaattaatacgactcactataggtatccacctacaatccttgaaagaccttaaac

SEQ ID NO: 6 - DNA, 32 bp, Artificial Sequence
  Synthetic
ttagacatgtttgaactccattcttcagtgta

SEQ ID NO: 7 - DNA, 22 bp, Artificial Sequence
  Synthetic
cttttcctcttgggcatcatct

SEQ ID NO: 8 - DNA, 21 bp, Artificial Sequence
  Synthetic
aggaacagcgaccctttctca

SEQ ID NO: 9 - DNA, 28 bp, Artificial Sequence
  Synthetic
  modified_base (1)..(1): A is modified with fluorescein
  modified_base (28)..(28): A is modified with TAMRA
tactggggttccttgcactccaatcaga

SEQ ID NO: 10 - DNA, 20 bp, Artificial Sequence
  Synthetic
gcccgaagcgtttactttga

SEQ ID NO: 11 - DNA, 24 bp, Artificial Sequence
  Synthetic
tccattattcctagctgcggtatc

SEQ ID NO: 12 - DNA, 19 bp, Artificial Sequence
  Synthetic
  modified_base (1)..(1): A is modified with fluorescein
  modified_base (19)..(19): C is modified with TAMRA
aaagcaggcccgagccgcc

SEQ ID NO: 13 - DNA, 24 bp, Artificial Sequence
  Synthetic
gaaattaatacgactcactatagg

SEQ ID NO: 14 - DNA, 88 bp, Artificial Sequence
  Synthetic
ctgtgtgaagcagtcaatgcagtagcaagttttagtccccttcgtttttggggtagtctaaatccctatagtgagtcgtattaatttc

SEQ ID NO: 15 - DNA, 88 bp, Artificial Sequence
  Synthetic
cgcgtcagcggatccacacggacctcgtgttttagtccccttcgtttttggggtagtctaaatccctatagtgagtcgtattaatttc

SEQ ID NO: 16 - DNA, 88 bp, Artificial Sequence
  Synthetic
gcccttcctgcgagaaaattgaaatcatgttttagtccccttcgtttttggggtagtctaaatccctatagtgagtcgtattaatttc

SEQ ID NO: 17 - DNA, 64 bp, Artificial Sequence
  Synthetic
gatttagactaccccaaaaacgaaggggactaaaacttgctactgcattgactgcttcacacag

SEQ ID NO: 18 - DNA, 64 bp, Artificial Sequence
  Synthetic
gatttagactaccccaaaaacgaaggggactaaaacacgaggtccgtgtggatccgctgacgcg

SEQ ID NO: 19 - DNA, 64 bp, Artificial Sequence
  Synthetic
gatttagactaccccaaaaacgaaggggactaaaacatgatttcaattttctcgcaggaagggc

SEQ ID NO: 20 - DNA, 28 bp, Artificial Sequence
  Synthetic
ttgctactgcattgactgcttcacacag

SEQ ID NO: 21 - DNA, 28 bp, Artificial Sequence
  Synthetic
acgaggtccgtgtggatccgctgacgcg

SEQ ID NO: 22 - DNA, 28 bp, Artificial Sequence
  Synthetic
atgatttcaattttctcgcaggaagggc

SEQ ID NO: 23 - DNA, 402 bp, Artificial Sequence
  Synthetic
gaaattaatacgactcactataggatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 24 - RNA, 14 nt, Artificial Sequence
  Synthetic
  modified_base (1)..(1): A is modified with fluorescein
  modified_base (1)..(1): A is 2′-O-methyl A
  modified_base (2)..(2): A is riboA
  modified_base (3)..(3): U is riboU
  modified_base (4)..(5): G is riboG
  modified_base (6)..(6): C is riboC
  modified_base (7)..(8): A is 2′-O-methyl A
  modified_base (9)..(9): A is riboA
  modified_base (10)..(10): U is riboU
  modified_base (11)..(12): G is riboG
  modified_base (13)..(13): C is riboC
  modified_base (14)..(14): A is modified with biotin
  modified_base (14)..(14): A is 2′-O-methyl A
aauggcaaauggca

SEQ ID NO: 25 - DNA, 32 bp, Artificial Sequence
  Synthetic
cattgcagagtttcttcagttaggtctaagcc

SEQ ID NO: 26 - DNA, 36 bp, Artificial Sequence
  Synthetic
gcaccagccgaacgtggtgatccgccgatcgatgac

SEQ ID NO: 27 - DNA, 32 bp, Artificial Sequence
  Synthetic
tatccacctacaatccttgaaagaccttaaac

SEQ ID NO: 28 - DNA, 378 bp, Artificial Sequence
  Synthetic
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 29 - protein, 1182 aa, Artificial Sequence
  Synthetic
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

(19) United States
(12) Patent Application Publication
(10) Pub. No.: US 2022/0282341 A1
(43) Pub. Date: Sep. 8, 2022

(54) TRANSPLANT DIAGNOSTICS USING CRISPR-BASED TECHNOLOGY

(71) Applicants: Massachusetts Institute of Technology, Cambridge, MA (US); The Brigham and Women's Hospital, Inc., Boston, MA (US)

(72) Inventors: Leonardo V. Riella, Newton, MA (US); James J. Collins, Newton, MA (US); Michael M. Kaminski, Berlin (DE)

(73) Assignees: Massachusetts Institute of Technology, Cambridge, MA (US); The Brigham and Women's Hospital, Inc., Boston, MA (US)

(21) Appl. No.: 17/632,733

(22) PCT Filed: Aug. 5, 2020
(86) PCT No.: PCT/US2020/044998

§ 371 Date: Feb. 3, 2022

Related U.S. Application Data
(60) Provisional application No. 62/882,652, filed on Aug. 5, 2019.

(51) Int. Cl. C12Q 1/70; C12Q 1/6883; B01L 3/00

(52) U.S. Cl. CPC C12Q 1/701; C12Q 1/6883; B01L 3/5023; C12Q 1/6823

(57) ABSTRACT

Described herein are nucleic acid detection systems, devices, and kits having a CRISPR component comprising an effector protein and a guide RNA, and/or a polynucleotide encoding a guide RNA, that binds or hybridizes to a corresponding target molecule. Also described herein are methods that utilize these nucleic acid systems, devices, and kits.

### RELATED APPLICATION

This application claims priority under 35 U.S.C. § 119(e) to U.S. provisional application No. 62/882,652, filed Aug. 5, 2019, which is incorporated herein by reference in its entirety.

### FIELD

Described herein are nucleic acid detection systems, devices, and kits having a CRISPR component comprising an effector protein and a guide RNA, and/or a polynucleotide encoding a guide RNA, that binds or hybridizes to a corresponding target molecule. Also described herein are methods that utilize these nucleic acid systems, devices, and kits.

### BACKGROUND

The CRISPR and Cas immune system has recently been adapted for the detection of nucleic acids (14-17, 56-59). These protocols enable rapid, cost-effective DNA and RNA detection in a variety of sample types with excellent sensitivity and specificity, making them ideal tools for point-of-care (POC) testing. However, most studies to date have used synthetic standards, include few clinical specimens, and lack direct comparison to clinical gold-standard diagnostics.

### SUMMARY

Fast and cost-effective POC testing should enable early diagnosis and greater accessibility for patients in low-resource settings, including the opportunity for self-monitoring. Here, CRISPR-Cas13 diagnostics (SHERLOCK, specific high-sensitivity enzymatic reporter unlocking) were applied to detect infection with CMV and BKV in samples from recipients of kidney transplants. The use of SHERLOCK was extended for the detection of CXCL9 mRNA, a biomarker of acute cellular rejection of kidney transplants. Together, these developments enable the cost-effective monitoring of patients at risk of opportunistic infection and serve as a tool for earlier detection of rejection and monitoring after organ transplantation.

Accordingly, in some aspects, the disclosure relates to nucleic acid detection systems. In some embodiments, a nucleic acid detection system comprises: a CRISPR component comprising: (i) an effector protein; and (ii) a guide RNA, and/or a polynucleotide encoding a guide RNA, that binds or hybridizes to a corresponding target molecule; wherein the target molecule polynucleic acid is indicative of rejection and/or infection of an organ transplant.

In some embodiments, the effector protein is Cas13.

In some embodiments, the target molecule is selected from the group consisting of viral DNA and cytokine mRNA. In some embodiments, the target molecule is selected from the group consisting of BK polyomavirus DNA, cytomegalovirus DNA, CXCL9 mRNA, CXCL10 mRNA, and a combination thereof.

In some embodiments, a detection system comprises: a guide RNA comprising the nucleic acid sequence of TTGCTACTGCATTGACTGCTTCACACAG (SEQ ID NO: 20); a guide RNA comprising the nucleic acid sequence of ACGAGGTCCGTGTGGATCCGCTGACGCG (SEQ ID NO: 21); a guide RNA comprising the nucleic acid sequence of ATGATTTCAATTTTCTCGCAGGAAGGGC (SEQ ID NO: 22); or a combination thereof.

In some embodiments, a detection system comprises: a guide RNA comprising the nucleic acid sequence of GATTTAGACTACCCCAAAAACGAAGGGGACTAAAACTTGCTACTGCATTGACTG CTTCACACAG (SEQ ID NO: 17); a guide RNA comprising the nucleic acid sequence of GATTTAGACTACCCCAAAAACGAAGGGGACTAAAACACGAGGTCCGTGTGGATC CGCTGACGCG (SEQ ID NO: 18); a guide RNA comprising the nucleic acid sequence of GATTTAGACTACCCCAAAAACGAAGGGGACTAAAACATGATTTCAATTTTCTCGC AGGAAGGGC (SEQ ID NO: 19); or a combination thereof.

In some embodiments, a detection system comprises: a polynucleotide encoding a guide RNA and comprising the nucleic acid sequence of CTGTGTGAAGCAGTCAATGCAGTAGCAAGTTTTAGTCCCCTTCGTTTTTGGGGTA GTCTAAATCCCTATAGTGAGTCGTATTAATTTC (SEQ ID NO: 14); a polynucleotide encoding a guide RNA and comprising the nucleic acid sequence of CGCGTCAGCGGATCCACACGGACCTCGTGTTTTAGTCCCCTTCGTTTTTGGGGTA GTCTAAATCCCTATAGTGAGTCGTATTAATTTC (SEQ ID NO: 15); a polynucleotide encoding a guide RNA and comprising the nucleic acid sequence of GCCCTTCCTGCGAGAAAATTGAAATCATGTTTTAGTCCCCTTCGTTTTTGGGGTAG TCTAAATCCCTATAGTGAGTCGTATTAATTTC (SEQ ID NO: 16); or a combination thereof.

In some embodiments, a detection system further comprises an amplification component, wherein the amplification component comprises a polymerase and one or more primer.

In some embodiments, an amplification component comprises a DNA polymerase, an RNA polymerase, or a combination thereof. In some embodiments, the amplification component comprises an RNA polymerase, wherein the RNA polymerase is T7 RNA polymerase.

In some embodiments, a detection system comprises a forward primer and a reverse primer, wherein the forward and reverse primer concentrations are 120 nM and 480 nM, respectively.

In some embodiments, a detection system comprises a forward primer and a reverse primer, wherein the reverse primer comprises an RNA polymerase promoter sequence. In some embodiments, the RNA polymerase promoter sequence is a T7 polymerase promoter sequence. In some embodiments, the T7 polymerase promoter sequence comprises the nucleic acid sequence of GAAATTAATACGACTCACTATAGG (SEQ ID NO: 13).

In some embodiments, a detection system comprises: a forward primer comprising the nucleic acid sequence of CATTGCAGAGTTTCTTCAGTTAGGTCTAAGCC (SEQ ID NO: 25); and a reverse primer comprising the nucleic acid sequence of AATTTTTAAGAAAAGAGCCCTTGGTTTGGATA (SEQ ID NO: 2). In some embodiments, the forward primer comprises the nucleic acid sequence of GAAATTAATACGACTCACTATAGGCATTGCAGAGTTTCTTCAGTTAGGTCTAAGC C (SEQ ID NO: 1).

In some embodiments, a detection system comprises: a forward primer comprising the nucleic acid sequence of GCACCAGCCGAACGTGGTGATCCGCCGATCGATGAC (SEQ ID NO: 26); and a reverse primer comprising the nucleic acid sequence of CTATCAGCAACTGGACCATGGCCAGAAAAATCG (SEQ ID NO: 4). In some embodiments, the forward primer comprises the nucleic acid sequence of GAAATTAATACGACTCACTATAGGGCACCAGCCGAACGTGGTGATCCGCCGATC GATGAC (SEQ ID NO: 3).

In some embodiments, a detection system comprises: a forward primer comprising the nucleic acid sequence of TATCCACCTACAATCCTTGAAAGACCTTAAAC (SEQ ID NO: 27); and a reverse primer comprising the nucleic acid sequence of TTAGACATGTTTGAACTCCATTCTTCAGTGTA (SEQ ID NO: 6). In some embodiments, the forward primer comprises the nucleic acid sequence of GAAATTAATACGACTCACTATAGGTATCCACCTACAATCCTTGAAAGACCTTAAA C (SEQ ID NO: 5).

In some embodiments, a detection system further comprises an RNase inhibitor.

In some embodiments, a detection system further comprises an oligonucleotide comprising a detectable molecule that is detectable when the oligonucleotide is cleaved by the effector protein. In some embodiments, the detectable molecule is a quenched fluorophore that exhibits fluorescence when the oligonucleotide is cleaved by the effector protein. In some embodiments, the detectable molecule is a lateral flow reporter molecule comprising the nucleic acid sequence of 6FAM-mArArUrGrGrCmAmArArUrGrGrCmA-BIO (SEQ ID NO: 24).

In some embodiments, a detection system further comprises one or more reaction buffers.

In other aspects, the disclosure relates to diagnostic devices that comprise: (i) a detection system described herein; and (iii) one or more substrates. In some embodiments, the substrate is a flexible material substrate, a paper substrate, a flexible polymer-based substrate, or a lateral flow strip.

In some embodiments, a diagnostic device further comprises an imaging component.

In some embodiments, a diagnostic device further comprises an imaging analysis component. In some embodiments, the imaging analysis component comprises a lateral-flow quantification application.

In some embodiments, a diagnostic device comprises a compartment comprising a detection system and a polynucleotide positive control, wherein the polynucleotide positive control comprises a nucleic acid sequence that is bound or hybridized by a guide RNA of the detection system. In some embodiments, the polynucleotide positive control comprises the nucleic acid sequence of

(SEQ ID NO: 28)
ATGAAGAAAAGTGGTGTTCTTTTCCTCTTGGGCATCATCTTGCTGGTTCT
GATTGGAGTGCAAGGAACCCCAGTAGTGAGAAAGGGTCGCTGTTCCTGCA
TCAGCACCAACCAAGGGACTATCCACCTACAATCCTTGAAAGACCTTAAA
CAATTTGCCCCAAGCCCTTCCTGCGAGAAAATTGAAATCATTGCTACACT
GAAGAATGGAGTTCAAACATGTCTAAACCCAGATTCAGCAGATGTGAAGG
AACTGATTAAAAAGTGGGAGAAACAGGTCAGCCAAAAGAAAAAGCAAAAG
AATGGGAAAAAACATCAAAAAAAGAAAGTTCTGAAAGTTCGAAAATCTCA
ACGTTCTCGTCAAAAGAAGACTACATAA.

In some embodiments, the polynucleotide positive control comprises the nucleic acid sequence of

(SEQ ID NO: 23)
GAAATTAATACGACTCACTATAGGATGAAGAAAAGTGGTGTTCTTTT
CCTCTTGGGCATCATCTTGCTGGTTCTGATTGGAGTGCAAGGAACCC
CAGTAGTGAGAAAGGGTCGCTGTTCCTGCATCAGCACCAACCAAGGG
ACTATCCACCTACAATCCTTGAAAGACCTTAAACAATTTGCCCCAAG
CCCTTCCTGCGAGAAAATTGAAATCATTGCTACACTGAAGAATGGAG
TTCAAACATGTCTAAACCCAGATTCAGCAGATGTGAAGGAACTGATT
AAAAAGTGGGAGAAACAGGTCAGCCAAAAGAAAAAGCAAAAGAATGG
GAAAAAACATCAAAAAAAGAAAGTTCTGAAAGTTCGAAAATCTCAAC
GTTCTCGTCAAAAGAAGACTACATAA.

In some embodiments, a device comprises a compartment comprising a detection system and a polynucleotide negative control, wherein the polynucleotide negative control comprises a nucleic acid sequence that is not bound or hybridized by a guide RNA of the detection system.

In other aspects, the disclosure relates to kits that comprise a detection system described herein or a diagnostic device described herein.

In some embodiments, a kit further comprises a polynucleic acid isolation component. In some embodiments, the polynucleic acid isolation component comprises tris(2-carboxyethyl)phosphine, EDTA, or a combination thereof. In some embodiments, the polynucleic acid isolation component comprises a purification column.

In some embodiments, a kit comprises a polynucleotide positive control, wherein the polynucleotide positive control comprises a nucleic acid sequence that is bound or hybridized by a guide RNA of the detection system. In some embodiments, the polynucleotide positive control comprises the nucleic acid sequence of

(SEQ ID NO: 28)
ATGAAGAAAAGTGGTGTTCTTTTCCTCTTGGGCATCATCTTGCTGGT
TCTGATTGGAGTGCAAGGAACCCCAGTAGTGAGAAAGGGTCGCTGTT
CCTGCATCAGCACCAACCAAGGGACTATCCACCTACAATCCTTGAAA
GACCTTAAACAATTTGCCCCAAGCCCTTCCTGCGAGAAAATTGAAAT
CATTGCTACACTGAAGAATGGAGTTCAAACATGTCTAAACCCAGATT
CAGCAGATGTGAAGGAACTGATTAAAAAGTGGGAGAAACAGGTCAGC
CAAAAGAAAAAGCAAAAGAATGGGAAAAAACATCAAAAAAAGAAAGT
TCTGAAAGTTCGAAAATCTCAACGTTCTCGTCAAAAGAAGACTACAT
AA.

In some embodiments, the polynucleotide positive control comprises the nucleic acid sequence of

(SEQ ID NO: 23)
GAAATTAATACGACTCACTATAGGATGAAGAAAAGTGGTGTTCTTTT
CCTCTTGGGCATCATCTTGCTGGTTCTGATTGGAGTGCAAGGAACCC
CAGTAGTGAGAAAGGGTCGCTGTTCCTGCATCAGCACCAACCAAGGG
ACTATCCACCTACAATCCTTGAAAGACCTTAAACAATTTGCCCCAAG
CCCTTCCTGCGAGAAAATTGAAATCATTGCTACACTGAAGAATGGAG
TTCAAACATGTCTAAACCCAGATTCAGCAGATGTGAAGGAACTGATT
AAAAAGTGGGAGAAACAGGTCAGCCAAAAGAAAAAGCAAAAGAATGG
GAAAAAACATCAAAAAAAGAAAGTTCTGAAAGTTCGAAAATCTCAAC
GTTCTCGTCAAAAGAAGACTACATAA.

In some embodiments, a kit comprises a compartment comprising a detection system and a polynucleotide negative control, wherein the polynucleotide negative control comprises a nucleic acid sequence that is not bound or hybridized by a guide RNA of the detection system.

In other aspects, the disclosure relates to methods for detecting a target molecule in a sample. In some embodiments, the method comprises contacting the sample with a detection system described herein, a diagnostic device described herein, or a kit described herein.

In some embodiments, the sample is a urine sample, a blood sample, a serum sample, or a plasma sample from a patient having an organ transplant. In some embodiments, the organ transplant is a renal transplant.

In some embodiments, the method comprises purifying polynucleotides from the sample.

In some embodiments, the method comprises amplifying the target molecule using an isothermal recombinase polymerase amplification method (RPA).

In some embodiments, RNAses in the sample are inhibited.

In other aspects, the disclosure relates to methods of detecting an opportunistic post-transplantation viral infection. In some embodiments, the method comprises: contacting nucleic acids from a sample obtained from a transplant patient with a detection system described herein, a diagnostic device described herein, or a kit described herein; wherein the target molecule is a polynucleic acid that is indicative of an opportunistic post-translational viral infection.

In some embodiments, the target is a viral DNA molecule. In some embodiments, the target molecule is selected from the group consisting of BK polyomavirus DNA, cytomegalovirus DNA, or a combination thereof.

In some embodiments, the sample is a urine sample, a blood sample, a serum sample, or a plasma sample from a patient having an organ transplant. In some embodiments, the organ transplant is a renal transplant.

In some embodiments, the method comprises purifying polynucleotides from the sample.

In some embodiments, the method comprises amplifying the target molecule using an isothermal recombinase polymerase amplification method (RPA).

In some embodiments, RNAses in the sample are inhibited.

In other aspects, the disclosure relates to methods for identifying a subject having BK nephropathy. In some embodiments, the method comprises: contacting nucleic acids from a sample obtained from a patient with a detection system described herein, a device described herein, or a kit described herein; wherein the target molecule is a polynucleic acid that is indicative of BK nephropathy.

In some embodiments, the target is a viral DNA molecule. In some embodiments, the target is BK polyomavirus DNA.

In some embodiments, the method comprises purifying polynucleotides from the sample.

In some embodiments, the method comprises amplifying the target molecule using an isothermal recombinase polymerase amplification method (RPA).

In some embodiments, RNAses in the sample are inhibited.

In other aspects, the disclosure relates to methods for the monitoring of transplant rejection. In some embodiments, the method comprises: contacting nucleic acids from a sample obtained from a transplant patient with a detection system described herein, a diagnostic device described herein, or a kit described herein; wherein the target molecule is a polynucleic acid that is indicative of transplant rejection.

In some embodiments, the target molecule is a cytokine mRNA. In some embodiments, the target molecule is a CXCL9 mRNA, CXCL10 mRNA, and a combination thereof.

In some embodiments, the sample is a urine sample, a blood sample, a serum sample, or a plasma sample from a patient having an organ transplant. In some embodiments, the organ transplant is a renal transplant.

In some embodiments, the method comprises purifying polynucleotides from the sample.

In some embodiments, the method comprises amplifying the target molecule using an isothermal recombinase polymerase amplification method (RPA).

In some embodiments, RNAses in the sample are inhibited.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Progression of kidney transplant injury, detection by routine clinical labs (e.g., creatinine level) and effectiveness of intervention according to timing of diagnosis.

FIG. 2. Illustration of the point of care test for kidney rejection and BKV infection.

FIGS. 3A-3B. Detection of BKV DNA. FIG. 3A. The diagnostic DNA standard from ATCC was serially diluted as indicated in Copies/μl (C/μl) or attomoles [aM] and subjected to RPA and CRISPR-based detection. The output of the reaction was measured as relative fluorescent units (RFUs) over time. Grey indicates 6 C/μl as input, which corresponds to the lower reactivation cut-off of BK virus in clinical settings. FIG. 3B. Graphs of BKV DNA detection by CRISPR/Cas13 according to BKV concentration. Triplicates, experiments repeated three times.

FIGS. 4A-4B. Detection of BKV DNA using CRISPR/Cas13. FIG. 4A. BKV was detected in different sample types from a 40-year-old transplant recipient with BKV nephropathy. Urine from a healthy non-transplanted subject is used as a control. BKV was detected in urine pellet, urine supernatant, serum and plasma of the transplanted patient. FIG. 4B. Detection of BKV virus DNA in multiple samples from different patients (n=25). BKV virus copy numbers were measured in a CLIA certified lab with standard diagnostic PCR. The assay demonstrated 100% sensitivity in the virus detection.

FIGS. 5A-5C. RNase inactivation and mRNA detection in urine. FIG. 5A. Representative results of detection of CXCL9 in water and urine, demonstrating importance of applying preservation methods for the mRNA in urine. FIG. 5B. Urine samples were processed under various conditions for mRNA preservation, followed by CXCL9 detection. FIG. 5C. Dilution experiment with CXCL9 detection by CRISPR/Cas13 in triplicates (RFU).

FIG. 6. Representative lateral flow readout of BK virus infection. Urine of healthy (left stick) and BK virus infected (right stick) patients was tested for presence of BK virus DNA with CRISPR/Cas13 and a lateral flow readout. Arrows indicated the positive test band and the control band.

FIG. 7. Flowchart outlining study progression.

FIG. 8. List of challenges in biomarker research in transplantation.

FIGS. 9A-9J. Sherlock detection of viral DNA (BKV, CMV) and cytokine mRNA (CXCL9). FIG. 9A. Schematic illustrating Sherlock-based detection of cytomegalovirus (CMV), BK virus (BKV) and CXCL9. RPA amplified targets are recognized by Cas13, whose collateral cleavage of reporter oligos releases quenched fluorescence measured on a plate reader. (rt)RPA: (reverse transcription) recombinase polymerase amplification, T7: T7 promoter. FIG. 9B. Conservation of the BKV target region among all BKV strains. STA: small T antigen. FWD: RPA forward primer, REV: RPA reverse primer, SP: crRNA spacer. FIG. 9C. Sherlock detection of the ATCC quantitative BKV synthetic standard (Dunlop strain) at the indicated concentrations. Each symbol represents the mean of an independent experiment. Asterisks indicate significant difference to no template control (CTL) as assessed by students t-test. Error bars: SEM. FIG. 9D. Testing for BKV virus in urine and plasma patients' samples processed with HUDSON. All samples were analyzed for the virus copy number by gold standard PCR in a CLIA certified diagnostics lab (y-axis). FIG. 9E. Conservation of the CMV target region in the UL54 gene among all CMV strains. FWD: RPA forward primer, REV: RPA reverse primer, SP: crRNA spacer. FIG. 9F. Sherlock detection of the ATCC quantitative CMV standard (strain AD-169). Each symbol represents the mean of an independent experiment. Asterisks indicate significant difference to no template control (CTL) as assessed by students t-test. Error bars: SEM. FIG. 9G. Testing for CMV virus in plasma patients' samples isolated with a commercial column-based kit or with HUDSON. All samples were analyzed for the virus copy number by gold standard PCR in a CLIA certified diagnostics lab (y-axis). FIG. 9H. Detection of synthetic CXCL9 mRNA with Cas13 or rtRPA-Cas13. Each symbol represents the average of 3 biological replicates. Error bars: SD. FIGS. 9I. Quantification of CXCL9 mRNA in patients' urine relative to a synthetic CXCL9 standard. Rejection: biopsy proven kidney rejection. No rejection: Stable graft function. Violin plots showing data distribution with lines indicating the median and quartiles. FIG. 9J. Quantification of CXCL9 mRNA in patients' urine. Each symbol represents a different, independent patient sample. p<0.05 (*), p<0.01 (**), p<0.001 (***), p<0.0001 (****) (FIGS. 9C, 9F, 9H-J).

FIGS. 10A-10G. Point of care read-out and monitoring of transplant patients. FIG. 10A. Schematic illustrating the POCT workflow. FIGS. 10B-10C. Detection of CMV (FIG. 10B) and BKV (FIG. 10C) in patient plasma samples with lateral flow strips. Viral copy number (C/ml) as measured by PCR. FIG. 10D. Schematic (left) and performance (right) of the RPA-CRISPR/Cas13 one pot reaction detecting the ATCC quantitative BKV synthetic standard (Dunlop strain) at the indicated concentrations. Each symbol represents an independent one pot reaction. Asterisks indicate significant difference to no template control (CTL) as assessed by students t-test. Error bars: SD. p<0.05 (*), p<0.01 (**), p<0.001 (***), p<0.0001 (****). FIG. 10E. POCT-like infection monitoring of BKV infection over the indicated time-period in a transplant patient with BKV nephropathy. BKV viral load in Copies/ml as quantified by PCR (left y axis): (−) Sherlock negative POCT, (+) Sherlock positive POCT. FIGS. 10F-10G. POCT-like rejection monitoring of 2 kidney transplant patients undergoing organ rejection. CXCL9 expression relative to standard 18S rRNA as measured by qRT-PCR (left y axis). Arrows indicate time-points of kidney biopsies and histo-pathological diagnosis; serum creatinine in mg/dl (right y axis) (FIGS. 10E-10G).

FIGS. 11A-11B. Assay design and primer optimization. FIG. 11A. Workflow of target identification, RPA primer selection and crRNA testing. FIG. 11B. RPA primer dilution matrix. The Sherlock fluorescence signal of BKV detection at the indicated target concentrations is depicted as color intensity relative to the highest signal (100). Forward and reverse primer concentrations for RPA as indicated.

FIGS. 12A-12C. Sample processing and comparison of different specimens. FIG. 12A. Detection of BKV in the indicated specimen from the same patient compared to urine from a healthy control. N.s. non-significant, as assessed by Student's t-test. Error bars: SD. FIG. 12B. Comparison of different sample processing methods. HUDSON: 95° C., 10 mins in presence of 100 mM TCEP and 1 mM EDTA. TweenBO: Room temperature in presence of 1% (v/v) Tween 80. Reischl et al.: 95° C., 10 mins in presence of TritonX100 1% (v/v), Tween20 0.5% (v/v), TE (10 mM/1 mM). Fluorescence was normalized to the highest signal (HUDSON, 100). Asterisks indicate significant differences as assessed by one-way ANOVA and Tukey's multiple comparisons test. p<0.05 (*), p<0.01 (**), p<0.001 (***), p<0.0001 (****). Error bars: SD. FIG. 12C. Comparison of different HUDSON based protocols in presence of 100 mM TCEP and 1 mM EDTA. Dashed line indicates curve fit and 95% confidence band based on non-linear regression.

FIGS. 13A-13C. Optimization of the one-pot reaction. FIG. 13A. Effect of CRISPR and T7 transcription reagents upon addition to the RPA reaction. CRISPR reagents were added pooled (+CRISPR reagents) or as a pool with one component missing (+CRISPR reagents-1). Asterisks indicate significant differences to the RPA control without any additions (RPA CTL) as assessed by one-way ANOVA and Dunnett's multiple comparisons test. p<0.05 (*), p<0.01 (**), p<0.001 (***), p<0.0001 (****). N.s. non-significant. FIG. 13B. Concentration matrix of rNTPs and dNTPs in the one-pot reaction. The fluorescence signal is depicted as color intensity relative to the highest signal. FIG. 13C. Testing of the indicated MgOAc concentrations in the one-pot reaction using 1.8 mM dNTPs and 0.5 mM rNTPs. Different letters indicate significant differences between groups as assessed by one-way ANOVA and Tukey's multiple comparisons test.

FIGS. 14A-14G. CRISPR diagnostics enable single-molecule detection of BKV and CMV DNA. FIG. 14A. Schematic illustrating the assay. T7P, T7 promoter; F, RPA forward primer; R, RPA reverse primer. FIG. 14B. BKV target-region conservation among all BKV strains. SP, crRNA spacer. FIG. 14C. CRISPR-based detection of the BKV synthetic standard at indicated concentrations. Data are mean±s.e.m., n>4 independent experiments. RFU, relative fluorescence units. FIG. 14D. CRISPR-based detection of the BKV synthetic standard in the attomolar range. Data are mean±s.d. of three independent reactions. FIG. 14E. CMV target region conservation among all CMV strains. FIG. 14F. CRISPR-based detection of the CMV standard. Data are mean±s.e.m., n>3 independent experiments. FIG. 14G. CRISPR-based detection of the CMV synthetic standard in the attomolar range. Data are mean±s.d. of three independent reactions. Difference from control assessed by two-tailed Student's t-test; NS, not significant; *P<0.05, **P<0.01, ***P<0.001 and ****P<0.0001 (FIGS. 14C and 14F).

FIGS. 15A-15D. CRISPR detects BKV and CMV from human urine and blood samples over a wide range of viral loads. FIG. 15A. Testing for BKV virus in human urine (n=31) and plasma (n=36) samples, processed with HUDSON. Virus copy numbers were assessed by qPCR and expressed as DNA copies per ml. FIG. 15B. Confusion matrix of samples tested in FIG. 15A. FIG. 15C. Testing for CMV virus from plasma samples as in a (n=40), isolated either with HUDSON or a commercial column-based kit. Virus copy-numbers were assessed by qPCR. FIG. 15D. Confusion matrix of samples tested in FIG. 15C.

FIGS. 16A-16E. CRISPR-based diagnostics can detect CXCL9 mRNA as an indicator for acute cellular rejection. FIG. 16A. Schematic illustrating the assay. FIG. 16B. Detection of CXCL9 mRNA with Cas13 only or Cas13 with RT-RPA-CRISPR. Data are mean±s.d. of three independent reactions. The experiment was repeated three times. FIG. 16C. Top: CRISPR-based detection of CXCL9 mRNA in urine sample. The dashed line indicates the cut-off separating CRISPR-positive samples from CRISPR-negative samples. Rejection status was determined by kidney biopsy. In box plots, the center line is the median, boxes encompass second and third quartiles, whiskers represent the range of the data and crosses show the mean. Each symbol represents an independent patient sample, n=14 rejection samples, n=17 no-rejection samples. Bottom: confusion matrix for detection of rejection by CRISPR-based CXCL9 test. FIG. 16D. Frequency distribution of signal intensities for the no-rejection and rejection groups. FIG. 16E. Area under the receiver-operating-characteristic curve (AUC), assessing the accuracy of CRISPR-based rejection diagnostics (AUC of 1 indicates perfect discriminatory value; 0.5 or less indicates no discriminatory value). Difference from control assessed by two-tailed Student's t-test. NS, not significant; *P<0.05, **P<0.01, ***P<0.001 and ****P<0.0001 (FIGS. 16B-16C).

FIGS. 17A-17H. Lateral flow enables detection of viral DNA (BKV, CMV) for point-of-care testing. FIG. 17A. Schematic illustrating the assay. FIGS. 17B-17C. Lateral-flow-based detection of CMV (FIG. 17B) and BKV (FIG. 17C) synthetic standards at the indicated concentrations. Data are mean±s.d. of three independent reactions. Dashed line, band-intensity cut-off discriminating a positive assay result from a negative. FIGS. 17D-17E. Detection of CMV (FIG. 17D) and BKV (FIG. 17E) in patient plasma samples with lateral-flow strips. Viral copy number was measured by qPCR. The boxed areas below indicate whether smartphone-based band detection was positive (tick) or negative (cross). FIG. 17F. Top: monitoring of BKV infection (BKV viral load quantified by qPCR) over the indicated time period in a transplant recipient with BKV nephropathy. Bottom: Lateral-flow-based detection of BKV in urine at the indicated time points. Neg, negative control; pos, positive control. FIG. 17G. Signal variability of the lateral-flow detection using the same ten BKV-negative or BKV-positive urine samples as in f on days 1-3. Data are mean±s.d., n=10 samples. Letters indicate groups that are significantly different from each other; one-way ANOVA and Tukey's multiple comparisons test. FIG. 17H. Histograms indicating the frequency distribution of lateral-flow signals shown in FIG. 17G.

FIGS. 18A-18C. Monitoring of CXCL9 mRNA levels with lateral flow. FIG. 18A. Lateral-flow-based detection of CXCL9 synthetic RNA at the indicated concentrations. Dashed line, band-intensity cut-off discriminating a positive from a negative assay result. Data are mean±s.d. of three independent reactions. FIGS. 18B-18C. Monitoring of two kidney-transplant recipients undergoing organ rejection and treatment. Top: squares and left axis, CXCL9 expression measured by quantitative PCR with reverse transcription (RT-qPCR); circles and right axis, serum creatinine; ACR, acute cellular rejection; CCR, chronic cellular rejection. Arrows indicate time points of kidney biopsies. Bottom: lateral-flow assays for CXCL9 mRNA in urine.

FIGS. 19A-19B. Target genes and primer optimization. FIG. 19A. Target genes tested for detection of BKV or CMV infection and rejection. FIG. 19B. RPA primer dilution matrix. The Sherlock fluorescence signal of BKV detection (ATCC synthetic standard) at the indicated target concentrations is depicted as color intensity relative to the highest signal (100). Forward and reverse primer concentrations for RPA as indicated.

FIGS. 20A-20C. Comparison of different specimen types and sample processing. FIG. 20A. Detection of BKV in the indicated specimens from the same patient compared to urine from a healthy control. Different small letters indicate significant differences as assessed by one-way ANOVA and Tukey's multiple comparisons test. Symbols: mean±SD of 3 independent reactions. FIG. 20B. Comparison of sample processing by the HUDSON method and incubation with Tween80 1% for 20 min at room temperature. Tested on 3 different CMV negative patient samples to test for unspecific background noise and one CMV positive patient sample. Fluorescence was normalized to the highest signal (HUDSON, 100). Asterisks indicate significant differences as assessed by Student's two-tailed t-test. Symbols: mean±SD of 3 independent reactions. FIG. 20C. Comparison of different HUDSON-based protocols on a CMV negative and a CMV positive patient sample. Asterisks indicate significant differences to the 95° C./10 min (3884 IU/mL) condition as assessed by one-way ANOVA and Dunnett's multiple comparisons test. Symbols: mean±SD of 3 independent reactions. n.s. not significant, p<0.05 (*), p<0.01 (**), p<0.001 (***), p<0.0001 (****) (FIGS. 20B-20C).

FIGS. 21A-21F. CXCL9 mRNA and protein levels in rejection patients and controls. FIG. 21A. qPCR-based detection of CXCL9 mRNA in rejection patients (n=14) and no rejection control patients (n=17). qPCR(−) indicates qPCR negative tests, and qPCR(+) indicates qPCR positive test results. The dashed line indicates the cut-off differentiating between a negative and positive test result. FIG. 21B. Sensitivity and specificity of rejection detection by qPCR calculated using the cut-off value depicted in FIG. 21A. FIG. 21C. Area under the receiver-operating-characteristic (ROC) curve (AUC) assessing the accuracy of qPCR-based rejection diagnostics (1 indicates perfect discriminatory value; 0.5 or less indicates no discriminatory value). FIG. 21D. ELISA-based detection of CXCL9 protein in rejection patients (n=14) and no rejection control patients (n=11). The tested samples were the same as depicted in FIG. 21A (rejection) or a subset of them (no rejection). ELISA(−) indicates ELISA negative tests, and ELISA(+) indicates ELISA positive test results. FIG. 21E. Confusion matrix indicating the sensitivity and specificity of ELISA-based rejection detection. FIG. 21F. Area under the receiver-operating-characteristic (ROC) curve (AUC) assessing the accuracy of ELISA-based rejection diagnostics (1 indicates perfect discriminatory value; 0.5 or less indicates no discriminatory value). Box plot lines: median and quartiles, whiskers: data range, crosses: averages. Each symbol represents a different, independent patient sample. Asterisks: significant difference to control as assessed by Student's two-tailed t-test. p<0.01 (**) (FIGS. 21A and 21D).

FIGS. 22A-22D. Influence of temperature and incubation time on lateral-flow band intensity. FIG. 22A. Incubation of the CRISPR reaction detecting the CMV synthetic standard at the indicated concentrations for the indicated reaction times. The lateral-flow-based readout was quantified as the ratio of test/control band. The dashed line indicates the assay's cut-off. Symbols: mean±SD of 3 independent reactions. Asterisks indicate significant differences to the 60 min control reaction time as assessed by Student's two-tailed t-test. FIG. 22B. Images of the lateral-flow assays quantified in FIG. 22A. FIG. 22C. Incubation at different temperatures for the detection of the CMV synthetic standard at the indicated concentrations with lateral flow. Symbols: mean±SD of 3 independent reactions. Asterisks indicate significant differences to the 21° C. control reaction as assessed by Student's two-tailed t-test. FIG. 22D. Images of the lateral-flow assays quantified in FIG. 22D. n.s. not significant, p<0.05 (*), p<0.01 (**), p<0.001 (***), p<0.0001 (****).

FIGS. 23A-23E. Optimization of the one-pot reaction. FIG. 23A. Schematic illustration of the one-pot assay. FIG. 23B. Effect of CRISPR and T7 transcription reagents upon addition to the RPA reaction. CRISPR reagents were added pooled (+CRISPR reagents) or as a pool with one component missing (+CRISPR reagents-1). CMV synthetic DNA served as target (500 aM). A separate CRISPR/T7 reaction served as readout. Fluorescence was normalized to the highest signal (RPA CTL, 100). Asterisks indicate significant differences to the RPA control without any additions (RPA CTL) as assessed by one-way ANOVA and Dunnett's multiple comparisons test. Symbols: mean±SD of 3 independent reactions. FIG. 23C. Concentration matrix of rNTPs and dNTPs in the one-pot reaction. The fluorescence signal is depicted as color intensity relative to the highest signal. Asterisks indicate significant differences as assessed by Student's two-tailed t-test. FIG. 23D. Testing of the indicated MgOAc concentrations in the one-pot reaction. CMV synthetic DNA served as target (500 aM). Different letters indicate significant differences between groups as assessed by one-way ANOVA and Tukey's multiple comparisons test. Symbols: mean±SD of 3 independent reactions. FIG. 23E. One-pot reaction detecting the ATCC quantitative BKV synthetic standard (Dunlop strain) at the indicated concentrations. Symbols: mean±SD of 3 independent reactions. Asterisks indicate significant differences to no template control (CTL) as assessed by Student's two-tailed t-test. p<0.05 (*), p<0.01 (**), p<0.001 (***), p<0.0001 (****).

### DETAILED DESCRIPTION

Since the first successful kidney transplantation in 1954, substantial improvements in short-term outcomes have been achieved in organ transplantation. However, there has been less progress in long-term outcomes, with more than half of the transplanted organs being lost after 10 yr (3, 60). Opportunistic infections and transplant-organ rejection are leading causes of graft loss, requiring careful adjustment of immunosuppression and life-long monitoring of post-transplant patients (61).

Current diagnostics, however, involve the use of expensive laboratory equipment and intricate multistep protocols, leading to limited availability, high costs and slow turnaround time (62, 63). Diagnosis of infections using PCR can take several days in clinical settings, and rejection diagnostics require invasive biopsies and histopathological analysis. These factors result in delays in pertinent diagnoses and increase the risk of irreversible allograft injury, especially in resource-limited settings. POC or at-home testing could significantly reduce associated costs and enable more frequent monitoring, which would lead to earlier diagnosis and treatment of graft dysfunction and common infections.

Described herein are CRISPR-based diagnostic tools that were developed for cytomegalovirus (CMV) and BK polyomavirus (BKV) infection, two common opportunistic viruses that are highly relevant for renal-transplant patients (64) and other immunocompromised patients (65, 66). Testing of more than 100 clinical specimens from patients infected with BKV and CMV over a wide range of virus loads revealed high diagnostic accuracy. These techniques involve the SHERLOCK methodology, which has been described previously. See e.g., U.S. Pat. Nos. 10,266,886 and 10,266,887, and US Published Application Nos. 2018/0274017, 2018/0298445; 2019/0144929; 2020/0181720, the entireties of each of which are incorporated herein by reference. The capability of SHERLOCK was extended to the detection of human CXCL9 mRNA, a biomarker indicative of rejection in renal-transplant recipients (6, 7, 10). These CRISPR-Cas13-based technologies are broadly applicable for personalized medicine diagnostics, where repeated testing of biomarkers indicative of disease activity is key to early and effective secondary prevention.

### I. Nucleic Acid Detection Systems.

In some aspects, the disclosure relates to nucleic acid detection systems comprising a CRISPR component comprising: (i) an effector protein and/or a polynucleotide encoding an effector protein; and (ii) a guide RNA, and/or a polynucleotide encoding a guide RNA, that binds or hybridizes to a corresponding target molecule.

A. Effector Proteins

As described herein, the term “effector protein” refers to a nuclease that has promiscuous activity (i.e., collateral activity) after it cleaves a nucleic acid in a guide-RNA-mediated fashion (i.e., programmable RNase activity). In some embodiments, the effector protein is a Cas13 enzyme.

Cas13 enzymes function using an approximately 64-nt guide RNA to encode target specificity. A Cas13 protein complexes with the guide RNA via recognition of a short hairpin in the crRNA, and target specificity is encoded by a 28-30-nt spacer that is complementary to a target molecule. In addition to programmable RNase activity, Cas13 enzymes exhibit collateral activity after recognition and cleavage of a target molecule, leading to non-specific degradation of any nearby transcripts regardless of complementarity to the spacer.

Exemplary Cas13 enzymes are known in the art and include Cas13a (formerly C2c2), Cas13b, Cas13c, and Cas13d. In some embodiments, a CRISPR component comprises a Cas13a protein, a Cas13b protein, a Cas13c protein, or a Cas13d protein. In some embodiments, a CRISPR component comprises two or more effector proteins (i.e., two or more distinct effector proteins) and/or a polynucleotide encoding two or more effector proteins. For example, in some embodiments, a CRISPR component comprises two or more of a Cas13a protein, a Cas13b protein, a Cas13c protein, or Cas13d protein.

In some embodiments, a CRISPR component comprises a Cas13a protein. In some embodiments, the Cas13b protein is Cas13a from Leptotrichia wadei (i.e., LwaCas13a).

Exemplary LwaCas13a Amino Acid Sequence
(SEQ ID NO: 29):
MYMKITKIDGVSHYKKQDKGILKKKWKDLDERKQREKIEARYNKQIE
SKIYKEFFRLKNKKRIEKEEDQNIKSLYFFIKELYLNEKNEEWELKN
INLEILDDKERVIKGYKFKEDVYFFKEGYKEYYLRILFNNLIEKVQN
ENREKVRKNKEFLDLKEIFKKYKNRKIDLLLKSINNNKINLEYKKEN
VNEEIYGINPTNDREMTFYELLKEIIEKKDEQKSILEEKLDNFDITN
FLENIEKIFNEETEINIIKGKVLNELREYIKEKEENNSDNKLKQIYN
LELKKYIENNFSYKKQKSKSKNGKNDYLYLNFLKKIMFIEEVDEKKE
INKEKFKNKINSNFKNLFVQHILDYGKLLYYKENDEYIKNTGQLETK
DLEYIKTKETLIRKMAVLVSFAANSYYNLFGRVSGDILGTEVVKSSK
TNVIKVGSHIFKEKMLNYFFDFEIFDANKIVEILESISYSIYNVRNG
VGHFNKLILGKYKKKDINTNKRIEEDLNNNEEIKGYFIKKRGEIERK
VKEKFLSNNLQYYYSKEKIENYFEVYEFEILKRKIPFAPNFKRIIKK
GEDLFNNKNNKKYEYFKNFDKNSAEEKKEFLKTRNFLLKELYYNNFY
KEFLSKKEEFEKIVLEVKEEKKSRGNINNKKSGVSFQSIDDYDTKIN
ISDYIASIHKKEMERVEKYNEEKQKDTAKYIRDFVEEIFLTGFINYL
EKDKRLHFLKEEFSILCNNNNNVVDFNININEEKIKEFLKENDSKTL
NLYLFFNMIDSKRISEFRNELVKYKQFTKKRLDEEKEFLGIKIELYE
TLIEFVILTREKLDTKKSEEIDAWLVDKLYVKDSNEYKEYEEILKLF
VDEKILSSKEAPYYATDNKTPILLSNFEKTRKYGTQSFLSEIQSNYK
YSKVEKENIEDYNKKEEIEQKKKSNIEKLQDLKVELHKKWEQNKITE
KEIEKYNNTTRKINEYNYLKNKEELQNVYLLHEMLSDLLARNVAFFN
KWERDFKFIVIAIKQFLRENDKEKVNEFLNPPDNSKGKKVYFSVSKY
KNTVENIDGIHKNFMNLIFLNNKFMNRKIDKMNCAIWVYFRNYIAHF
LHLHTKNEKISLISQMNLLIKLFSYDKKVQNHILKSTKTLLEKYNIQ
INFEISNDKNEVFKYKIKNRLYSKKGKMLGKNNKFEILENEFLENVK
AMLEYSE

In some embodiments, a CRISPR component comprises a LwaCas13a protein or a functional variant thereof (and/or a polynucleotide encoding a LwaCas13a protein or a functional variant thereof). For example, in some embodiments, a CRISPR component comprises an effector protein (and/or a polynucleotide encoding an effector protein) having programmable RNase activity and promiscuous activity and having at least 60%, at least 65%, at least 70%, at least 75%, at least 80%, at least 85%, at least 90%, at least 91%, at least 92%, at least 93%, at least 94%, at least 95% at least 96%, at least 97%, at least 98%, at least 99%, at least 99.5%, or 100% identity with SEQ ID NO: 29.

Methods of determining the extent of identity between two sequences (e.g., two amino acid sequences or two polynucleic acids) are known to those having ordinary skill in the art. One exemplary method is the use of Basic Local Alignment Search Tool (BLAST®) software with default parameters (blast.ncbi.nlm.nih.gov/Blast.cgi).

B. Guide RNA

As described herein, the term “guide RNA” refers to an oligonucleotide that binds or hybridizes to a target nucleic acid and is recognized/bound by an effector protein (e.g., Cas13). When complexed with an effector protein and bound or hybridized to a target nucleic acid, the target nucleic acid is cleaved in a guide-RNA-mediated fashion (i.e., programmable RNase activity).

In some embodiments, a CRISPR component comprises a guide RNA directed to a single target. In other embodiments, a CRISPR component comprises two or more guide RNAs directed to different targets. For example, in some embodiments, a CRISPR component comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different guide RNAs (i.e., having unique nucleic acid sequences). In some embodiments, a CRISPR component comprises 1-5, 1-10, 1-15, 1-20, 2-5, 2-10, 2-15, 2-20, 3-5, 3-10, 3-15, 2-20, 4-5, 4-10, 2-15, 4-20, 5-10, 5-15, 5-20, 6-10, 6-15, 6-20, 7-10, 7-15, 7-20, 8-10, 8-15, 8-20, 9-10, 9-15, or 9-20 different guide RNAs (i.e., having unique nucleic acid sequences). In some embodiments, a CRISPR component comprises 2, 3, 4, 5, 6, 7, 8, 9, 10, 11, 12, 13, 14, 15, 16, 17, 18, 19, or 20 different guide RNAs (i.e., having unique nucleic acid sequences).

Guide RNAs comprise a spacer sequence that is complementary to the target region of a target molecule. The spacer sequence of a guide RNA is typically 28-30-nt in length. In some embodiments, a spacer sequence is at least 25-nt in length, at least 26-nt in length, at least 27-nt in length, at least 28-nt in length, at least 29-nt in length, at least 30-nt in length, at least 31-nt in length, at least 32-nt in length, or at least 33-nt in length. In some embodiments, a spacer sequence is 25-33, 25-32, 25-31, 25-30, 26-33, 26-32, 26-31, 26-30, 27-33, 27-32, 27-31, 27-30, 28-33, 28-32, 28-31, or 28-30-nt in length. In some embodiments, a spacer sequence is 26, 26, 27, 28, 29, 30, 31, 32, or 33-nt in length.

In some embodiments, a CRISPR component comprises: a guide RNA comprising the nucleic acid sequence of TTGCTACTGCATTGACTGCTTCACACAG (SEQ ID NO: 20) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 20; a guide RNA comprising the nucleic acid sequence of ACGAGGTCCGTGTGGATCCGCTGACGCG (SEQ ID NO: 21) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 21; a guide RNA comprising the nucleic acid sequence of ATGATTTCAATTTTCTCGCAGGAAGGGC (SEQ ID NO: 22) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 22; or a combination thereof.

Guide RNAs may have varying lengths. Typically, a guide RNA is approximately 64-nt in length. In some embodiments, a guide RNA is at least 58-nt in length, at least 59-nt in length, at least 60-nt in length, at least 61-nt in length, at least 62-nt in length, at least 63-nt in length, at least 64-nt in length, at least 65-nt in length, at least 66-nt in length, at least 67-nt in length, or at least 68-nt in length. In some embodiments, a guide RNA is 60-68, 60-67, 60-65, 60-64, 61-68, 61-67, 61-66, 61-65, 61-64, 62-68, 62-67, 62-66, 62-65, 62-64, 63-68, 63-67, 63-66, 63-65, 63-64, 64-68, 64-67, or 64-65-nt in length. In some embodiments, a spacer sequence is 60, 61, 62, 63, 64, 65, 66, 67, or 68-nt in length.

In some embodiments, a CRISPR component comprises: a guide RNA comprising the nucleic acid sequence of GATTTAGACTACCCCAAAAACGAAGGGGACTAAAACTTGCTACTGCATTGACTG CTTCACACAG (SEQ ID NO: 17) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 17; a guide RNA comprising the nucleic acid sequence of GATTTAGACTACCCCAAAAACGAAGGGGACTAAAACACGAGGTCCGTGTGGATC CGCTGACGCG (SEQ ID NO: 18) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 18; a guide RNA comprising the nucleic acid sequence of GATTTAGACTACCCCAAAAACGAAGGGGACTAAAACATGATTTCAATTTTCTCGC AGGAAGGGC (SEQ ID NO: 19) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 19; or a combination thereof.

In some embodiments, a CRISPR component comprises a polynucleotide encoding a guide RNA and comprising the nucleic acid sequence of CTGTGTGAAGCAGTCAATGCAGTAGCAAGTTTTAGTCCCCTTCGTTTTTGGGGTA GTCTAAATCCCTATAGTGAGTCGTATTAATTTC (SEQ ID NO: 14) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 14; a polynucleotide encoding a guide RNA and comprising the nucleic acid sequence of CGCGTCAGCGGATCCACACGGACCTCGTGTTTTAGTCCCCTTCGTTTTTGGGGTA GTCTAAATCCCTATAGTGAGTCGTATTAATTTC (SEQ ID NO: 15) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 15; a polynucleotide encoding a guide RNA and comprising the nucleic acid sequence of GCCCTTCCTGCGAGAAAATTGAAATCATGTTTTAGTCCCCTTCGTTTTTGGGGTAG TCTAAATCCCTATAGTGAGTCGTATTAATTTC (SEQ ID NO: 16) or a nucleic acid sequence having at least 80%, at least 85%, at least 90%, at least 91%, at least 92%, at least 93%, at least 94%, at least 95%, at least 96%, at least 97%, at least 98%, at least 99% identity with SEQ ID NO: 16; or a combination thereof.

C. Target Molecule

As described herein, the term “target molecule” refers to a polynucleic acid molecule that is bound or hybridized by a guide RNA of the detection system and that is cleaved by an effector protein of the detection system in a guide-RNA-mediated fashion.

In some embodiments, a detection system is directed at a single target. In other embodiments, a detection system is directed at multiple different targets. For example, in some embodiments, a detection system is directed at at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different targets. In some embodiments, a detection system is directed at 1-5, 1-10, 1-15, 1-20, 2-5, 2-10, 2-15, 2-20, 3-5, 3-10, 3-15, 2-20, 4-5, 4-10, 2-15, 4-20, 5-10, 5-15, 5-20, 6-10, 6-15, 6-20, 7-10, 7-15, 7-20, 8-10, 8-15, 8-20, 9-10, 9-15, or 9-20 different targets. In some embodiments, a detection system is directed at 2, 3, 4, 5, 6, 7, 8, 9, 10, 11, 12, 13, 14, 15, 16, 17, 18, 19, or 20 different targets.

In some embodiments, a target molecule is indicative of rejection and/or infection of an organ transplant.

In some embodiments, a target molecule is a viral DNA. In some embodiments, the viral DNA is BK polyomavirus DNA. In some embodiments, the viral DNA is cytomegalovirus DNA.

In some embodiments, a target molecule is a cytokine mRNA. Examples or cytokine mRNAs are known to those having ordinary skill in the art. In some embodiments, a target molecule is a CXCL9 mRNA. In some embodiments, a target molecule is a CXCL10 mRNA.

D. Amplification Component

A nucleic acid detection system may comprise an amplification component that serves to amplify a target molecule prior to detection. An amplification component may comprise a polymerase and/or a primer.

(i) Polymerase

In some embodiments, an amplification component comprises a polymerase that facilitates amplification of a target molecule. In some embodiments an amplification component comprises multiple polymerases. For example, in some embodiments an amplification component comprises at least 2, at least 3, at least 4, or at least 5 different polymerases (i.e., polymerases having unique amino acid sequences).

In some embodiments, a polymerase is a DNA polymerase. Examples of DNA polymerases are known to those having ordinary skill in the art.

In some embodiments, a polymerase is an RNA polymerase. Examples of RNA polymerases are known to those having ordinary skill in the art. In some embodiments, the RNA polymerase is T7 RNA polymerase.

In some embodiments, amplification is performed using an isothermal recombinase polymerase amplification method (RPA), which uses a recombinase, a single-stranded DNA-binding protein and a strand-displacing polymerase. When amplifying RNA, the RPA method can also include a reverse transcriptase to transcribe RNA to DNA for isothermal amplification (RT-RPA). Subsequent to amplification of nucleic acids in a sample by RPA or RT-RPA, T7 RNA polymerase (or another RNA polymerase) can be used to produce RNA for recognition and cleavage by Cas13 effector protein(s) and guide RNA(s).

(ii) Primer

In some embodiments, an amplification component comprises a primer that facilitates amplification of a target molecule. In some embodiments, an amplification component comprises multiple primers. For example, in some embodiments, an amplification component comprises a primer pair (i.e., a forward and a reverse primer). In some embodiments, an amplification component comprises multiple unique primer pairs, e.g., 2, 3, 4, 5, 6, 7, 8, 9, or 10 unique primer pairs. In some embodiments, an amplification component comprises at least 2, at least 3, at least 4, at least 5, at least 6 at least 7, at least 8, at least 9, at least 10, at least 11, at least 12, at least 13, at least 14, at least 15, at least 16, at least 17, at least 18, at least 19, or at least 20 unique primers. In some embodiments, an amplification component comprises 2-4, 2-6, 2-8, 2-10, 4-6, 4-8, 4-10, 6-8, 6-10, or 8-10 unique primers. In some embodiments, an amplification component comprises 2, 3, 4, 5, 6, 7, 8, 9, 10, 11, 12, 13, 14, 15, 16, 17, 18, 19, or 20 unique primers.

When a detection system comprises a primer pair, the forward and reverse primer of the primer pair may be present in different concentrations. In some embodiments, the concentration of the reverse primer is greater than the concentration of the forward primer. For example, in some embodiments, the concentration of the reverse primer is 2 times, 3 times, 4 times, 5 times, 6 times, 7 times, or 8 times higher than the concentration of the forward primer. In some embodiments, the forward and reverse primer concentrations are 120 nM and 480 nM, respectively.

In some embodiments, the reverse primer of a primer pair may comprise an RNA polymerase promoter sequence. Exemplary RNA polymerase promoter sequences are known to those having ordinary skill in the art. In some embodiments, the RNA polymerase promoter sequence is a T7 polymerase promoter sequence. In some embodiments, the T7 polymerase promoter sequence comprises the nucleic acid sequence of GAAATTAATACGACTCACTATAGG (SEQ ID NO: 13) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 13.

In some embodiments, an amplification system comprises: a forward primer comprising the nucleic acid sequence of CATTGCAGAGTTTCTTCAGTTAGGTCTAAGCC (SEQ ID NO: 25) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 25; and a reverse primer comprising the nucleic acid sequence of AATTTTTAAGAAAAGAGCCCTTGGTTTGGATA (SEQ ID NO: 2) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 2. In some embodiments, the forward primer comprises the nucleic acid sequence of GAAATTAATACGACTCACTATAGGCATTGCAGAGTTTCTTCAGTTAGGTCTAAGC C (SEQ ID NO: 1) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 1.

In some embodiments, an amplification system comprises: a forward primer comprising the nucleic acid sequence of GCACCAGCCGAACGTGGTGATCCGCCGATCGATGAC (SEQ ID NO: 26) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 26; and a reverse primer comprising the nucleic acid sequence of CTATCAGCAACTGGACCATGGCCAGAAAAATCG (SEQ ID NO: 4) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 4. In some embodiments, the forward primer comprises the nucleic acid sequence of GAAATTAATACGACTCACTATAGGGCACCAGCCGAACGTGGTGATCCGCCGATC GATGAC (SEQ ID NO: 3) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 1.

In some embodiments, an amplification system comprises: a forward primer comprising the nucleic acid sequence of TATCCACCTACAATCCTTGAAAGACCTTAAAC (SEQ ID NO: 27) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 27; and a reverse primer comprising the nucleic acid sequence of TTAGACATGTTTGAACTCCATTCTTCAGTGTA (SEQ ID NO: 6) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 6. In some embodiments, the forward primer comprises the nucleic acid sequence of GAAATTAATACGACTCACTATAGGTATCCACCTACAATCCTTGAAAGACCTTAAA C (SEQ ID NO: 5) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 5.

E. Detectable Molecule

A nucleic acid detection system may comprise a detectable molecule that helps amplify detectable output such that one can more readily distinguish between the presence and absence of a target molecule. In some embodiments, a detection system comprises multiple distinct detectable molecules (i.e., detection molecules having a unique chemical compositions). For example, in some embodiments, a detection system comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 distinct detectable molecules. In some embodiments, a detection system comprises 1-5, 1-10, 1-15, 1-20, 2-5, 2-10, 2-15, 2-20, 3-5, 3-10, 3-15, 2-20, 4-5, 4-10, 2-15, 4-20, 5-10, 5-15, 5-20, 6-10, 6-15, 6-20, 7-10, 7-15, 7-20, 8-10, 8-15, 8-20, 9-10, 9-15, or 9-20 distinct detection molecules. In some embodiments, a detection system comprises 2, 3, 4, 5, 6, 7, 8, 9, 10, 11, 12, 13, 14, 15, 16, 17, 18, 19, or 20 distinct detectable molecules.

In some embodiments, a detectable molecule comprises an oligonucleotide that can be cleaved by the collateral activity of an effector protein. In some embodiments, a detectable molecule comprises an oligonucleotide and further comprises one or more fluorophore. Exemplary fluorophores are known to those having ordinary skill in the art. In some embodiments, a detectable molecule comprises a quenched fluorophore that exhibits fluorescence when the oligonucleotide is cleaved by the effector protein.

In some embodiments, a detectable molecule is a lateral flow reporter molecule comprising the nucleic acid sequence of 6FAM-mArArUrGrGrCmAmArArUrGrGrCmA-BIO (SEQ ID NO: 24).

F. Additional Components

A nucleic acid detection system may comprise additional components.

In some embodiments, a detection system comprises an RNase inhibitor. Exemplary RNase inhibitors are known to those having ordinary skill in the art.

In some embodiments, a detection system comprises one or more reaction buffers.

Additional components that a detection system may comprise are described in the Examples provided below.

### II. Diagnostic Devices.

In some aspects, the disclosure relates to diagnostic devices comprising: (i) one or more compartments comprising a detection system described herein; and (ii) one or more substrates.

A compartment of a diagnostic device may be a well or chamber to which a sample (e.g., potentially comprising a target molecule) may be added.

A diagnostic device may comprise multiple compartments comprising a detection system described above. For example, a diagnostic device may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 compartments. In some embodiments, a diagnostic device comprises 1-2, 1-3, 1-4, 1-5, 1-6, 1-7, 1-8, 1-9, 1-10, 2-3, 2-4, 2-5, 2-6, 2-7, 2-8, 2-9, 2-10, 2-2, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 compartments. In some embodiments, a diagnostic device comprises, 1, 2, 3, 4, 5, 6, 7, 8, 9, 10, 11, 12, 13, 14, 15, 16, 17, 18, 19, or 20 compartments.

When a diagnostic device comprises multiple compartments, the detection systems of the compartments may be the same or different. For example, in some embodiments, a diagnostic device comprises at least 2, at least 3, at least 4, at least 5, at least 6, at least 7, at least 8, at least 9, or at least 10 different detection systems. In some embodiments, a diagnostic device comprises 1-2, 1-3, 1-4, 1-5, 1-6, 1-7, 1-8, 1-9, 1-10, 2-3, 2-4, 2-5, 2-6, 2-7, 2-8, 2-9, 2-10, 2-2, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 different detection systems. In some embodiments, a diagnostic device comprises, 1, 2, 3, 4, 5, 6, 7, 8, 9, 10, 11, 12, 13, 14, 15, 16, 17, 18, 19, or 20 different detection systems. For example, in some embodiments, a diagnostic device comprises: (i) a distinct detection system for BK polyomavirus DNA; (ii) a distinct detection system for cytomegalovirus DNA; (iii) a distinct detection system for CXCL9 mRNA; (iv) a distinct detection system for CXCL10 mRNA; or (v) any combination thereof.

In some embodiments, a diagnostic device comprises a compartment comprising a detection system and a polynucleotide positive control, wherein the polynucleotide positive control comprises a nucleic acid sequence that is bound or hybridized by a guide RNA of the detection system. In some embodiments, the polynucleotide positive control comprises the nucleic acid sequence of ATGAAGAAAAGTGGTGTTCTTTTCCTCTTGGGCATCATCTTGCTGGTTCTGATTGG AGTGCAAGGAACCCCAGTAGTGAGAAAGGGTCGCTGTTCCTGCATCAGCACCAA CCAAGGGACTATCCACCTACAATCCTTGAAAGACCTTAAACAATTTGCCCCAAGC CCTTCCTGCGAGAAAATTGAAATCATTGCTACACTGAAGAATGGAGTTCAAACAT GTCTAAACCCAGATTCAGCAGATGTGAAGGAACTGATTAAAAAGTGGGAGAAAC AGGTCAGCCAAAAGAAAAAGCAAAAGAATGGGAAAAAACATCAAAAAAAGAAA GTTCTGAAAGTTCGAAAATCTCAACGTTCTCGTCAAAAGAAGACTACATAA (SEQ ID NO: 28) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 28. In some embodiments, the polynucleotide positive control comprises the nucleic acid sequence of GAAATTAATACGACTCACTATAGGATGAAGAAAAGTGGTGTTCTTTTCCTCTTGG GCATCATCTTGCTGGTTCTGATTGGAGTGCAAGGAACCCCAGTAGTGAGAAAGG GTCGCTGTTCCTGCATCAGCACCAACCAAGGGACTATCCACCTACAATCCTTGAA AGACCTTAAACAATTTGCCCCAAGCCCTTCCTGCGAGAAAATTGAAATCATTGCT ACACTGAAGAATGGAGTTCAAACATGTCTAAACCCAGATTCAGCAGATGTGAAG GAACTGATTAAAAAGTGGGAGAAACAGGTCAGCCAAAAGAAAAAGCAAAAGAA TGGGAAAAAACATCAAAAAAAGAAAGTTCTGAAAGTTCGAAAATCTCAACGTTC TCGTCAAAAGAAGACTACATAA (SEQ ID NO: 23) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 23.

In some embodiments, a diagnostic device comprises a compartment comprising a detection system and a polynucleotide negative control, wherein the polynucleotide negative control comprises a nucleic acid sequence that is not bound or hybridized by a guide RNA of the detection system.

As used herein, the term “substrate” to which a sample is applied after it has been contacted with a detection system. In some embodiments, multiple samples can be applied to a single substrate. In some embodiments, a diagnostic device comprises multiple substrates. For example, in some embodiments, a diagnostic device comprises at least 2, at least 3, at least 4, at least 5, at least 6, at least 7, at least 8, at least 9, or at least 10 distinct substrates. In some embodiments, a diagnostic device comprises 1-2, 1-3, 1-4, 1-5, 1-6, 1-7, 1-8, 1-9, 1-10, 2-3, 2-4, 2-5, 2-6, 2-7, 2-8, 2-9, 2-10, 2-2, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 distinct substrates. In some embodiments, a diagnostic device comprises, 1, 2, 3, 4, 5, 6, 7, 8, 9, 10, 11, 12, 13, 14, 15, 16, 17, 18, 19, or 20 distinct substrates.

In some embodiments, a substrate is a lateral flow strip, a flexible material substrate, a paper substrate, or a flexible polymer-based substrate.

In some embodiments, a diagnostic device further comprises an imaging component. An imaging component may be used to capture an image of a substrate (e.g., at a predetermined time following application of a sample to the substrate).

In some embodiments, a diagnostic device further comprises an image analysis component. The imaging analysis component comprises a lateral-flow quantification application as described in the Examples below.

### III. Kits.

In some aspects, the disclosure relates to kits comprising a detection system described herein or a diagnostic device comprised herein.

In some embodiments, a kit comprises a detection system described herein. A kit may comprise multiple detection systems. For example, a kit may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 detection systems. In some embodiments, a kit comprises 1-2, 1-3, 1-4, 1-5, 1-6, 1-7, 1-8, 1-9, 1-10, 2-3, 2-4, 2-5, 2-6, 2-7, 2-8, 2-9, 2-10, 2-2, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 distinct detection systems. In some embodiments, a kit comprises, 1, 2, 3, 4, 5, 6, 7, 8, 9, 10, 11, 12, 13, 14, 15, 16, 17, 18, 19, or 20 detection systems.

When a kit comprises multiple detection systems, the detection systems may be the same or different. For example, in some embodiments, a kit comprises at least 2, at least 3, at least 4, at least 5, at least 6, at least 7, at least 8, at least 9, or at least 10 different detection systems. In some embodiments, a kit comprises 1-2, 1-3, 1-4, 1-5, 1-6, 1-7, 1-8, 1-9, 1-10, 2-3, 2-4, 2-5, 2-6, 2-7, 2-8, 2-9, 2-10, 2-2, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 different detection systems. In some embodiments, a kit comprises, 1, 2, 3, 4, 5, 6, 7, 8, 9, 10, 11, 12, 13, 14, 15, 16, 17, 18, 19, or 20 different detection systems. For example, in some embodiments, a kit comprises: (i) a distinct detection system for BK polyomavirus DNA; (ii) a distinct detection system for cytomegalovirus DNA; (iii) a distinct detection system for CXCL9 mRNA; (iv) a distinct detection system for CXCL10 mRNA; or (v) any combination thereof.

In some embodiments, a kit comprises a polynucleotide positive control, wherein the polynucleotide positive control comprises a nucleic acid sequence that is bound or hybridized by a guide RNA of a detection system. In some embodiments, the polynucleotide positive control comprises the nucleic acid sequence of ATGAAGAAAAGTGGTGTTCTTTTCCTCTTGGGCATCATCTTGCTGGTTCTGATTGG AGTGCAAGGAACCCCAGTAGTGAGAAAGGGTCGCTGTTCCTGCATCAGCACCAA CCAAGGGACTATCCACCTACAATCCTTGAAAGACCTTAAACAATTTGCCCCAAGC CCTTCCTGCGAGAAAATTGAAATCATTGCTACACTGAAGAATGGAGTTCAAACAT GTCTAAACCCAGATTCAGCAGATGTGAAGGAACTGATTAAAAAGTGGGAGAAAC AGGTCAGCCAAAAGAAAAAGCAAAAGAATGGGAAAAAACATCAAAAAAAGAAA GTTCTGAAAGTTCGAAAATCTCAACGTTCTCGTCAAAAGAAGACTACATAA (SEQ ID NO: 28) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 28. In some embodiments, the polynucleotide positive control comprises the nucleic acid sequence of GAAATTAATACGACTCACTATAGGATGAAGAAAAGTGGTGTTCTTTTCCTCTTGG GCATCATCTTGCTGGTTCTGATTGGAGTGCAAGGAACCCCAGTAGTGAGAAAGG GTCGCTGTTCCTGCATCAGCACCAACCAAGGGACTATCCACCTACAATCCTTGAA AGACCTTAAACAATTTGCCCCAAGCCCTTCCTGCGAGAAAATTGAAATCATTGCT ACACTGAAGAATGGAGTTCAAACATGTCTAAACCCAGATTCAGCAGATGTGAAG GAACTGATTAAAAAGTGGGAGAAACAGGTCAGCCAAAAGAAAAAGCAAAAGAA TGGGAAAAAACATCAAAAAAAGAAAGTTCTGAAAGTTCGAAAATCTCAACGTTC TCGTCAAAAGAAGACTACATAA (SEQ ID NO: 23) or a nucleic acid sequence having at least at least 80%, at least 85%, at least 90%, at least 91%, at least 92%, at least 93%, at least 94%, at least 95%, at least 96%, at least 97%, at least 98%, at least 99% identity with SEQ ID NO: 23.

In some embodiments, a kit comprises a polynucleotide negative control, wherein the polynucleotide negative control comprises a nucleic acid sequence that is not bound or hybridized by a guide RNA of a detection system.

In some embodiments, a kit comprises a polynucleic acid isolation component that can be used to isolate polynucleic acids from a sample. In some embodiments, the polynucleic acid isolation component comprises tris(2-carboxyethyl)phosphine, EDTA, or a combination thereof. In some embodiments, the polynucleic acid isolation component comprises a purification column.

### IV. Methods of Detecting a Target Molecule in a Sample.

In some aspects, the disclosure relates to methods of detecting a target molecule in a sample. These methods may utilize a detection system described herein, a diagnostic device described herein, and/or a kit described herein. These techniques involve the SHERLOCK methodology, which has been described previously. See e.g., U.S. Pat. Nos. 10,266,886 and 10,266,887, and US Published Application Nos. 2018/0274017, 2018/0298445; 2019/0144929; 2020/0181720, the entireties of each of which are incorporated herein by reference.

In some embodiments, the method comprises: (i) contacting a sample comprising nucleic acid molecules with a detection system described herein; and (ii) confirming the presence of or the absence of a target molecule contained therein.

In some embodiments, RNAses in the sample are inhibited.

In some embodiments, the method comprises purifying polynucleotides from the sample; for example, to produce a sample comprising nucleic acid molecules (and which is contacted with the detection system).

In some embodiments, the method comprises amplifying a target molecule; for example, using an isothermal recombinase polymerase amplification method (RPA) followed by producing RNA using an RNA polymerase such as T7 RNA polymerase.

In some embodiments, the sample is a urine sample, a blood sample, a serum sample, or a plasma sample from a patient having an organ transplant. In some embodiments, the organ transplant is a renal transplant.

### V. Methods of Detecting an Opportunistic Post-Transplantation Viral Infection.

In some aspects, the disclosure relates to methods of detecting an opportunistic post-transplantation viral infection. In some embodiments, the method comprises: contacting nucleic acids from a sample obtained from a transplant patient with a detection system described herein, a diagnostic device described herein, and/or a kit described herein; wherein the target molecule is a polynucleic acid that is indicative of an opportunistic post-translational viral infection. Examples of polynucleic acid molecules that are indicative of an opportunistic post-translational viral infection are known to those having ordinary skill in the art; others are described herein. See e.g., Example Section.

In some embodiments, the method comprises detecting multiple distinct target molecules; for example, using the same detection system or using distinct detection systems.

In some embodiments, the method comprises: (i) detecting a viral DNA molecule (e.g., BK polyomavirus DNA, cytomegalovirus DNA, or a combination thereof); (ii) detecting a cytokine mRNA (e.g., CXCL9 mRNA, CXCL10 mRNA, or a combination thereof); or (iii) a combination of (i) and (ii).

In some embodiments, RNAses in the sample are inhibited.

In some embodiments, the method comprises purifying polynucleotides from the sample; for example, to produce a sample comprising nucleic acid molecules (and which is contacted with the detection system).

In some embodiments, the method comprises amplifying a target molecule; for example, using an isothermal recombinase polymerase amplification method (RPA) followed by producing RNA using an RNA polymerase such as T7 RNA polymerase.

In some embodiments, the sample is a urine sample, a blood sample, a serum sample, or a plasma sample from a patient having an organ transplant. In some embodiments, the organ transplant is a renal transplant.

### VI. Methods of Identifying a Subject Having BK Nephropathy.

In some aspects, the disclosure relates to methods for identifying a subject having a BK nephropathy (i.e., a BK virus-associated nephropathy). In some embodiments, the method comprises: contacting nucleic acids from a sample obtained from a patient with a detection system described herein, a diagnostic device described herein, or a kit described herein; wherein the target molecule is a polynucleic acid that is indicative of BK nephropathy.

Examples of polynucleic acid molecules that are indicative BK nephropathy are known to those having ordinary skill in the art; others are described herein. See e.g., Example Section. For example, in some embodiments, the target molecule is BK polyomavirus DNA.

In some embodiments, RNAses in the sample are inhibited.

In some embodiments, the method comprises purifying polynucleotides from the sample; for example, to produce a sample comprising nucleic acid molecules (and which is contacted with the detection system).

In some embodiments, the method comprises amplifying a target molecule; for example, using an isothermal recombinase polymerase amplification method (RPA) followed by producing RNA using an RNA polymerase such as T7 RNA polymerase.

In some embodiments, the sample is a urine sample, a blood sample, a serum sample, or a plasma sample from a patient.

### VII. Methods of Monitoring of Transplant Rejection.

In some aspects, the disclosure relates to methods for the monitoring of transplant rejection. In some embodiments, the method comprises: contacting nucleic acids from a sample obtained from a transplant patient with a detection system described herein, a diagnostic device described herein, or a kit described herein; wherein the target molecule is a polynucleic acid that is indicative of transplant rejection.

Examples of polynucleic acid molecules that are indicative of transplant rejection are known to those having ordinary skill in the art; others are described herein. See e.g., Example Section.

In some embodiments, the method comprises detection multiple distinct target molecules; for example, using the same detection system or using distinct detection systems. In some embodiments, the method comprises: (i) detecting a viral DNA molecule (e.g., BK polyomavirus DNA, cytomegalovirus DNA, or a combination thereof); (ii) detecting a cytokine mRNA (e.g., CXCL9 mRNA, CXCL10 mRNA, or a combination thereof); or (iii) a combination of (i) and (ii).

In some embodiments, the target molecule is a cytokine mRNA (e.g., CXCL9 mRNA or CXCL10 mRNA).

In some embodiments, RNAses in the sample are inhibited.

In some embodiments, the method comprises purifying polynucleotides from the sample; for example, to produce a sample comprising nucleic acid molecules (and which is contacted with the detection system).

In some embodiments, the method comprises amplifying a target molecule; for example, using an isothermal recombinase polymerase amplification method (RPA) followed by producing RNA using an RNA polymerase such as T7 RNA polymerase.

In some embodiments, the sample is a urine sample, a blood sample, a serum sample, or a plasma sample from a patient having an organ transplant. In some embodiments, the organ transplant is a renal transplant.

### EXAMPLES

### Example 1: Introduction to Specific Aims of Examples 2-5

More than 50% of kidney transplants fail by 10 years after transplant and minimal improvement in long-term graft survival has been achieved in the past 30 years (1-3). This translates into a large proportion of failed kidney transplant patients going back to dialysis, which has an annual budget of 37 billion dollars in the USA. An unmet need in kidney transplantation is the ability to identify allograft injury non-invasively, allowing more frequent testing, earlier diagnosis and treatment. This is particularly relevant since serum creatinine, the assay currently used to monitor graft function, is nonspecific, not sensitive and a delayed marker that may detect graft injury only after significant injury has occurred. Indeed, about a third of patients have already developed significant kidney scarring by the time creatinine is elevated, missing an important window for treatment.

Rejection and BK nephropathy are leading causes of long-term graft loss (4). While a few promising biomarkers have emerged such as CXCL9 and CXCL10 (5-13), a cheap, fast and sensitive point-of-care assay to monitor graft injury is lacking. Furthermore, multiple biomarkers are likely to be needed to capture various causes of graft injury and, consequently, to better guide clinical decisions. Lastly, low-cost, non-invasive, frequent testing would be ideal to bring precision medicine to the forefront of transplantation.

CRISPR-based diagnostic techniques have been shown to successfully detect DNA viruses, RNA viruses, bacteria and cell-free DNA in urine, blood, and saliva (14-17). Although these proof of concept studies clearly show the high potential of this diagnostic approach, they have not been translated to clinical application yet. In the studies described herein, CRISPR/Cas13 was used to detect urine BK virus (BKV) DNA and CXCL9/CXCL10 mRNA. Preliminary studies show detection of BKV DNA down to 10 aM or 6,000 copies/mL within 1 hour of fresh or frozen urine samples processing. This detection level was confirmed with the use of a pregnancy-like test strip. CXCL9 mRNA detection is also achievable with great sensitivity requiring solely RNA later treated urine to prevent mRNA degradation. Therefore, it is feasible to quickly detect crucial biomarkers in kidney transplantation with this low-cost technique. The latter is a crucial factor for broad applicability as many countries, including Brazil, do not routinely perform BK surveillance due largely to financial restrictions (U$30/assay not covered by the Brazilian Public Health System), despite recommendations of transplant guidelines.

The objective of this study is to develop a point-of-care diagnostic test to detect BKV infection and kidney allograft rejection using Sherlock (specific high-sensitivity enzymatic reporter unlocking), a novel diagnostic platform of CRISPR/Cas13 technology. It was hypothesized that detection of BKV DNA and chemokines mRNA (CXCL9 and CXCL10) can be achieved in the urine of kidney transplant patients using a simple isothermal amplification technique combined with specific CRISPR/Cas13 detection for the relevant biomarkers. The advantage of this technique involves the multiplexed potential, low cost (below U$5), lack of need of ancillary equipment and possibility to obtain results within 1 hour of sample collection.

Aim 1. To optimize the diagnostic CRISPR/Cas13 assay for specific transplant biomarkers. The hypothesis that BKV DNA and selected chemokines mRNA can be reliably detected with high sensitivity and specificity in urine and plasma will be tested under various processing conditions and concentrations using recombinase polymerase amplification and CRISPR/Cas13 in comparison to gold standard (RT)-PCR. A fast lateral-flow readout will be optimized, which produces a visible output similar to a pregnancy strip test. Urine and plasma from healthy volunteers will be spiked with BKV DNA or chemokine mRNA for this aim (conducted in the USA).
Aim 2. To validate the CRISPR/Cas13 assay in retrospective biobanked samples of patients with rejection, BK nephropathy and stable patients. The hypothesis that the CRISPR/Cas13 assay is capable of identifying rejection or BK nephropathy cases in biobanked samples stored at time of clinically-indicated kidney biopsy, comparing with gold standard will be tested. This aim will be conducted both in the USA and in Brazil.
Aim 3. To assess and validate the CRISPR/Cas13 biomarker assay in prospective samples collected from patients post-transplantation in two transplant centers in the USA and Brazil. The hypothesis that persistent elevation of urine BKV DNA and/or chemokines precedes the elevation in creatinine in kidney transplant recipients, allowing earlier diagnosis of graft injury will be tested.
Impact: This proposal will develop a novel multiplexed biomarker assay to improve monitoring of kidney transplant patients at point-of-care. The high sensitivity and low-cost of this assay will be particularly impactful in Brazil and in developing countries where most centers currently do not perform BKV surveillance for financial reasons despite supportive guidelines. Finally, this work will have an impact beyond the transplant field, since this technology could be easily adapted for the monitoring of immune-mediated kidney disease in non-transplant patients.

### Significance:

Rejection and BK nephropathy are major causes of long-term kidney graft loss: There are more than 20,000 new solid organ transplants per year in the USA and more than 50% of grafts fail by 10 years after transplant, significantly reducing patient survival, inflating the deceased donor kidney waitlist and increasing healthcare costs with dialysis that surpasses 35 billion dollars per year (1). Long-term graft survival has not significantly improved in the past 30 years (3) and immunological-related graft injury remains a major cause of chronic graft loss (4). Specifically, in patients that survive up to 5 years post-transplant, rejection and BK nephropathy are leading causes of interstitial fibrosis and long-term graft loss (4). Unfortunately, about ⅓ of patients have already developed significant kidney scarring by the time creatinine is elevated, missing an important window for treatment and prevention of irreversible injury.

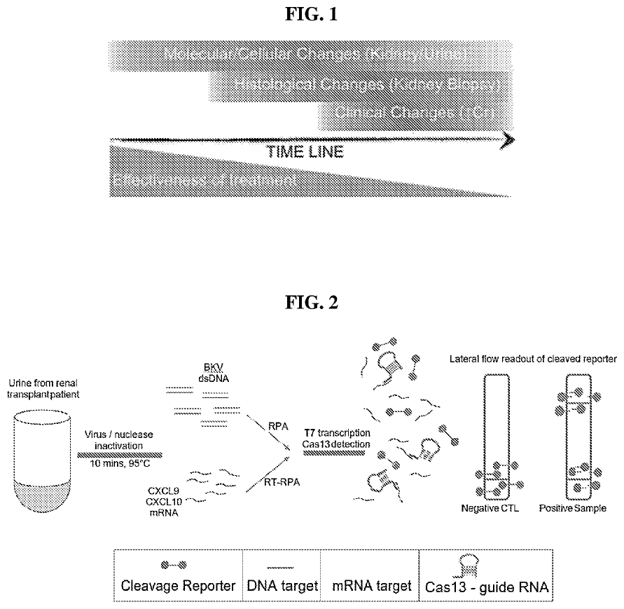

Serum creatinine is a delayed marker of graft injury: An unmet need in kidney transplantation is the ability to identify rejection non-invasively, allowing more frequent testing, earlier diagnosis and treatment of rejection. This is particularly relevant since serum creatinine, the current monitoring assay to suspect rejection, is a non-sensitive and delayed marker of graft injury (FIG. 1). Furthermore, the diagnosis of acute rejection or BK nephropathy currently requires a renal biopsy—an invasive process that is limited by sampling error and assessment variability. In order to earlier detect graft injury, some centers perform surveillance kidney biopsies at prespecified time-points post-transplant. These biopsies lead to change in management in ˜25% of cases depending on the population studied (18). However, these procedures are associated with significant risks for patients (e.g., bleeding) and cost (˜$2,500/biopsy, which includes the procedure and the pathological analyses of the kidney specimen). As an example, USP-Brazil transplant center follows 2,108 patients in their outpatient clinic and the performance of protocol biopsies in such large populations is logistically impossible. In the US, less than 40% of transplant centers perform surveillance biopsies (19). Therefore, more sensitive and non-invasive tools to detect graft injury may allow more effective interventions and likely improve long-term graft survival. Among those, one of the most promising biomarkers under investigation are the urinary chemokines CXCL9 and CXCL10.

Urinary chemokines (CXCL9/CXCL10) are promising transplant biomarkers of rejection: Chemokines are crucial signals that direct immune cells to specific sites of inflammation for an optimal immune response (20, 21). The production of IFN-γ at the inflammatory site by memory T cells, natural killer cells and other innate cells stimulates the production of IFN-γ-induced chemokines such as CXCL9 and CXCL10, leading to the recruitment of CXCR3+ effector T cells. These two cytokines have been shown to be crucial in mediating acute and chronic graft injury (22, 23). Several studies have indicated that the presence of high levels of urinary CXCL9 and/or CXCL10, assessed either by protein or mRNA levels, are able to differentiate acute rejection from most other causes of graft dysfunction with exception of infection (5-13, 24-26). A prospective multicenter study of 280 kidney transplant recipients reported that urinary CXCL9 mRNA levels had an AUC=0.789 to detect T-cell mediated rejection with an odds ratio of 2.77 (1.59-4.8, p=0.0003). Furthermore, urinary CXCL9 levels were elevated ˜30 days prior to clinical detection of acute rejection, indicating that it could serve as an earlier marker for rejection (7, 8). Furthermore, others have identified an association between urinary CXCL10 levels with the diagnosis of antibody-mediated rejection (ABMR) (13). Indeed, urinary CXCL10 levels identified ABMR (AUC=0.76; 95% CI, 0.69 to 0.82; P, 0.001) with high accuracy, reflecting peritubular capillaritis (27). Elevation of CXCL9 and CXCL10 has also been shown to detect subclinical rejection on protocol biopsies (6,24), supporting the hypothesis that monitoring of urine chemokine levels, even in clinically stable kidney transplant recipients (no changes in creatinine levels), might provide additional information for assessing allograft status. Lastly, low CXCL10 levels at 1 and 3 months post-transplant, and less robustly CXCL9, were associated with rejection-free survival at 1 year (28). In sum, these studies suggest that both cytokines are strong candidates for biomarkers of rejection with the potential to identify both types of rejection and predict future risk of rejection.

BK nephropathy is an important differential diagnosis of acute graft dysfunction and a marker of overimmunosuppression: BK nephropathy is major cause of graft loss with 30 to 65% of patients that develop BK-associated nephropathy lose their kidney allograft within 1 year of diagnosis (29). Guidelines from the American Society of Transplantation recommends performance of serial blood or urine BK viral load measurements in the first-year post-transplant in order to earlier detect BK replication and reduce immunosuppression (29,30). However, the significant cost involved limits its broad application worldwide. As an example, in Brazil, more than 90% of the transplant centers do not perform BK surveillance due to financial constraints. For example, each PCR for the detection of BK virus cost U$30 in Brazil and it is not reimbursed by the Universal Brazilian Health Care System that covers all transplant-related costs. Although BK infection is mostly prevalent in the first-year post-transplant, late BK nephropathy is diagnosed in about 10% of kidney recipients after the second year of transplant, without previous evidence of viral replication (31), supporting the screening for BK beyond the first of transplant. This is particularly important since reduction of immunosuppression is the only intervention that consistently impacts the course of BK infection, preventing the development of significant BK nephropathy (29). There are no antiviral agents that effectively treat BK infection at this time. A low-cost, fast assay would increase the detection of BK infection, allowing more frequent testing and earlier intervention with timely reduction of the immunosuppression. Furthermore, differentiating allograft dysfunction from rejection or BK infection is crucial due to the opposite management strategies. Reduction of immunosuppression after BK detection leads to rejection in more than 50% of transplanted patients (31). Therefore, the availability of a differential point-of-care assay with frequent monitoring may significantly improve clinical care.

### Innovation:

Described herein is a point-of-care diagnostic test to detect BK virus infection and kidney allograft rejection (FIG. 2). Both require frequent testing which until to date depends on extensive diagnostic equipment, leading to high costs and slow turn-over time. This fast (˜1 hour) and cost-effective (<$5) new diagnostic tool will combine isothermal amplification and a CRISPR (short for Clustered Regularly Interspaced Short Palindromic Repeats)-based method (detailed below) to detect viral BK DNA and specific chemokines' mRNAs indicative of rejection. This novel technique has been shown previously to successfully detect DNA viruses, RNA viruses, bacteria and cell free DNA in urine, blood and saliva (14-17). Although these proof of concept studies could clearly show the great potential of CRISPR-based diagnostics, they have not been translated to clinical application yet. Further, the detection of mRNA biomarkers with this technique has not been reported.

The diagnostic test, which combines detection of infection (BK virus) and rejection (CXCL9 and CXCL10 mRNA biomarkers), aims to pave the way for CRISPR diagnostics in the clinic by enabling fast decision making with high sensitivity. As the test will be optimized for a point-of-care and potentially in the future as a home test, it enables better surveillance of opportunistic infection and transplant rejection potentially leading to prolonged graft survival. Importantly, the low costs enable testing in resource-limited settings and provide fast diagnostics in areas distant from major clinical centers.

### Feasibility:

Since the initial report of CRISPR diagnostics, many further advances have been made due to the high potential of the technique in a variety of research fields. Recent progress includes increased sensitivity down to the attomolar range, single-reaction multiplexing, quantitative measurements and a point-of-care suitable lateral flow readout. This platform has been shown to robustly detect DNA and RNA in urine, blood and saliva, which provides diagnostic potential of the technique in the clinical setting. As the measurement of the DNA copy number by qPCR is an established clinical readout for BK virus load, it is highly likely that the novel CRISPR based assay can robustly inform on BKV replication. Preliminary tests have confirmed the stability of BKV detection under various conditions including freezing and longer incubation times of urine. Regarding rejection, CXCL9 and CXCL10 mRNA in the urine have emerged as potential biomarkers to provide relevant information about acute kidney rejection in multicenter studies, though reliable and low-cost assays are lacking. Preliminary data supports the detection of CXCL9 mRNA in urine with great sensitivity, especially when combined with amplification methods. Furthermore, standardization of urine processing for mRNA extraction has been optimized and defined in a multicenter study of the Clinical Trials in Organ Transplantation (32), demonstrating strong correlation, reproducibility and consistency among six centers.

### Collaboration, Unique Features of Individual Centers and Impact:

The overall aim of this project is to test a non-invasive tool for the diagnosis of rejection and BK infection. The success of this project will impact how kidney allograft monitoring is performed worldwide. For this proposal, the BWH-USA team will be responsible for optimizing the CRISPR/Cas13 assay and test it in their own cohort of patients both retrospectively as well as prospectively. The USP-Brazil team will apply the assay to their large population of transplant patients including those undergoing kidney biopsy as well as those followed prospectively during the first-year post-transplant, serving as a validation cohort.

TABLE 1
Transplantation centers and annual biopsy numbers.

| | Kidney | Kidney |
|---|---|---|
| | Biopsies | Biopsies |
| Transplant Center | 2017 | 2018 |
| BWH-USA | 105 | 95 |
| USP-Brazil | 301 | 280 |

The Brigham and Women's Hospital (BWH) is a leading academic institution in the US and the location of the first kidney transplant in the world in 1954. The Transplant Research Center (TRC) is a collaborative enterprise between BWH and Harvard Medical School in Boston. The principal goal of the center is to understand the immune mechanisms of transplant rejection and develop novel biomarkers to improve transplant outcomes. Since 2011, TRC has established a biobank of samples from transplanted patients in order to better monitor and guide immunosuppression adjustment post-transplantation (225 patients recruited as of February 2019 with 920 biobanked urine and plasma samples). The intellectual and scientific environment at TRC as well as the resources and infrastructure for both basic immunobiology and translational science are unparalleled. The strong interaction with other investigators such as Jim Collins, Termeer Professor of Medical Engineering and Science at MIT, had permitted a cross-disciplinary collaboration, leading to the current proposal that explores a novel biomarker technology tailored to improve the management of kidney transplant patients. However, in order for a biomarker study to be impactful, it must be validated in large transplant centers with greater heterogeneity, ensuring the generalizability of the results.

The Hospital das Clínicas/USP is a leading academic center in Brazil and performs 200-220 kidney transplants every year. Currently, USP transplant center is one of the few centers in the country that performs BK surveillance post-transplantation (covered by the university hospital). Furthermore, the Brazilian population that is transplanted in this center is composed of mixed race Mulatos (mixed White-African American) (45%), Afro-Brazilians (8%), Europeans (30%) and Asians (1%), overall very different from the US population. As a comparison, the BWH transplant recipients are composed of 65% White, 21% African Americans, 9% Hispanics and 4% Asians. Lastly, differences in immunosuppression between centers (detailed below) reflect a known heterogeneity in management of transplant recipients worldwide, which generates an important diversity when combining both centers' results. Overall, if the biomarker findings in these two different populations show similarities, then the results could be then extended to almost all populations. Since the remaining transplant centers in Brazil do not monitor for BK infection due to costs, the introduction of a novel test with low cost and fast turnaround for BK infection will be of great interest for countries like Brazil. Furthermore, the ability to monitor for rejection with the chemokines' assessment further increases the potential of this tool in improving transplant patient care and earlier diagnose rejection or BK infection.

These two centers have already developed a collaborative study on the recurrence of glomerular disease post-transplantation (www.tangoxstudy.com). Through bimonthly email updates, video conferencing (every 3 months) and in-person meetings at the American Transplant Congress (Seattle 2018, Chicago 2017), an effective approach has been developed to communication, having one responsible person from each center taking the lead. These forms of communication have allowed one to discuss cases, clarify/resolve problems and make consensus decisions in the TANGO study, leading to the publication of this study protocol this past year (33) and another original research publication in preparation exploring FSGS recurrence post-transplantation. Based on its success, a similar approach will be applied to this proposed project.

Clinical data will be collected in a de-identified manner using Redcap (Research Electronic Data Capture), a browser-based, metadata-driven electronic data capture software solution, for designing clinical and translational research databases (projectredcap.org). Investigators have access to the secure website for entering and accessing patient data online, which will be stored at a secure and confidential location. Individual centers have access to their own recorded data that they can use for analysis. In the prospective phase of the study (Aim 3), the BWH/USA site will be blinded to the clinical outcomes in the initial analyses in order to avoid any potential bias in the interpretation of results. All clinical data is de-identified across the centers in order to protect patients. Shipping of samples have been successfully performed in the current collaborative TANGO study, in which over 50 samples were sent from Brazil to Boston. Briefly, the study will be registered at the National Ethics Committee (CONEP) through Plataforma Brasil and at local IRB at the BWH, with a material transfer agreement signed between institutions. Information about the samples will be shared to the Brazilian Health Vigilance Agency (ANVISA) and a letter will be included describing the samples being shipped according to CDC US guidelines. USP will store samples in a −80° C. freezers (duplicates of samples will be stored in two different freezers) with the plan to ship one aliquot in batches when all documents are approved. Training of staff in Brazil will also be performed on Aim 3 to allow performance of the assay using lateral flow readout locally in Brazil without the need to ship all samples to the USA. Lastly, a research candidate in Brazil will be selected to spend 3 months in the Transplant Research Center to transfer knowledge and expertise to the Brazilian collaborator's group.

### Example 2. Specific Aim 1. Optimize Diagnostic CRISPR/Cas13 Assay for the Detection of Specific Transplant Biomarkers

Rationale: It is crucial to develop a highly sensitive and accurate diagnostic assay with low-cost in order to permit broad applicability of this this technology. In this aim, a CRISPR/Cas13 assay will be optimized for the detection of BKV DNA and CXCL9/CXCL10 mRNA under various urine processing conditions and DNA/mRNA concentrations, comparing to the gold standard (RT)-PCR.

### Preliminary Data:

The diagnostic assay combining Recombinase Polymerase Amplification (RPA) and CRISPR/Cas 13 is termed SHERLOCK and involves 3 major steps: nuclease inactivation through a short heating step (10 min), an isothermal amplifying step called RPA coupled with T7 transcription, and the Cas13 detection using cleavage reporters (FIG. 2) (14, 15). RPA, in contrast to PCR, does not require cycling and extensive laboratory equipment. The read-out of the assay can then be done by measuring fluorescence output in a fluorescent plate or with the use of a lateral-flow readout detailed below.

Selection of BK primer: A conserved region of the BK virus genome was selected as a target for the RPA primers used to amplify BK virus DNA prior to T7 transcription and Cas13 detection.

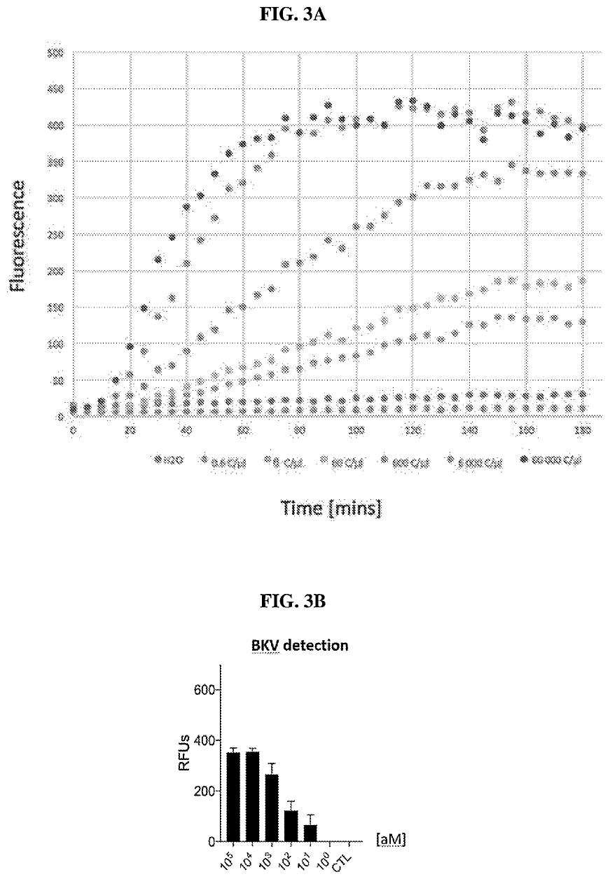

Detection of BK virus using CRISPR/Cas13 assay: Different primers were tested using the diagnostic quantitative standard (synthetic BK virus DNA obtained from ATCC). Optimization of the primer design and reaction conditions resulted in robust detection of the synthetic standard down to the single attomolar range (˜1000 Copies/ml) (FIG. 3).

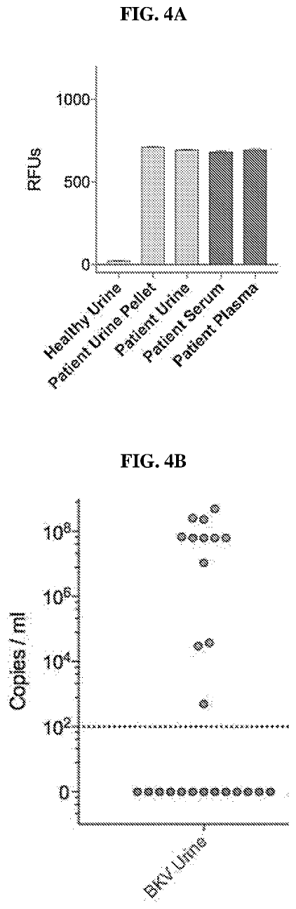

Next, different point-of-care-compatible virus inactivation and isolation protocols were tested. Similar to previous studies (34), it was found that short heating to 95 degrees Celsius for 10 minutes in presence of 1 mM EDTA and 100 mM TCEP was effective to inactivate RNAses and lyse the virus. This enabled fast and sensitive detection of BKV in urine, serum and plasma (FIG. 4A).

In a different set of experiments, the performance of the diagnostic was tested on multiple patient samples from the diagnostic core with known BK virus copy numbers as measured by the current gold standard qPCR. The results showed 100% sensitivity and specificity in detecting BK virus DNA in patient's urine as compared to the PCR results (FIG. 4B).

Selection of CXCL9 primer: CXCL9 specific primers for RT-RPA were selected based on published qPCR primers used in a clinical multicenter study (7). Primers and guide RNAs detecting the amplified region were tested on synthetic CXCL9 standards.

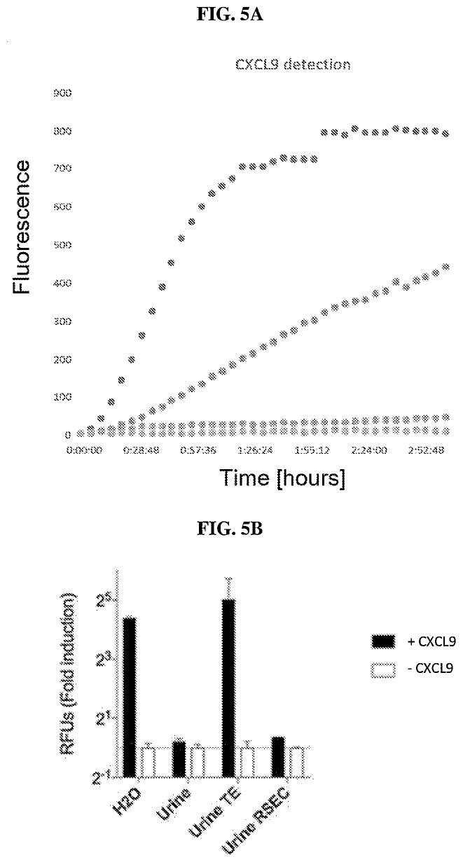

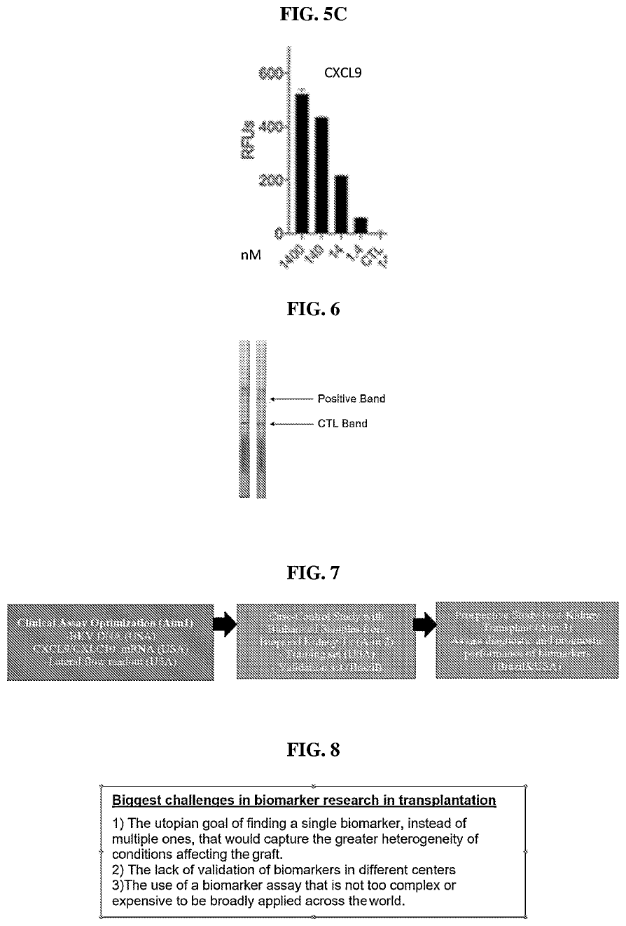

Detection of CXCL9 mRNA using CRISPR/cas13 assay: Different RNAse inactivation protocols that enabled preservation and following detection of the mRNA were tested (FIG. 5A). Using a similar protocol as described above, mRNAs was detected down to the low nanomolar range using CRISPR/Cas13 only (FIG. 5B) and down to the femtomolar range using a combination of RT RPA and CRISPR/Cas13 (FIG. 5C).

Development of CXCL10 mRNA assay, determine its range of detection and coefficient of variation. Similar to CXCL9, CXCL10 specific primers for RT-RPA will be selected and then urine from healthy controls will be pooled, aliquoted and spiked with CXCL10 chemokine mRNA, followed by sequential dilutions, so concentration in each sample is known. Urine will then be transferred to reaction vessels in a biosafety cabinet and heated to 95 degrees Celsius for 10 minutes in presence of 1 mM EDTA and 100 mM TCEP to inactivate RNAses and lyse virus as described before (17). Studies will be done to test if CRISPR/Cas13 is sufficient to detect mRNAs present in the urine without amplification. If this fails, it will be combined with RT-RPA. In brief, one μl of inactivated sample will be transferred to nucleic acid amplification using sequence specific primers. Reverse transcription Recombinase Polymerase Amplification (RT-RPA) will be used to amplify the conserved sequence of CXCL10. The amplification product from RT-RPA will be T7 transcribed to RNA and then incubated with a CRISPR guide RNA directing Cas13 to its target triggering cleavage of reporter molecules. Fluorescence emitted from the cleaved reported will finally be detected using a fluorescent plate reader. The CXCL10 dilution experiment will permit us to determine the cut-offs of detection of the assay when compared to gold-standard RT-PCR. The ultimate goal is to develop a semi-quantitative read-out for robust detection of clinically relevant cut-offs.

The samples will be tested by CRISPR/Cas13 technique to establish a standard curve to determine the detection range of the assay. Cutoff levels for “positive” vs. “negative” tests will be determined that can be detected when compared with gold-started RT-PCR.

Determining Best Method for mRNA Preservation and Sensitivity Taking into Account a Simplified Approach:

Rationale: Although the standardization of urine processing for mRNA extraction has been optimized and defined in a multicenter study of the Clinical Trials in Organ Transplantation (32), studies will be performed to assess if this fast technique would allow further simplification of the sample processing. The impact of freezing/thawing cycles on assay performance will also be tested.

To examine the effects of variation in sample processing, mRNA chemokine-spiked urine will be tested under the following processing conditions, followed by CRISPR/Cas13 assay:
Fresh urine+synthetic CXCL9+RNAlater (100 ul of urine with 200 ul of RNAlater)->tested
Fresh urine+synthetic CXCL9->spun down and pellet resuspended in RNAlater (100 ul)->tested
Fresh urine+synthetic CXCL9+RNAlater->frozen->thaw->tested
Fresh urine+synthetic CXCL9->spun down and pellet resuspended in RNAlater and freeze->thaw->tested.

Up to three freeze/thaw cycles will be performed to measure variability. The coefficient of variation will then be calculated on the quantities reported for each target molecule.

Optimization of Lateral Flow Readout for Point-of-Care Results:

Rationale: Developing an assay that could be performed at point-of-care would significantly impact its clinical applicability. Therefore, once the fluorescence-based readout has been established, a one-pot reaction with a lateral flow readout that involves the use of paper spotting and lyophilization (Milenia HybriDetect) will be developed (15,17). Preliminary data demonstrates that a lateral flow assay together with a FAM/Biotin reporter could enable fast and easy identification of BK virus positive patient samples (FIG. 6). In this sub-aim, this readout for BKV DNA will be optimized and extended to detect CXCL9/CXCL10 mRNA.

First, the limit of detection will be assessed by using BKV DNA at different concentrations. Similar experiments will be performed with CXCL9 and CXCL10 mRNA. Different cleavage reporters will then be tested and the concentration of Cas13 and cleavage reporters used will be optimized. Lastly, the sensitivity will be compared to the gold standard (RT)-PCR.

### Potential Pitfalls and Alternative Approaches

Although the preliminary data is strong to support BKV DNA detection in the urine, mRNA detection using CRISPR-Cas13 may not be as sensitive in the urine compared to blood, in part related to mRNA degradation (35). One of the advantages of the CRISPR/Cas13 technology is that it can be adapted to different targets and samples (urine vs blood). Therefore, if any issues of sensitivity are encountered with the rejection markers in the urine, one could quickly assess the same CRISPR-Cas13 platform for CXCL9/CXCL10 mRNA in blood plasma samples (biobanked in the center), since mRNA has greater stability in blood compared to urine. Furthermore, cell-free donor-derived DNA (Cfd DNA) detection in the recipient's blood has shown great potential to detect allograft injury after heart and kidney transplantation (36, 37). Briefly, injury of the allograft independent of the cause may lead to the release into the circulation of Cfd DNA. However, the price tag of U$2,200 per commercial test prevents any broad applicability of this assay in transplantation worldwide. Since HLA characterization of both donors and recipients is routinely performed in transplantation and cell free DNA detection has already been performed using the CRISPR/Cas13 platform, the development of specific DNA targets of certain HLA donor specificity may permit an individualized monitoring of allograft damage. Therefore, as an alternative approach, one could also consider developing the CRISPR/Cas13 assay to detect specific donor cell-free DNA targets in the plasma or urine to allow identification of graft injury independent of the cause, broadening the applicability of the technology. Detection of cell-free DNA would even be capable of detecting other less common causes of graft injury, such as drug toxicity (e.g., calcineurin inhibitor tubular toxicity or allergic interstitial nephritis) that may ultimately impact graft survival.

### Example 3. Specific Aim 2. To Validate the CRISPR/Cas13 Assay in Retrospective Bio-Banked Samples of Patients with Rejection, BK Nephropathy and Stable Patients and to Determine Test Characteristics

Rationale: The development of biomarkers that can earlier diagnose graft injury such as rejection or BK nephropathy are critically needed to allow earlier intervention and prevent irreversible graft damage. In this aim, the assay will be tested for urinary BK and chemokines detection in well-characterized samples taken at the time of clinically-indicated biopsies for acute graft dysfunction.

Training set: 72 post-transplant urine samples taken at time of kidney biopsy will be obtained from the biobank at the Transplantation Research Center (protocol approved by the ethical committee of the Partners Human Research Committee (PHRC) at the Brigham and Women's hospital in Boston, protocol number 2017P000298). Samples will be selected based on diagnosis of the biopsy report, and categorized into acute cellular rejection (n=12), acute antibody-mediated rejection (n=12), acute borderline rejection (n=12), normal biopsy (n=12), BK-nephropathy (n=12) and healthy controls (n=12). All samples will be analyzed using both CRISPR/cas13 assay and RT-PCR, to assess whether CRISPR/cas13 can equal gold standard tests.

Validation set: Another 120 samples will be obtained by the Brazilian center for validation of the findings, which will include 20 samples on each of the groups above that will be tested by both CRISPR/cas13 assay and RT-PCR. Lastly, further validation on prospective samples will be performed as described on aim 3.

### Immunosuppression

BWH-USA: Two induction agents are used for high (anti-thymocyte globulin) or low immunological risk (basiliximab) patients, according to age, history of chronic infection, prior cancer and presence of donor-specific antibodies. Standard maintenance immunosuppressive regimen consists of tacrolimus, mycophenolate mofetil (MMF) and prednisone, with steroid withdrawal in about 60% of patients long-term (low immunological risk).

USP-Brazil: Similar immunosuppression protocol is used in Brazil compared to the BWH with the exception that all patients are continued long-term on steroids. This unique difference is an important strength of this study since reflects the ongoing worldwide debate about potentials risks and benefits of steroids withdrawal post-transplantation.

### Coefficient of Variability

Monitoring of assay variations over time. Analytical errors, that include the run-to-run variations in accuracy and precision, will be monitored. There are two components of analytical errors: random error and systematic error. Random error occurs as a result of erratic run-to-run variations in assay method. Systematic error leads to positive or negative bias in assay results and is often the result of effects such as deterioration of reagents or controls or drift in calibration of the instruments. To track systematic error that might develop over multiple runs over a period of time, two reference controls will be run on each assay and the QC rules developed by Westgard and colleagues will be executed (41,42).

### Statistical Analyses

BKV detection: Since the assay will be used as a binary screening-assay (negative/positive outcome) to guide further testing, the required sample size was calculated to determine sensitivity and specificity of the CRISPR/cas13 test for BK nephropathy in the biobanked samples. Although preliminary data in few samples resulted in a sensitivity and specificity of the test of 100% (FIG. 4B), experiments were designed to test the hypothesis whether the CRISPR/Cas13 test is useful as a screening assay, e.g., a null hypothesis that the sensitivity of the test equals 50%, and an alternative hypothesis that the test has a sensitivity of at least 90%. Samples will be selected on positivity/negativity to obtain equal group size (prevalence of 50%). To test with a type I error of 0.05 and power above 80%, 12 samples will be needed in each group and a total sample size of 24 urine samples (39).

CXCL9/CXCL10 detection: Outcomes of CXCL9/CXCL10 will be semi-quantitative in RFUs as shown in FIG. 5B and will be calculated to concentrations by using a standard curve. To assess agreement between CRISPR/Cas13 technique and the gold standard (PCR), a Bland-Altman plot will be used. This plot is widely known for comparing new assays with established ones and visualizes how the difference between the two measurements varies with the average of the two measurements (40). It can also reveal trends in agreement at different concentrations, which is important in this assay since the agreement at lower concentrations is more pivotal than with very high levels of CXCL9/CXCL10. If agreement is confirmed, correlation of levels of CXCL9 and CXCL10 with state of rejection or BK nephropathy will be assessed by multivariable linear regression. An empirical ROC-curve will be constructed and area under the curve calculated to measure diagnostic accuracy. A cutoff with highest Youden index (highest sensitivity and specificity) will be determined.

### Potential Pitfalls and Alternative Approaches

Preliminary data demonstrates that CRISPR/cas13 assay is very sensitive to detect BK DNA in the urine and serum/plasma. Furthermore, urine BK DNA is stable under various conditions. Even though BKV DNA in urine or plasma is the gold-standard test for monitoring for BK infection, it is possible that false positive results may occur in a subset of patients based on the ubiquitous nature of BK virus, identifying patients with latent instead of active infections (43-45). An alternative approach would be to measure a BK specific mRNA such as VP1 (46,47), which is contingent upon BK virus DNA replication. Therefore, it may allow to better detect active infections involving the kidney transplant. Since samples are already being processed for chemokine mRNA, it would be feasible to adapt these technique to measure VP1 mRNA for BK, using a highly conserved region among major subtypes of BK virus if false positive results are high on the training set (45, 48). Another alternative would be to perform blood detection of BK virus using the plasma already biobanked. Blood BK virus has been shown to have a higher correlation to BK nephropathy though it would limit the point-of-care approach primarily proposed in this grant since it will require a blood collection. Despite that, future development of the technique could allow the use of smaller amounts of blood such as a drop of blood.

### Example 4. Specific Aim 3. To Assess and Validate the CRISPR/Cas13 Biomarker Assay in Prospective Samples Collected from Patients' Post-Transplantation in Two Transplant Centers in the US and Brazil

Rationale: The diagnostic performance of a biomarker, in particular, its positive and negative predictive value, depends on the prevalence of the outcome, e.g., BK infection or rejection. Therefore, assessing the diagnostic values in real-life settings with a representative prevalence of the disease is crucial (49,50). These studies will focus on 2 high-risk groups: first year post-transplantation or patients who had their immunosuppression reduced by more than 50%. This is crucial since if one extrapolates the literature of cardiac biomarkers, such as troponin, the highest yield for a biomarker is encountered when applied to the greatest risk population (e.g., Troponin in a patient with chest pain). Furthermore, confirmation of the findings in an independent and representative patient cohort is important. In this aim, the hypothesis that persistent elevation of urine BK DNA and/or chemokines precedes the elevation in creatinine in kidney recipients will be tested, which would allow earlier diagnosis of graft injury. To test this hypothesis, prospectively urine specimens will be collected from kidney transplant recipients in two cohorts (BWH and USP), examining BK DNA and CXCL9/CXCL10 mRNA in combination with routine clinical care and surveillance biopsy at 6 months post-transplant. It is anticipated that a total of ˜450 patients will enroll in a year including both centers (130 in the USA and 320 in Brazil).

### Transplant Cohorts

Patients who received a kidney transplantation will be recruited at the Brigham and Women's Hospital (USA) and at USP (Brazil). Each center performs in average 70 and 200 transplants per year. Both centers will monitor patients according to their usual standard of care. USP is one of the few centers in Brazil that perform BK surveillance in the blood, and they will continue to perform those during the study period as recommended by American Society of Transplantation guidelines. In order to assess high-risk groups for rejection, these studies will enroll any transplanted patient that had the immunosuppression reduced by more than 50% post-transplantation. Specifically, patients with mycophenolate mofetil below 750 mg daily and tacrolimus trough levels below 4 based on the risk associated with donor-specific anti-HLA antibody development, rejection and graft loss will be enrolled (51).

TABLE 2
Transplantation centers and annual statistics.

| | | | Rejection | BK infection | |
|---|---|---|---|---|---|
| Transplant | Kidney | Kidney | 1^{st} year | 1^{st} year | Long-term |
| Center | 2017 | 2018 | (2018) | (2018) | kidney tx pts |
| BWH-USA | 67 | 76 | 14 (18%) | 12 (16%) | 954 |
| USP-Brazil | 210 | 202 | 30 (15%) | 36 (18%) | 2,108 |

Inclusion Criteria

Patients who meet all of the following criteria are eligible for enrollment as study subjects:

1. Subject must be able to understand and provide written informed consent;
2. Male or female, 18 years of age and older;
3. Kidney transplant recipients within first year of transplant or kidney transplant recipients at any time post-transplant that had the immunosuppression reduced by more than 50% due to any cause.

Exclusion Criteria

Patients who meet any of these criteria are not eligible for enrollment as study subjects:
1. Recipient of multiple organ transplants;
2. Any condition that would preclude protocol biopsies;
3. Any condition that, in the opinion of the investigator, would interfere with the subject's ability to comply with study requirements;
4. Inability or unwillingness of a subject to comply with study protocol.
5. Primary graft non-function
6. Patients with severe anemia (hematocrit <21%)

### Samples' Collection

Urine from consented patients will be collected at the time of scheduled clinical visits at months 1, 2, 3, 4, 6, 9, and 12 post-recruitment; or at the time of each clinically-indicated kidney allograft biopsy (including surveillance biopsy at 6 months post-transplant). Two follow-up samples will be collected within 8 weeks of the kidney allograft biopsy in order to assess the dynamic of changes close to biopsy performance. Duplicate samples of urine will be collected at each time point and the exact processing steps will depend on results identified on Aim1.2. Pending those results and based on the preliminary plan, samples will be centrifuged within 4-hour of collection=at 2,000 g for 30 minutes. Supernatant will be aliquoted for storage at minus 80° C., urine pellet resuspended in RNA preservation solution TCEP (150 μl) and also stored at minus 80° C. In parallel to urine collection, blood will also be collected on 10 cc purple-top (EDTA) tube of blood at 1, 3, 6, 9 and 12 months. EDTA tube will be then be processed by centrifugation at 3,200 g for 15 minutes at 4° C. Plasma will then be aliquoted in 0.8 cc cryotubes and frozen at −80° C. within 4 hours of sample collection.

### Clinical Information

Basic health information from corresponding patients will be extracted from medical records to record transplant characteristics, immunosuppression used, presence of donor-specific antibodies and complications such as rejection, infections or cancer. Collected information with be entered on RedCap as detailed above. Patients will be divided into stable patients without complications, patients with an episode of rejection, patients with BK replication, patients with worsening kidney function but no rejection or BK infection, and patients with other complications. Samples will be tested by CRISPR/Cas13 assay and dynamics of biomarkers will be determined in the first year after transplantation in different groups. Biomarkers will be assessed for (early) predictive value in case of rejection and BK-virus compared to stable patients or patients with other complications.

### Statistical Analyses

The objectives of this aim are to determine the relationships between single candidate biomarkers, or combinations thereof, obtained during the first 12 months after enrolment with a composite clinical endpoint of: acute cellular rejection (BANFF grade IA or above), acute antibody-mediated rejection, BK nephropathy, or a change in renal function with a decrease of 30% or more on the estimated glomerular filtration rate between 3 months post-enrollment and 12 months. The primary analysis will employ a mixed effects random slopes model that includes all measurements during study follow-up (at baseline and months 1, 2, 3, 4, 6, 9, and 12). The model will have a fixed effect term for group (graft injury vs. no injury), time, and time-by-group interaction, and a random intercept and time term. The predictor of primary interest is the time-by-group interaction, which measures the difference in the average slope between the two groups. An additional model will be run that includes fixed terms for any covariates that are unbalanced between the groups at baseline.

The random slopes model was chosen because it is robust to missing data that depends on prior observed assessments. In other words, it is valid whenever data are missing at random, meaning that the reason data are missing depends only on data that have been observed. It is also better suited than a repeated measures model if subjects' visits vary from the planned study visit date. It is expected that the pattern of chemokine changes should support a linear model. If linearity does not hold even, various transformations can be considered, or using nonlinear mixed effects models to account for non-linearity.

Around 75 patients receive a kidney transplant at the BWH each year and most patients are willing to participate in studies that do not involve a major intervention (>90%). Of these patients, approximately 15-20% will have an episode of rejection, and another 10-15% will develop BK infection. It is estimated that about 10% of the transplant population at the BWH will have a reduction of immunosuppression at any time post-transplant (currently 954 patients are followed post-transplant). Similar statistics are also seen in Brazil, where about 2,108 patients are followed post-transplant. Groups will be determined on presence or absence of complications (BK-virus, rejection, deterioration of kidney function, other complications, no complications). The availability of the surveillance biopsy at 6 months of recruitment and for-cause kidney biopsies will also allow assessment of the diagnostic power of these biomarkers.

### Potential Pitfalls and Alternative Approaches

Urinary expression analysis of samples is difficult to standardize (52), since the molecule levels depend on the overall levels of proteinuria and urinary concentration, which depend on fluid intake. Urine creatinine will be assessed simultaneously in order to allow measurement of chemokine/Cr ratio. Nonetheless, recent study indicated that results are significant even without any correction for urinary creatinine concentration (7). Based on the potential limitation of the stability of mRNA in the urine, plasma will be simultaneously collected from these patients, in which one could perform plasma mRNA analyses using the CRISPR/Cas13 assay in parallel to the urine analyses, to validate the findings and consider this alternative approach depending on the diagnostic and prognostic power of the biomarkers.

### Example 5. Concluding Remarks

This projects aims to develop a novel multiplexed point-of-care diagnostics for monitoring kidney transplant recipients that could be broadly applied worldwide. This is particularly relevant since there is a major need for accurate biomarkers to monitor kidney transplant patients as current gold standards are inefficient, invasive and costly. With the advancements of CRISPR technology in combination with the preliminary data confirming the feasibility to detect specific DNA and mRNA transplant biomarkers, this technology can be successfully applied to improve clinical care in transplantation. The low-cost and high-sensitivity of the assay will be particularly impactful in countries such as Brazil where traditional BK assays and other monitoring tools are not performed due to high cost. In regard to rejection markers, one can also envision a prospective trial emerging from this technology in which patients will be randomized to either standard of care or biomarker-guided management with the goal of earlier detecting graft injury and providing a timely intervention, improving long-term graft survival. Finally, the potential impact of this technology is beyond the transplant field, since this assay could be easily adapted for the monitoring of immune-mediated kidney disease in non-transplanted patients (e.g., lupus nephritis, ANCA vasculitis and membranoproliferative glomerulonephritis).

### Example 6. Material and methods for Example 7

Lateralflow reactions: 20 μl of the Sherlock reaction containing the lateral flow reporter-oligo at 1 μM were mixed with 80 μl of Hybridetect Assay buffer, followed by insertion of lateral flow-sticks (Milenia Hybridetectl, Twistox Limited, Maidenhead, UK) and incubation for 3 mins at room temperature according to the manufacturer's instructions before images were taken with a smartphone camera.

Sample preparation: Patient samples containing CMV or BKV were prepared as indicated either with the previously described HUDSON protocol (53) or the QIAamp MinElute Virus Spin Kit (Qiagen, Hilden, Germany) according to the manufacturer's instructions. For HUDSON processing, the samples were heated for 10 mins at 95° C. in presence of presence of 100 mM TCEP (FISHER SCIENTIFIC) and 1 mM EDTA (FISHER SCIENTIFIC). For CXCL9 mRNA detection 45 ml urine was centrifuged for 30 mins at 2000 g at 4° C., followed by washing of the pellet with PBS and resuspension in 200 μL RNAlater (Qiagen, Hilden, Germany). All samples were aliquoted and stored at −80° C. RNA was isolated using the RNeasy Micro Kit (Qiagen, Hilden, Germany) and ISA's kit.

Production of crRNAs and LwaCas13a: LwaCas13a was produced by Genscript (Piscataway, USA). crRNAs were synthesized using HiScribe™ T7 Quick High Yield RNA Synthesis Kit (NEB, Ipswich, USA) according to the manufacturer's instructions with T7 promoter containing annealed oligonucleotides. Reactions were incubated for 16 hours at 37° C., DNAse (NEB) digested and purified using the RNA Clean & Concentrator-25 kit. (ZymoResearch, Irvine, USA).

RPA primer and crRNA design: Genetically conserved regions in the BKV and CMV genome were identified using publicly accessible databases (Virus Pathogen Resource and NCBI). Alignments were performed using MAFFT (54) and visualized with Jalview (14). RPA primer design was done using NCBl's PRIMERBLAST tool with previously described settings4. CXCL9 RPA primers were designed to be in proximity to previously published qPCR primers (17). For each region to be amplified optimal primer pairs were identified by forward and reverse primer screens. Primer concentrations were optimized testing different forward and reverse primer concentrations in a dilution matrix. crRNAs, 28 nucleotides complementary to the target region, were designed as previously described (14, 55) and tested for their performance with each RPA primer pair.

qRT-PCR: RNA isolation, reverse transcription and quantitative PCR were performed as previously described (17). In-vitro transcribed RNA for CXCL9 and 18S rRNA served as standards.

RPA reactions: For RPA reactions the TwistAmp Liquid Basic kit (Twistox Limited, Maidenhead, UK) was used according to the manufacturer's instructions with the following modifications. Primer concentrations were 120 nM for the forward primer and 480 nM for the reverse primer. The total reaction volume was 20 μL with a final concentration of dNTPs at 7.2 mM (each) and MgOAc at 8 mM. RPA reactions were incubated at 37° C. for 50 mins.

For rt-RPA reactions forward and reverse primers were used at 480 nM each and MgOAc at 14 mM. 1 μL GoScript reverse transcriptase (Promega) was added to a 20 μL reaction containing DL-Dithiothreitol solution (OTT, Sigma-Aldrich) at a final concentration of 19 mM. The primer-RNA mix was pre-incubated at 65° C. for 10 mins and the rt-RPA reaction was performed at 42° C. for 60 mins.

Cas13 reactions: Detection of (rt)RPA amplified targets was performed as described previously (14, 55) with minor modifications. NEB buffer 2 (NEB, Ipswich, USA) served as cleavage buffer at a final concentration of 1×. 3 μL of RPA or rtRPA product were used in a 20 μL Cas13 reaction. Fluorescence (485 nm excitation, 520 nm emission) was measured on a plate reader (SpectraMax M5, Molecular Devices, San Jose, USA) every 5 mins for up to 3 h at 37° C.

One-pot reaction: One pot RPA-CRISPR reactions were performed with murine RNAse Inhibitor (NEB) at 1 U/L, Cas13 at 45 nM, crRNA at 22.SnM, RNAse Alert V2 (Thermofisher) at 125 nM, human background RNA (from 293T cells) at 1.25 ng/μL, T7 polymerase (Lucigen) at 0.6 μL/20 μL, dNTPs at 1.8 mM (each), rNTPs at 0.SmM (each), MgOAc at 18 mM and the buffers of the RPA TwistAmp Liquid Basic kit (2×, 10× and 20× buffers) at 1× final concentrations.

Diagnostic BKV and CMV quantitative PCR: Quantification of BKV and CMV viral load were performed at the CLIA certified diagnostic core facility at Brigham and Women's Hospital.

### Example 7. CRISPR-Based Diagnostics for Personalized Transplantation Medicine

Since the first organ transplant in 1954, significant improvement in short-term transplant outcomes has been achieved. However, long-term outcomes are stagnant with more than half of the transplanted organs being lost at 10 years post-transplant. Opportunistic infection and transplant organ rejection are leading causes of graft loss, requiring careful adjustment of drug dosages and life-long monitoring of transplant patients. However, until to date, diagnostics involve expensive laboratory equipment and intricate multi-step protocols leading to limited availability, high-costs and slow turn-over time. This ultimately delays diagnoses and increases the risk of irreversible injury, especially in resource-poor countries.

The clustered regularly interspaced short palindromic repeats (CRISPR) and CRISPR-associated (Cas) immune system has recently been adapted as a platform for the detection of nucleic acids (54, 14, 17, 55). These protocols enable rapid, cost-effective DNA and RNA diagnostics in a variety of sample types with excellent sensitivity and specificity, making them an ideal tool for point-of-care testing (POCT). However, most of the current studies mainly use synthetic standards, include few clinical specimens and lack direct comparison to the clinical gold-standard diagnostics.

Here, the CRISPR-Cas13 platform SHERLOCK (specific high-sensitivity enzymatic reporter unlocking) was applied and optimized for diagnosis of cytomegalovirus (CMV) and BK virus (BKV) infection, two common opportunistic viruses highly relevant for kidney transplant patients. Testing of more than 100 clinical specimens over a wide range of viral loads revealed a diagnostic performance that matches Clinical Laboratory Improvement Amendments (CLIA) standard quantitative PCR. The capability of SHERLOCK was further extended to the detection of human CXCL9 mRNA, a biomarker indicative of rejection in renal transplant patients (6-8, 10). It is anticipated that CRISPR-Cas13 will be broadly applicable for personalized medicine diagnostics, where repeated testing of biomarkers indicative for disease activity is a key to early and effective secondary prevention.

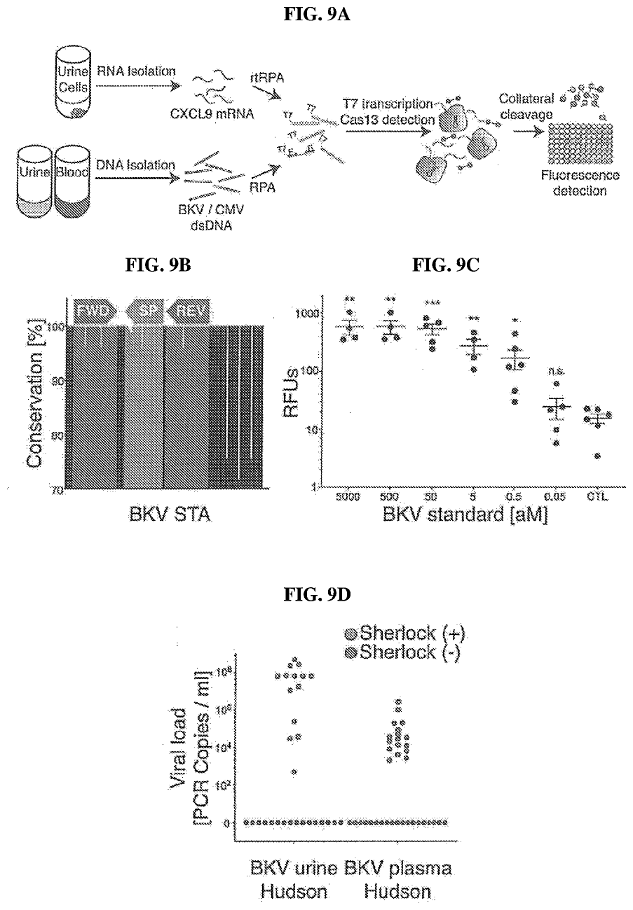

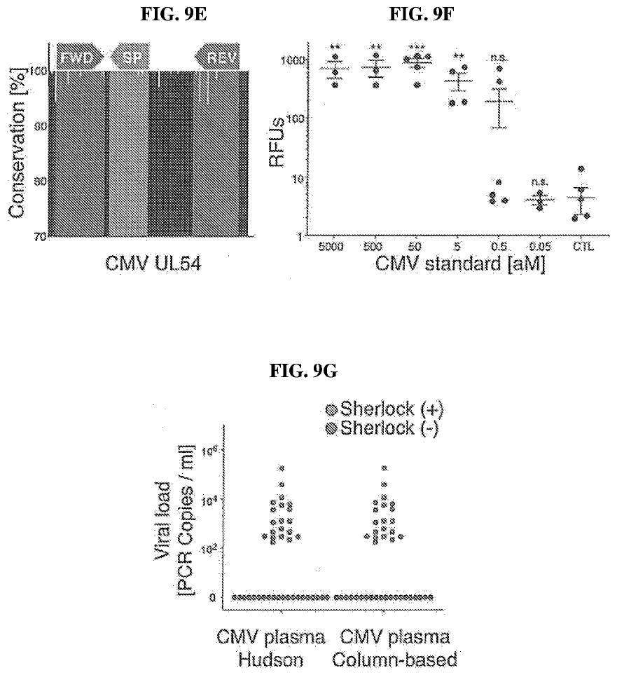

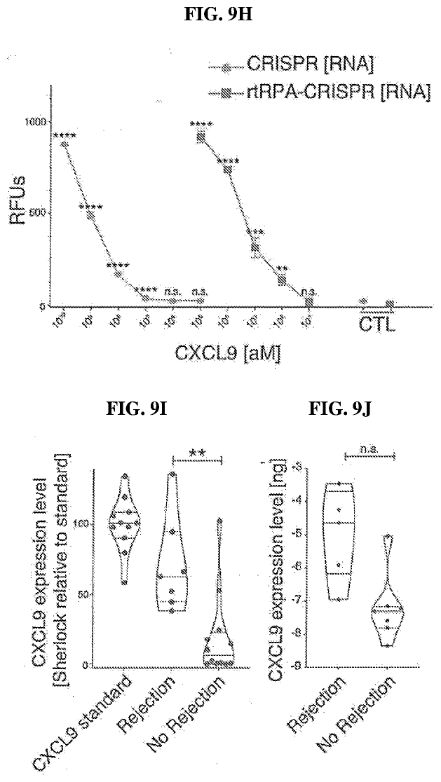

To test for BKV and CMV infection, DNA was isolated from blood and urine of infected patients and controls (FIG. 9A). Subsequently, a modified version of the SHERLOCK protocol was applied for target detection. In brief, conserved regions of BKV and CMV were amplified using isothermal recombinase polymerase amplification (RPA). Incorporation of the T7 promoter sequence into forward primers allowed for subsequent in-vitro RNA transcription using T7 polymerase. A guide RNA (crRNA) complimentary to 28 nucleotides (nts) of the RPA product was used to direct Cas13a from Leptotrichia wadei (LwaCas13a) to the target sequence. Detection of the target resulted in Cas13 activation and subsequent collateral cleavage of an oligonucleotide carrying a quenched fluorophore which fluorescence can be measured upon cleavage and correlates with the initial amount present in the patient sample.

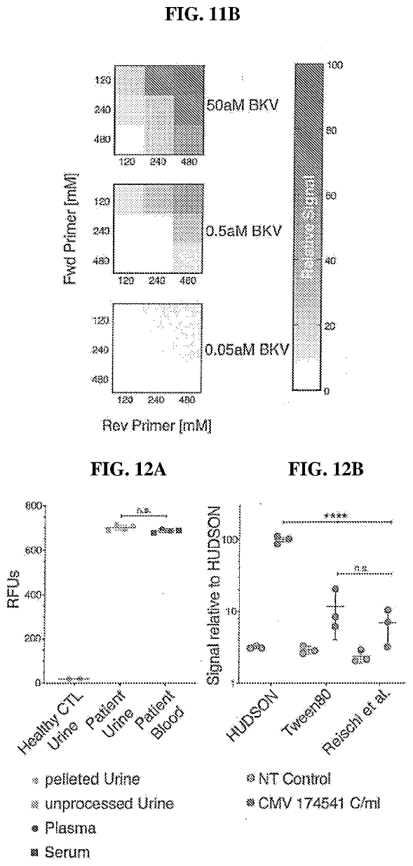

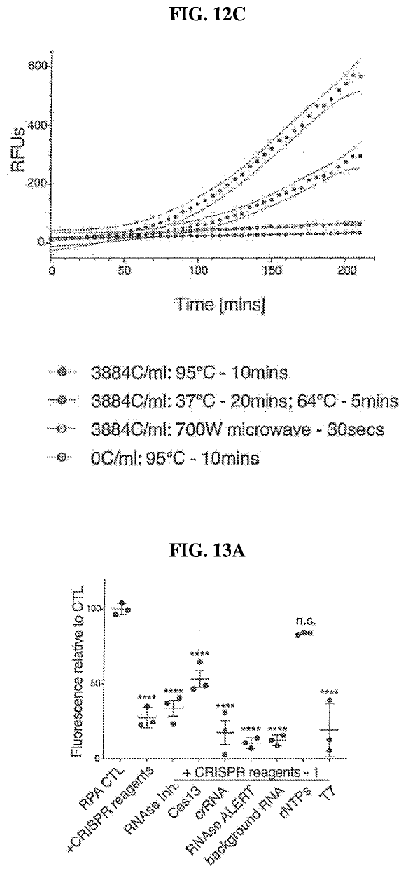

To identify conserved regions in the BKV genome, all different strains accessible from NCBI were aligned. Target regions with sequence homology of more than 95% among all strains were of highest interest (FIG. 9B). Next, 12 different primer pairs and 3 crRNAs were tested for detection of the BKV genes STA, VP2 and VP3 (FIG. 11A). A crRNA-primer pair specific for the small T antigen (STA) was identified which detected the ATCC quantitative synthetic BKV standard down to 0.5 aM (FIG. 9C). Importantly, systematic assessment of various forward and reverse primer concentrations (FIG. 11B) revealed a 120/480 nM forward/reverse RPA primer concentration to be most sensitive. Testing of urine and plasma samples from patients using this optimized Sherlock protocol correctly identified all specimen with 100% sensitivity and specificity as compared to the gold standard quantitative PCR (qPCR) which was performed in a Clinical Laboratory Improvement Amendments (CLIA) certified diagnostic lab (FIG. 9D, FIGS. 12A-12B). Importantly, this performance could be achieved using the rapid and simple HUDSON (heating unextracted diagnostic samples to obliterate nucleases) protocol, which involves heating of the sample for 10 mins at 95° C. in presence of TCEP and EDTA, circumventing the need of time-consuming column-based sample preparations (FIGS. 12C-12D).

Using a similar strategy, a conserved region in the CMV UL54 gene (FIG. 9E) was identified as a potential Sherlock target which allowed detection of the diagnostic CMV standard down to 5 aM (FIG. 9F). This allowed detection of all CMV+ plasma samples with a 100% sensitivity and specificity as compared to CLIA standard diagnostic qPCR (FIG. 9G). In contrast to the BKV results, this performance could only be achieved using a standard viral DNA isolation kit (CMV MinEiute) whereas the HUDSON protocol resulted in lower sensitivity for low copy number samples(<1500 C/ml).

Next, experiments were performed to test if SHERLOCK could be applied to detect mRNA biomarkers indicative of kidney graft rejection. CXCL9 mRNA was selected as a marker of rejection based on its validation in multicenter studies. Using a synthetic RNA standard, Cas13 only was sufficient to detect CXCL9 down to low pico-molar range similar to the previously reported sensitivity (FIG. 9H). Addition of a reverse transcriptase to the RPA reaction (rtRPA) followed by T7 transcription and Cas13 activation enabled CXCL9 detection in the atto-molar range (FIG. 9H). Applying the rtRPA CRISPR-Cas13 reaction to patient urine cells, higher CXCL9 mRNA levels were observed in samples from patients with biopsy-proven rejection compared to transplant patients with no rejection or stable graft function (FIG. 9I).

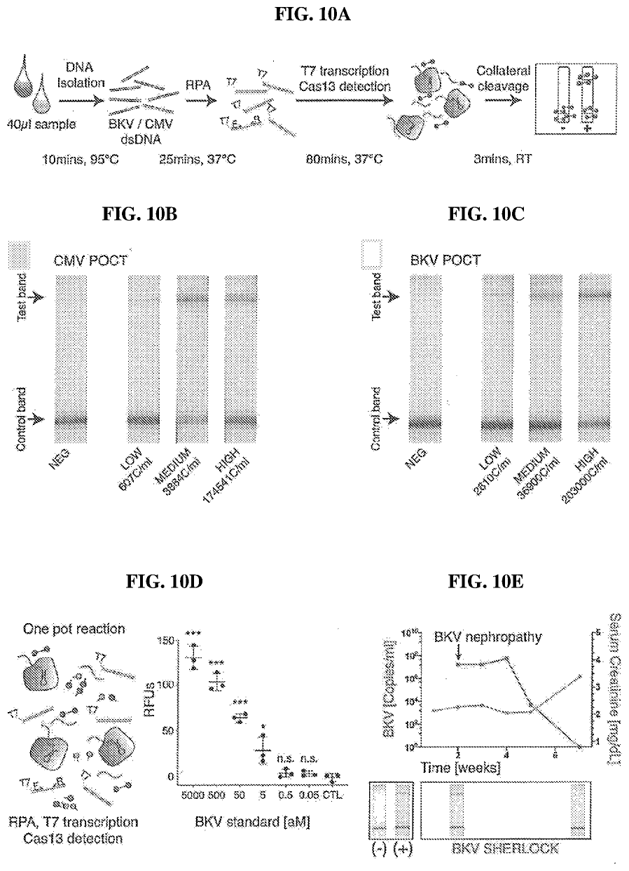

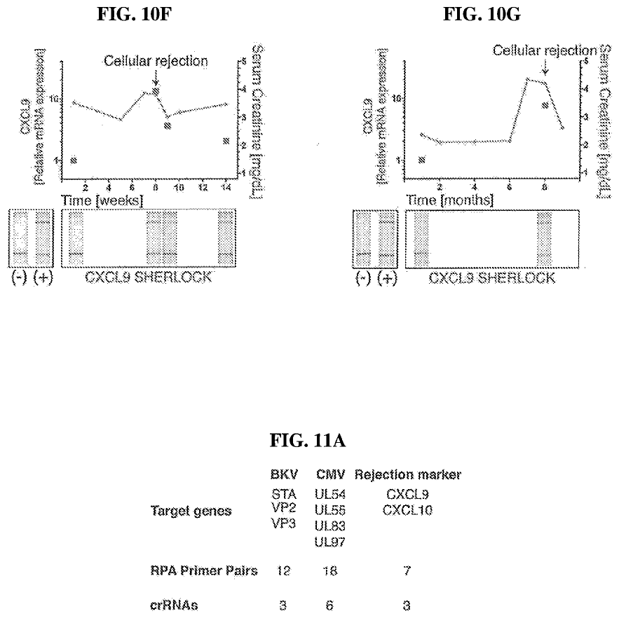

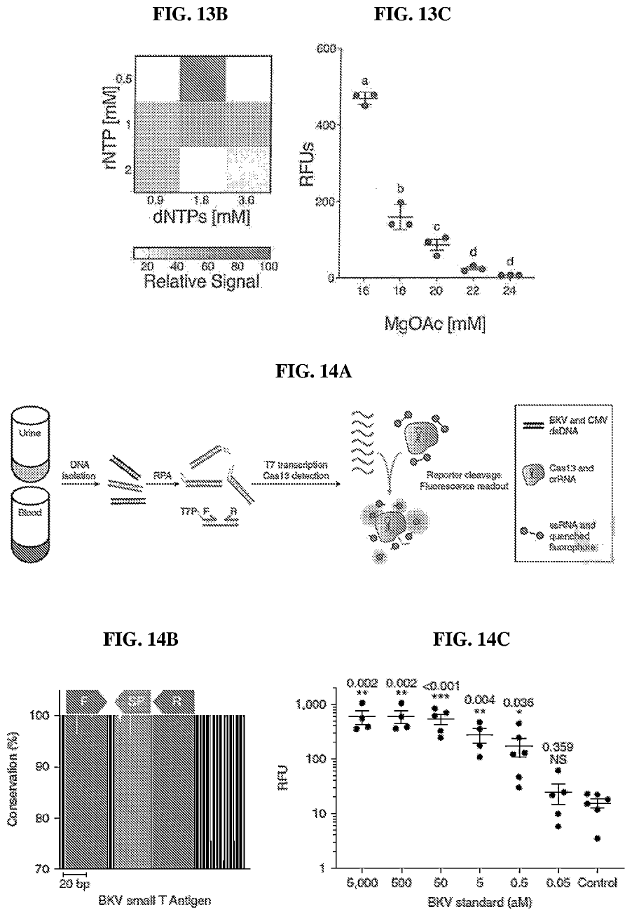

Point-of-care testing (POCT) holds great promise for transplantation medicine since fast and lowcost diagnostics could enable earlier treatment decisions and broader accessibility, thereby lowering the risk of long-term transplant irreversible injury. To optimize BKV and CMV detection for POCT, the rapid HUDSON DNA isolation protocol was combined with SHERLOCK-based target detection and commercially available lateral-flow dip sticks (FIG. 10A), enabling an easy-to-read visual output. The total turn-over time from isolation to sample detection by visual inspection was below 2 hours. Using this protocol, low copy numbers of CMV (FIG. 10B) and BKV (FIG. 10C) were detected in patient samples. In contrast to previously published experiments, a combination of RPA, T7 transcription and Cas13 in one reaction (“one-pot reaction”, FIG. 10D) needed optimization of reaction buffers and nucleotide ratios (FIGS. 13A-13D). However, using the optimized one-pot reaction, BKV detection was achieved down to the atto-molar range (FIG. 10D).

Finally, experiments were performed to apply the SHERLCOK-based POCT read-out of infection and rejection to monitor disease in three patients prospectively. SHERLOCK successfully identified BKV infection in a 58-year-old male kidney transplant patient who was admitted with graft dysfunction for a kidney biopsy. Management of BKV infection by intravenous immunoglobulin, solumedrol pulse and reduction of mycophenolate mofetil allowed the patient to successfully clear BK virus as confirmed by absence of viral copies in PCR and a negative SHERLOCK test result (FIG. 10E). Next, experiments were performed to test for CXCL9 mRNA in two patients undergoing rejection (FIGS. 10F-10G). In both patients, CXCL9 mRNA was not detected on lateral flow assays in urine pellets obtained at prior clinical visits. However, CXCL9 upregulation was observed during biopsy-proven acute cellular rejection by qPCR and successfully detected its expression using SHERLOCK. Since CXCL9 elevation in the urine can be detected weeks before elevation of creatinine due to rejection, urine CXCL9 monitoring with CRISPR/Cas13 represents a promising technique for earlier rejection detection. Furthermore, persistent elevation of CXCL9 after treatment is also an indicator of resistant rejection, serving as a marker of treatment response.

POCT holds great promise for the fast and cost-effective detection of disease, enabling early diagnosis and greater accessibility for patients in low-resource settings. Here, CRISPR-Cas13 diagnostics were applied to detect CMV and BKV infection with PCR-like sensitivity and specificity in human patient samples. The use of SHERLOCK was extended for the detection of the human mRNA biomarker CXCL9. Together, this enables a powerful platform for the cost-effective monitoring of patients at risk for opportunistic infection and may serve as a novel tool for risk stratification of rejection in transplantation medicine.

Although most steps could be optimized for a POCT setting, sample isolation for the detection of mRNA still required a column-based approach. Further optimization will reveal if mRNA biomarker can be robustly detected upon simplified isolation procedures.

These studies were mainly aimed for the qualitative detection of CMV, BKV and CXCL9 at clinically relevant concentrations. However, in many clinical situations, precise quantification of the viral load and changes in biomarker levels are useful. Future iterations of this protocol should, therefore, include quantitation strategies and may build on recent protocols demonstrating semiquantitative read-outs of CRISPR diagnostics. This would also strengthen the power of CRISPR-based diagnostics, since it could allow for the detection of subtle changes as a deviation of an individualized baseline. Finally, inclusion of more patient samples and prospective analysis will reveal the performance compared to current clinical practice.

### Example 8. Materials and Methods for Examples 9-13

Lateral-flow reactions: Twenty microlitres of the SHERLOCK reaction containing the lateral-flow reporter oligonucleotide at 1 μM (TABLE 3) were mixed with 80 μL of Hybridetect Assay buffer, followed by insertion of lateral-flow sticks (Milenia Hybridetectl, TwistDx) and incubation for 3 min at room temperature, according to the manufacturer's instructions, before images were taken.

Image analysis of lateral-flow reactions: The relative band intensities of each of the lateral-flow sticks were measured using ImageJ software (National Institutes of Health). The relative band intensity was calculated as the mean grey value of the test band/mean grey value of the control band. Images were first converted to 8 bit and inverted, before highlighting the band region and measuring its mean grey value.

Lateral-flow quantification app: The lateral-flow quantification algorithm was implemented using the opencv package in Python (v4.1.1). In brief, images uploaded to the app are automatically converted to greyscale and the colors are inverted. The resultant image is then subjected to a Gaussian blur to remove outlier pixels that may result in artefactual bright spots. A threshold is then applied to accentuate bright spots. Connected-component analysis is then used to isolate regions corresponding to the control and sample bands. These bands are then identified and quantified by calculating the mean intensity of each band. If the sample band cannot be identified due to weak intensity, the sample band's location is estimated by scanning for bright areas in the upper portion of the lateral-flow stick using the control band as a perspective scale. The ratio of the sample to control band is then calculated and displayed to the user. The Android app was developed with Android Studio v. 3.5.1 (Google) with Java 8 and Gradle v. 5.4.1 (Supplementary Video 1). To provide a clean user interface, the main screen was limited to three buttons: (i) upload new pictures, (ii) specify the target of the assay (that is, CMV, BKV or CXCL9), and (iii) initiate image analysis. The image-upload process requests read permissions to the phone's photo gallery. Image analysis allows two options, with the faster analysis scaling down the image to 50% lower resolution for more rapid results. The pixel array is passed to a Python back end through Chaquopy v. 6.3.0, a Python software development kit for Android.

Sample preparation: Patient samples containing CMV or BKV were prepared as indicated, either with the previously described HUDSON protocol (17) or the QIAamp MinElute Virus Spin Kit (Qiagen), according to the manufacturer's instructions. For HUDSON processing, the samples were heated for 10 min at 95° C. in the presence of 100 mM tris(2-carboxyethyl)phosphine (Fisher Scientific) and 1 mM EDTA (Fisher Scientific). For CXCL9 mRNA detection, 45 mL urine was centrifuged for 30 min at 2000 g at 4° C., followed by washing of the pellet with PBS and resuspension in 200 μl RNAlater (Qiagen). All samples were aliquoted and stored at −80° C. RNA was isolated using the RNeasy Micro Kit (Qiagen) and the PureLink RNA Mini Kit (Invitrogen), following the manufacturers' instructions.

Production of crRNAs and LwaCas13a: LwaCas13a was produced by Genscript (Piscataway). crRNAs were synthesized using the HiScribe T7 Quick High Yield RNA Synthesis Kit (NEB) according to the manufacturer's instructions, with the T7 promoter containing annealed oligonucleotides. Reactions were incubated for 16 h at 37° C., digested using DNAse (New England Biolabs) and purified using the RNA Clean & Concentrator-25 kit (ZymoResearch).

RPA primer and crRNA design: Genetically conserved regions in the BKV and CMV genome were identified using publicly accessible databases (Virus Pathogen Resource and NCBI). Alignments were performed using MAFFT (73) and visualized with Jalview (74). RPA primer design was done using the PRIMER-BLAST tool with previously described settings (15). CXCL9 RPA primers were designed to be in proximity to previously published qPCR primers (7). For each region to be amplified, optimal primer pairs were identified by forward and reverse primer screens. Primer concentrations were optimized by testing different forward and reverse primer concentrations in a dilution matrix. crRNAs, 28 nucleotides complementary to the target region, were designed as previously described (14, 15) and tested for their performance with each RPA-primer pair. The sequences, including spacer, direct repeat and T7 promoter, are shown in TABLE 3.

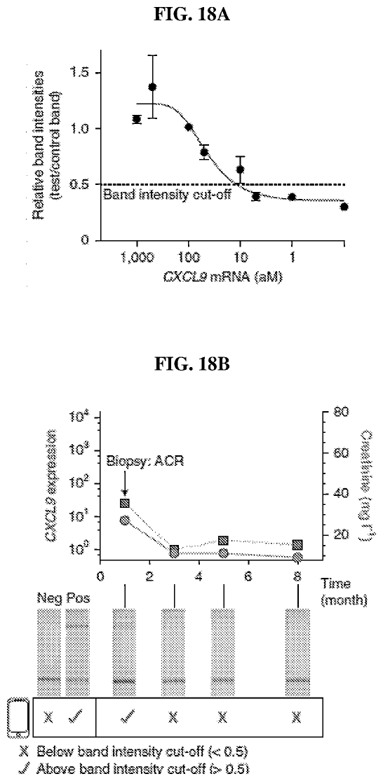

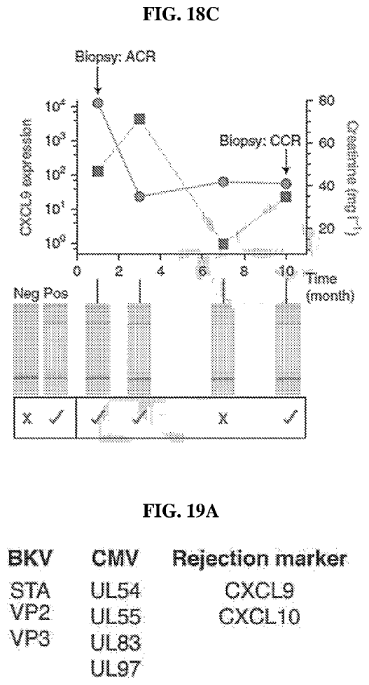

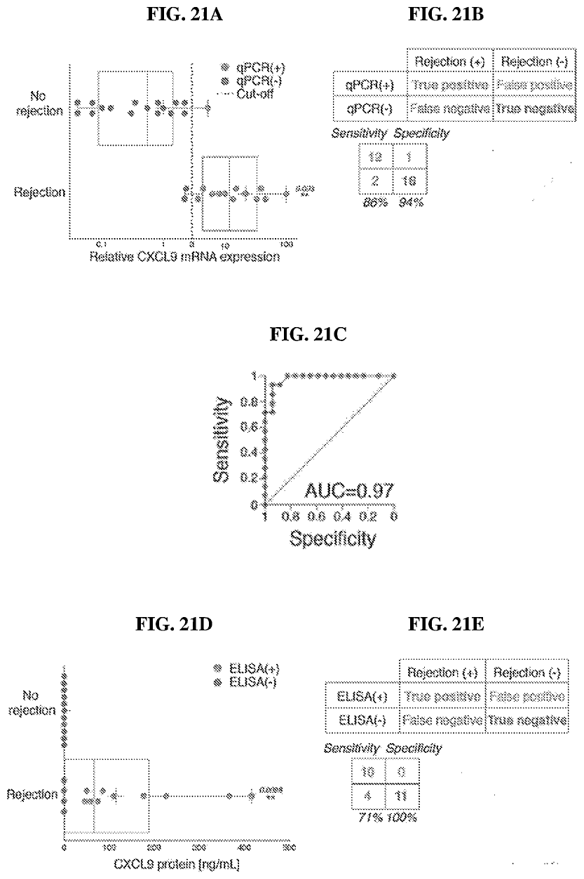

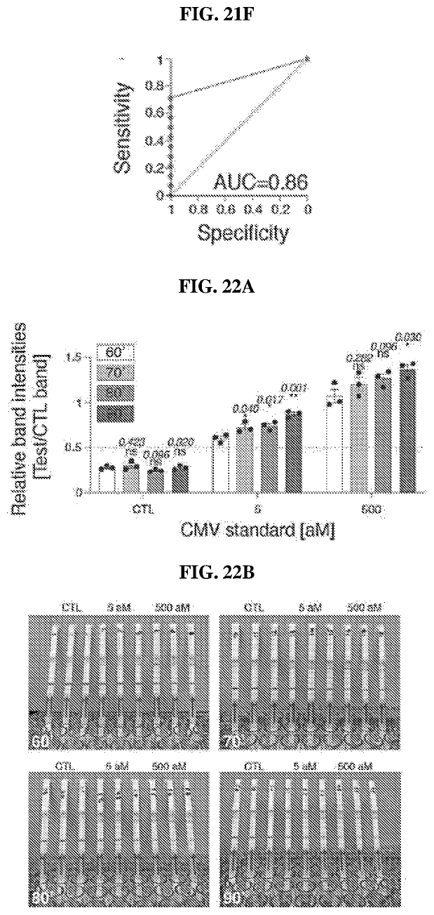

RT-qPCR: RNA isolation, reverse transcription and qPCR were performed as previously described18. In brief, RNA was reverse transcribed using the TaqMan Reverse Transcription kit (ThermoFisher) with random hexamers. The qPCR was performed using cDNA without pre-amplification. qPCR reactions were set up as previously described (7). All reactions were performed in duplicate, using the Applied Biosystems StepOne Plus real-time PCR system (ThermoFisher). In vitro transcribed RNA for CXCL9 served as a standard (sequences in TABLE 3). For expression analysis, the comparative CT method (75) was employed for quantification relative to 18 S RNA (FIG. 21A) or used absolute quantification on the basis of a CXCL9 standard curve (FIGS. 18B-18C). Expression levels are presented on a logarithmic scale relative to the control, whose average expression was set to 1.

TABLE 3
Nucleic acid sequences. Underlining indicates
the T7 promoter sequence.

| | | |
|---|---|---|
| SEQ | | |
| ID | | |
| NO: | Name | Sequence (5′-3′) |

RPA Primer

| | | |
|---|---|---|
| 1 | BKV_STA_fwd | GAAATTAATACGACTCACTATAGGCATT |
| | | GCAGAGTTTCTTCAGTTAGGTCTAAGCC |
| 2 | BKV_STA_rev | AATTTTTAAGAAAAGAGCCCTTGGTTTG |
| | | GATA |
| 3 | CMV_UL54_fwd | GAAATTAATACGACTCACTATAGGGCAC |
| | | CAGCCGAACGTGGTGATCCGCCGATCGA |
| | | TGAC |
| 4 | CMV_UL54_rev | CTATCAGCAACTGGACCATGGCCAGAAA |
| | | AATCG |
| 5 | CXCL9_fwd | GAAATTAATACGACTCACTATAGGTATC |
| | | CACCTACAATCCTTGAAAGACCTTAAAC |
| 6 | CXCL9_rev | TTAGACATGTTTGAACTCCATTCTTCAG |
| | | TGTA |

qPCR Primer and Probes

| | | |
|---|---|---|
| 7 | CXCL9_fwd | CTTTTCCTCTTGGGCATCATCT |
| 8 | CXCL9_rev | AGGAACAGCGACCCTTTCTCA |
| 9 | CXCL9 probe | FAM-TACTGGGGTTCCTTGCACTCCAAT |
| | | CAGA-TAMRA |
| 10 | 18S_fwd | GCCCGAAGCGTTTACTTTGA |
| 11 | 18S_rev | TCCATTATTCCTAGCTGCGGTATC |
| 12 | 18S_probe | FAM-AAAGCAGGCCCGAGCCGCC-TAMRA |

Oligos for T7 synthesis of crRNAs

| | | |
|---|---|---|
| 13 | T7_fwd | GAAATTAATACGACTCACTATAGG |
| 14 | BKV_STA_rev | CTGTGTGAAGCAGTCAATGCAGTAGCAA |
| | | GTTTTAGTCCCCTTCGTTTTTGGGGTAG |
| | | TCTAAATCCCTATAGTGAGTCGTATTAA |
| | | TTTC |
| 15 | CMV_UL54_rev | CGCGTCAGCGGATCCACACGGACCTCGT |
| | | GTTTTAGTCCCCTTCGTTTTTGGGGTAG |
| | | TCTAAATCCCTATAGTGAGTCGTATTAA |
| | | TTTC |
| 16 | CXCL9_rev | GCCCTTCCTGCGAGAAAATTGAAATCAT |
| | | GTTTTAGTCCCCTTCGTTTTTGGGGTAG |
| | | TCTAAATCCCTATAGTGAGTCGTATTAA |
| | | TTTC |

gRNAs

| | | |
|---|---|---|
| 17 | BKV | GATTTAGACTACCCCAAAAACGAAGGGG |
| | | ACTAAAACTTGCTACTGCATTGACTGCT |
| | | TCACACAG |
| 18 | CMV | GATTTAGACTACCCCAAAAACGAAGGGG |
| | | ACTAAAACACGAGGTCCGTGTGGATCCG |
| | | CTGACGCG |
| 19 | CXCL9 | GATTTAGACTACCCCAAAAACGAAGGGG |
| | | ACTAAAACATGATTTCAATTTTCTCGCA |
| | | GGAAGGGC |

gRNA Target (Spacer) Sequence

| | | |
|---|---|---|
| 20 | BKV | TTGCTACTGCATTGACTGCTTCACACAG |
| 21 | CMV | ACGAGGTCCGTGTGGATCCGCTGACGCG |
| 22 | CXCL9 | ATGATTTCAATTTTCTCGCAGGAAGGGC |

Synthetic Targets

| | | |
|---|---|---|
| 23 | CXCL9 | GAAATTAATACGACTCACTATAGGATGA |
| | | AGAAAAGTGGTGTTCTTTTCCTCTTGGG |
| | | CATCATCTTGCTGGTTCTGATTGGAGTG |
| | | CAAGGAACCCCAGTAGTGAGAAAGGGTC |
| | | GCTGTTCCTGCATCAGCACCAACCAAGG |
| | | GACTATCCACCTACAATCCTTGAAAGAC |
| | | CTTAAACAATTTGCCCCAAGCCCTTCCT |
| | | GCGAGAAAATTGAAATCATTGCTACACT |
| | | GAAGAATGGAGTTCAAACATGTCTAAAC |
| | | CCAGATTCAGCAGATGTGAAGGAACTGA |
| | | TTAAAAAGTGGGAGAAACAGGTCAGCCA |
| | | AAAGAAAAAGCAAAAGAATGGGAAAAAA |
| | | CATCAAAAAAAGAAAGTTCTGAAAGTTC |
| | | GAAAATCTCAACGTTCTCGTCAAAAGAA |
| | | GACTACATAA |

Cleavage Reporter

| | | |
|---|---|---|
| 24 | Lateral Flow | 6FAM-mArArUrGrGrCmAmArArUrGr |
| | | GrCmA-BIO |
| | Fluorescence | RNAse ALERT V2 (Thermo) |

RPA reactions: For RPA reactions, the TwistAmp Liquid Basic kit (TwistDx) was used according to the manufacturer's instructions, with the following modifications. Primer concentrations were 120 nM for the forward primer and 480 nM for the reverse primer. The total reaction volume was 20 μL, with a final concentration of dNTPs at 7.2 mM (each) and magnesium acetate at 8 mM. RPA reactions were incubated at 37° C. for 50 min. For RT-RPA reactions, forward and reverse primers were used at 480 nM each and magnesium acetate was used at 14 mM. One microlitre GoScript reverse transcriptase (Promega) was added to a 20 μL reaction containing dl-dithiothreitol solution (Sigma-Aldrich) at a final concentration of 19 mM. The primer-RNA mix was pre-incubated at 65° C. for 10 min and the RT-RPA reaction was performed at 42° C. for 60 min.

Cas13 reactions: Detection of RPA or RT-RPA-amplified targets was performed as described previously (14, 15, 17) with minor modifications. NEB buffer 2 (NEB) served as cleavage buffer at a final concentration of 1×. Three microlitres of RPA or RT-RPA product were used in a 20 μL Cas13 reaction. Fluorescence (485 nm excitation, 520 nm emission) was measured on a plate reader (SpectraMax M5, Molecular Devices) every 5 min for up to 3 h at 37° C.

One-pot reaction: One-pot RPA-CRISPR reactions were performed with murine RNAse Inhibitor (NEB) at 1 U μL⁻¹, Cas13 at 45 nM, crRNA at 22.5 nM, RNAse Alert V2 (Thermofisher) at 125 nM, human background RNA (from 293 T cells) at 1.25 ng μL⁻¹, T7 polymerase (Lucigen) at 0.6 μL per 20 μL, dNTPs at 1.8 mM (each), rNTPs at 0.5 mM (each), magnesium acetate at 16 mM and the buffers of the RPA TwistAmp Liquid Basic kit (2×, 10× and 20× buffers) at 1× final concentrations.

Diagnostic BKV and CMV quantitative PCR: De-identified patient samples were provided by the Crimson Core at Brigham and Women's Hospital (BWH). Quantification of BKV and CMV viral load were performed at the Clinical Laboratory Improvement Amendments-certified diagnostic core facility at BWH. In brief, BKV viral load samples were processed using the Luminex Aries instrument (Luminex) and a laboratory-developed protocol for a probe-free, two-primer, real-time PCR system. Following amplification, a thermal melt was performed. The system software allows for a quantitation template, developed using a standard curve calibrated against the first World Health Organization International Standard for BKV, to be applied to raw data for production of a quantitative value, reported in copies per ml (C mL⁻¹). CMV viral load samples were processed using the Roche Cobas AmpliPrep/Cobas TaqMan CMV Test on the Roche-docked Cobas AmpliPrep/TaqMan instrument. This is a real-time PCR system that automates specimen preparation, PCR amplification, target detection and quantitation. Results are reported in International Units per ml (IU mL⁻¹).

Patient populations: For the CMV and BKV studies, de-identified samples collected for clinical testing for CMV and BKV viremia at the BWH were provided by the Crimson Core at BWH. Clinical reported results for CMV and BK viremia were then compared to CRISPR-Cas13-diagnostics results. For the rejection and BKV nephropathy samples, patients were recruited prior to a kidney-transplant biopsy to investigate an elevation of creatinine at BWH. Prospective sample collection was also performed in few kidney-transplant recipients between January 2019 and June 2019. Samples started to be collected after one month of transplantation to avoid the effect of surgery and ischemic time. Samples were then collected according to clinical visits for 3-5 collections within the first year of transplant. The kidney-transplant cohort is representative of kidney-transplant recipients in this geographical location and at a tertiary academic hospital.

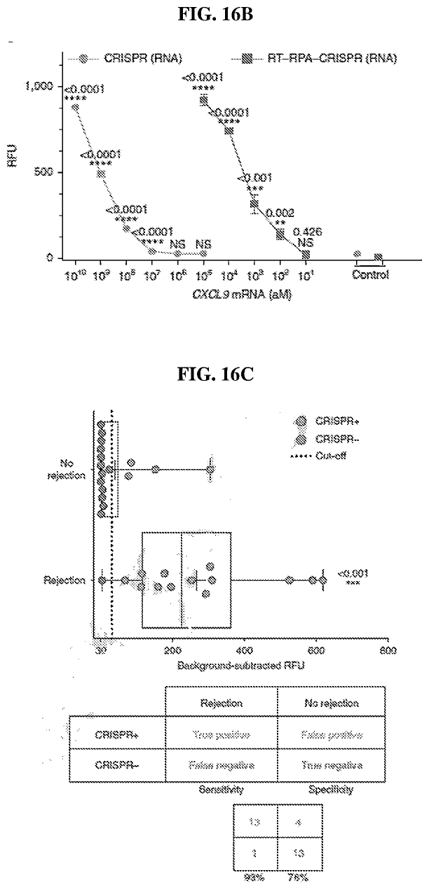

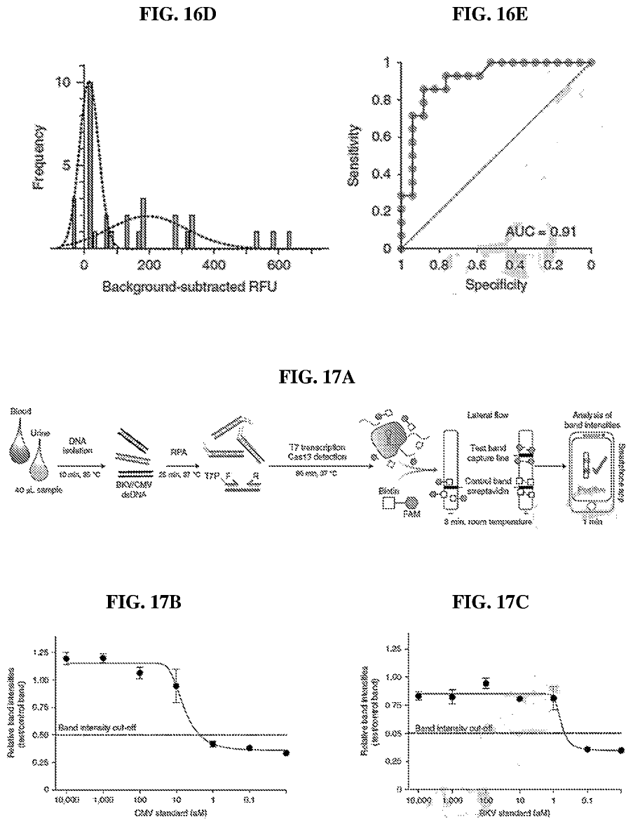

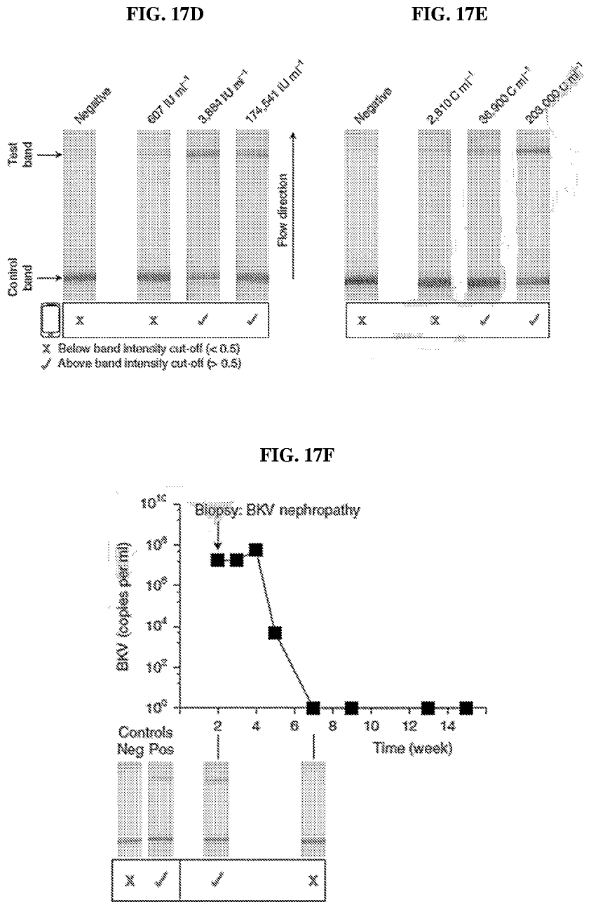

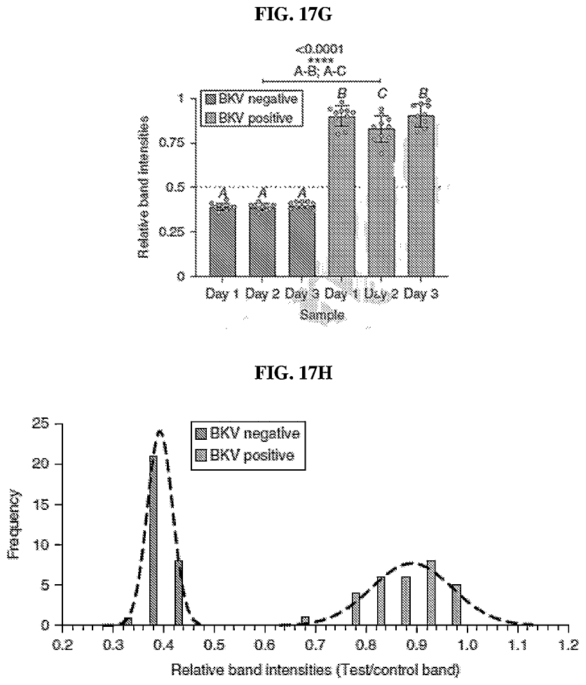

Statistics: CRISPR reactions were expressed as the mean of at least three independent reactions ±s.d. or s.e.m., as indicated in the figure legends. For statistical analysis, comparisons of patients with rejection and controls were conducted by unpaired two-tailed Student's t-test. Multiple group comparisons were conducted using one-way ANOVA and Tukey's multiple comparisons test. Comparisons of one control group to multiple others groups were performed using one-way ANOVA and Dunnett's multiple comparisons test. For histograms (FIG. 16D and FIG. 17H) GraphPad Software was used and the dashed lines were plotted using the following equation for a Gaussian distribution:

$$y = Amplitude \times {e}^{- 0.5 {( \frac{x - Mean}{s . d .} )}^{2}}$$

where x values represent the bin centre and y values represent the number of observations. Amplitude is the height of the centre of the distribution in y units. Mean is the x value at the centre of the distribution. The s.d. is a measure of the width of the distribution, in the same units as x. Statistical analyses were performed using GraphPadPrism 8.3.

Study design and participants: The study was approved by the Institutional Review Board at BWH (2017P000298), and the procedures followed were in accordance with institutional guidelines. In this observational study, a total of 31 kidney-transplant recipients were enrolled, and informed consent was obtained from all study participants (TABLE 4 and TABLE 5). Urine samples were collected from patients undergoing kidney biopsy for clinical indications. The cohort of samples was then selected on the basis of the presence of cellular rejection or no rejection on biopsy findings. For the prospective analyses, samples were provided by a cohort from Montefiore Medical Center, Bronx, N.Y. (Montefiore/Einstein Institutional Review Board (09-06-174). In brief, longitudinal samples were collected at the following time points: 0-3 months, 6-9 months and 9-12 months after transplant or when clinical biopsy was performed). Selection of patients was on the basis of availability of at least three samples collected either before or after a rejection event that was classified as rejection Banff IA or higher.

TABLE 4
Baseline and demographic characteristics of kidney transplanted patients.
Baseline characteristics are presented as mean ± SD. For noncategorical
variables, data were analyzed using Mann-Whitney test. For categorical
variables, data were analyzed using Fisher's exact test.

| | | | | |
|---|---|---|---|---|
| | All Subjects | Rejection | No Rejection | |
| Characteristics | (n = 31) | (n = 14) | (n = 17) | p-value |

Recipient Age (years)

| | | | | |
|---|---|---|---|---|
| Mean ± SD | 59 ± 13 | 61 ± 12 | 57 ± 14 | 0.589 |

Recipient Gender

| | | | | | | | |
|---|---|---|---|---|---|---|---|
| Female (n, %) | 17 | (54.8%) | 5 | (35.7%) | 12 | (70.6%) | 0.076 |
| Male (n, %) | 14 | (45.2%) | 9 | (64.3%) | 5 | (29.4%) | |

Recipient Race

| | | | | | | | |
|---|---|---|---|---|---|---|---|
| African American (n, %) | 10 | (32.2%) | 7 | (50.0%) | 3 | (17.6%) | 0.131 |
| Caucasian (n, %) | 14 | (45.1%) | 4 | (28.6%) | 10 | (58.8%) | |
| Other/Unknown | 7 | (22.6%) | 3 | (21.4%) | 4 | (23.6%) | |

Donor gender

| | | | | | | | |
|---|---|---|---|---|---|---|---|
| Female (n, %) | 20 | (64.5%) | 9 | (64.3%) | 11 | (64.7%) | 0.999 |
| Male (n, %) | 11 | (35.5%) | 5 | (35.7%) | 6 | (83.3%) | |

Donor Source

| | | | | | | | |
|---|---|---|---|---|---|---|---|
| Living (n, %) | 11 | (35.5%) | 4 | (28.6%) | 7 | (41.2%) | 0.707 |
| Deceased (n, %) | 20 | (64.5%) | 10 | (71.4%) | 10 | (58.8%) | |

Number of HLA mismatches

| | | | | |
|---|---|---|---|---|
| Mean ± SD | 4.4 ± 1.2 | 4.2 ± 0.9 | 4.6 ± 1.4 | 0.096 |

Induction Therapy

| | | | | | | | |
|---|---|---|---|---|---|---|---|
| Thymoglobulin (n, %) | 16 | (51.6%) | 5 | (35.7%) | 11 | (64.7%) | 0.268 |
| Basiliximab (n, %) | 14 | (45.2%) | 8 | (57.1%) | 6 | (35.3%) | |
| Alemtuzumab (n, %) | 1 | (3.2%) | 1 | (7.2%) | 0 | (0.0%) | |

Time since transplant (months)

| | | | | |
|---|---|---|---|---|
| Mean ± SD | 12 ± 12 | 14 ± 13 | 11 ± 12 | 0.256 |

Cause of kidney disease

| | | | | | | | |
|---|---|---|---|---|---|---|---|
| Diabetes | 7 | (22.6%) | 4 | (28.5%) | 3 | (17.6%) | 0.464 |
| Polycystic kidney disease | 5 | (16.2%) | 3 | (21.5%) | 2 | (11.8%) | |
| Polycystic kidney disease | 8 | (25.8%) | 3 | (21.5%) | 5 | (29.5%) | |
| Interstitial Nephritis | 3 | (9.6%) | 0 | (0.0%) | 3 | (17.6%) | |
| Other/Unknown | 8 | (25.8%) | 4 | (28.5%) | 4 | (23.5%) | |

TABLE 5
Diagnosis at the time of biopsy from rejection patients'
cohort. Data is expressed as mean ± SD. Banff score abbreviations:
glomerulitis (g), interstitial inflammation (i), tubulitis
(t), intimal arteritis (v) peritubular capillaritis (ptc),
interstitial fibrosis (ci), tubular atrophy (ct), vascular
fibrous intimal thickening (cv), glomerular basement membrane
double contours (cg), arteriolar hyalinosis (ah).

| Characteristics | N = 14 |
|---|---|
| Creatinine (mg/dL) | 3.24 ± 1.63 |
| eGFR (ml/min/1.73 m²) | 23.21 ± 9.27 |
| Rejection Type (n, %) | |
| Borderline | 1 (7.2%) |
| IA/IB | 8 (57.1%) |
| IIA/IIB | 4 (28.5%) |
| III | 1 (7.2%) |
| Banff g score | 1.5 ± 1.4 |
| Banff i score | 2.4 ± 0.8 |
| Banff t score | 2.1 ± 0.8 |
| Banff v score | 0.6 ± 0.9 |
| Banff ptc score | 1.5 ± 1.3 |
| Banff ci score | 1.2 ± 0.7 |
| Banff ct score | 1.2 ± 1.0 |
| Banff cv score | 1.2 ± 1.0 |
| Banff cg score | 0.6 ± 0.9 |
| Banff ah score | 0.6 ± 0.9 |
| C4d score | 0.6 ± 1.2 |

Ethics: All studies have complied with all relevant ethical regulations. The patient samples used in this study were obtained from the clinical study Biomarkers in Kidney Transplantation, which was approved by Partners Human Research Committee (2017P000298/PHS). Written informed consent has been obtained from all participants.

### Example 9. Optimization of the CRISPR-Cas13 SHERLOCK Technology for the Detection of BKV and CMV Virus from Patient Samples

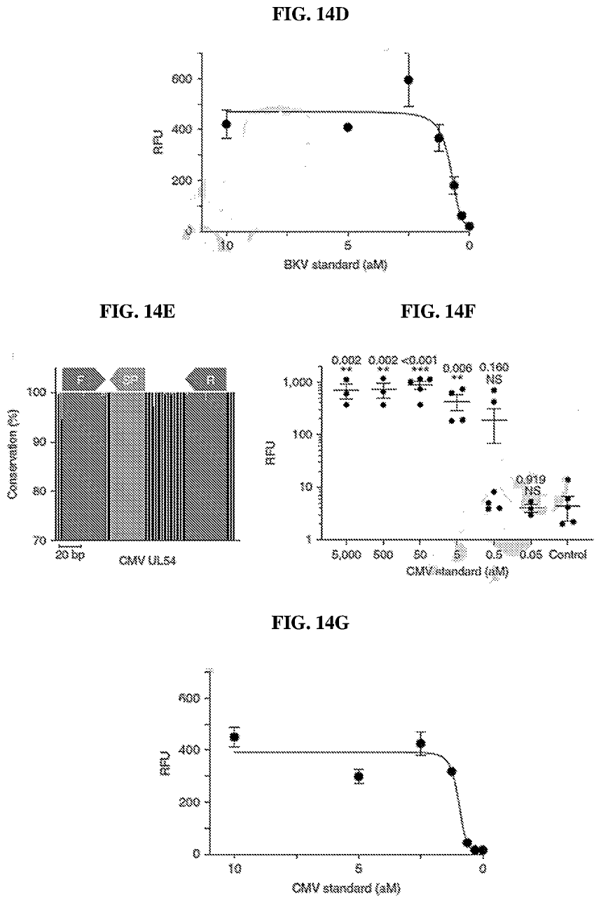

To test for active BKV and CMV infection, DNA was isolated from blood and urine of both infected patients and uninfected control patients (FIG. 14A). Subsequently, a modified version of the SHERLOCK protocol was applied for BKV and CMV detection. In brief, conserved regions of BKV and CMV were amplified using isothermal recombinase polymerase amplification (RPA). Incorporation of the T7 promoter sequence into forward primers enabled subsequent in-vitro RNA transcription using T7 polymerase. A CRISPR guide RNA (crRNA) complementary to 28 nucleotides of the RPA product was used to direct Cas13 from Leptotrichia wadei (LwaCas13a) to the target sequence. Detection of the target resulted in Cas13 activation and subsequent collateral cleavage of an oligonucleotide carrying a quenched fluorophore that exhibits fluorescence when cleaved, correlating with the initial concentration of the target in the patient sample (14).

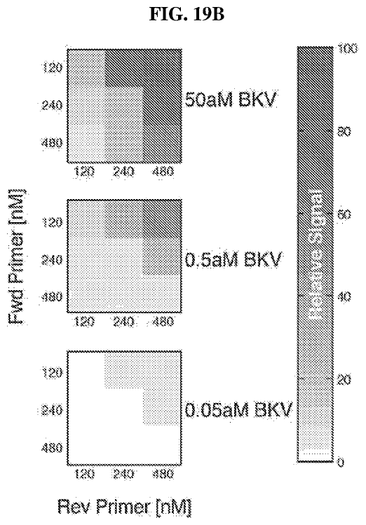

To identify conserved regions in the BKV genome, all strains accessible from the National Center for Biotechnology Information (NCBI) were aligned. Target regions with sequence homology of more than 95% among all strains were of interest (FIG. 14B). Next, 12 different primer pairs and 3 crRNAs were tested for their ability to detect the BKV genes STA, VP2 and VP3 (FIG. 19A). A crRNA-primer pair specific for the small T antigen (STA) was identified, which allowed detection of the American Type Culture Collection (ATCC) quantitative synthetic BKV standard (Dunlop strain) down to the low attomolar range (0.3 aM), representing single-molecule detection in the assay volumes used (FIGS. 14C-14D). Notably, systematic assessment of various forward and reverse primer concentrations (FIG. 19B) revealed that forward and reverse RPA primer concentrations of 120 nM and 480 nM, respectively, resulted in the highest sensitivity. Using a similar strategy, a conserved region in the CMV UL54 gene (FIG. 14E) was identified as a potential SHERLOCK target that enabled detection of the ATCC diagnostic CMV standard (strain AD-169) down to the low attomolar range (0.6 aM) (FIGS. 14G-14G).

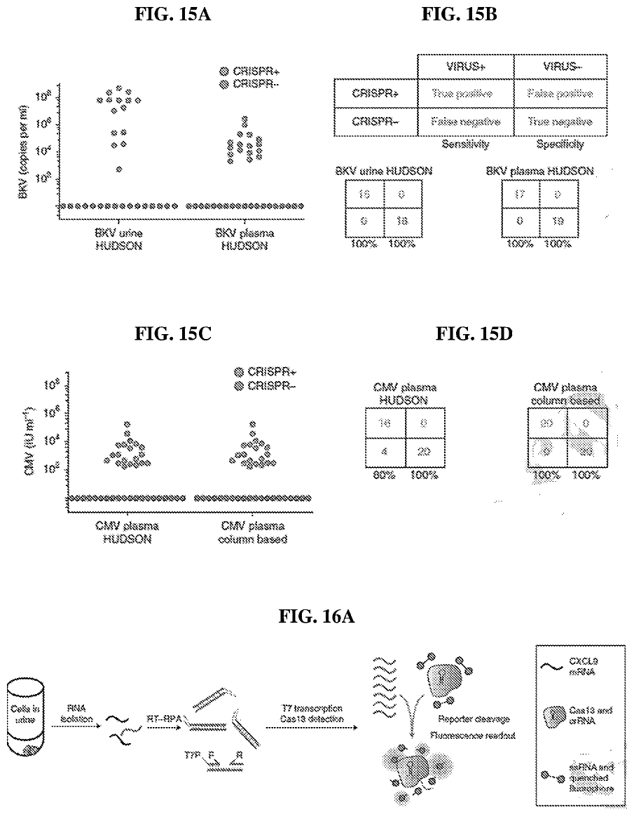

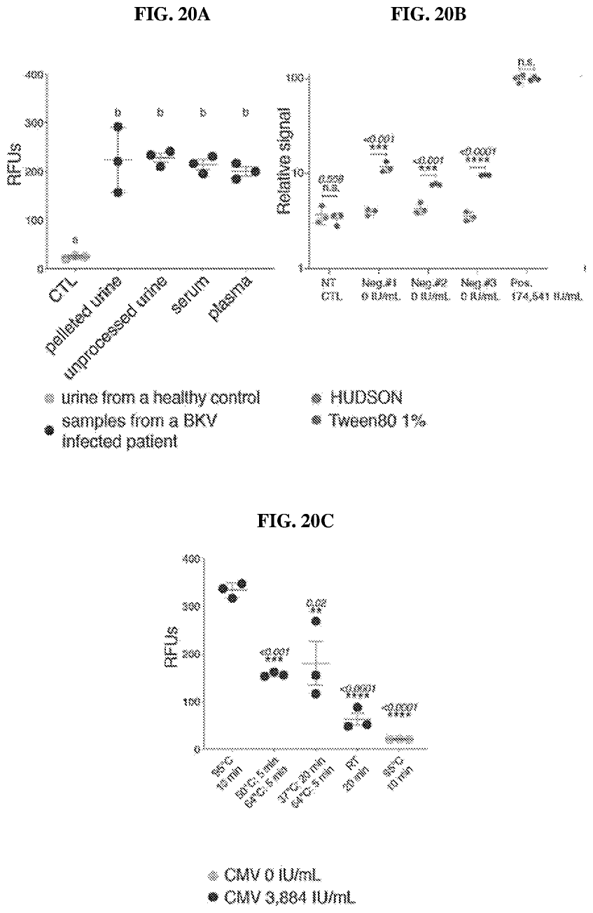

Next, studies were performed to test whether the diagnostic performance of the SHERLOCK assay would be sufficient to detect BKV and CMV virus in urine and plasma samples from patients. Testing of 31 urine and 36 plasma samples showed that the optimized SHERLOCK protocol correctly identified all BKV specimens with 100% sensitivity and specificity (FIGS. 15A-15B and FIG. 20A). Of note, this performance could be achieved using the rapid and simple heating unextracted diagnostic samples to obliterate nucleases (HUDSON) protocol (17), which involves heating of the sample for 10 min at 95° C. in the presence of tris(2-carboxyethyl)phosphine and EDTA, circumventing the need for time-consuming, column-based sample preparations (FIGS. 20B-20C).

Similarly, the CRISPR assay enabled the detection of CMV-positive plasma samples with high sensitivity and specificity (FIGS. 15C-15D and FIGS. 20B-20C). In contrast to results with BKV, this performance could only be achieved using a commercial column-based viral DNA isolation kit; the HUDSON protocol resulted in lower sensitivity for low-copy-number samples (<1500 IU ml⁻¹). This difference in sensitivity is probably due to a sample concentration step included in the column-based kit.

### Example 10. CRISPR-Based Detection of CXCL9 mRNA as a Biomarker of Kidney Graft Rejection

Next, experiments were performed to test whether SHERLOCK could be applied to detect mRNA biomarkers indicative of kidney graft rejection. CXCL9 mRNA was selected as a marker of rejection on the basis of its validation in multicentre studies (7, 10, 28).

For detection of CXCL9 mRNA, RNA was isolated from pelleted urine cells (FIG. 16A). For amplification, reverse transcriptase was added into the RPA reaction (RT-RPA). Using a synthetic RNA standard, Cas13 alone was sufficient to detect CXCL9 in the low picomolar range, similar to the previously reported sensitivity (FIG. 16B). Addition of an RT-RPA reaction followed by T7 transcription and Cas13 activation enabled CXCL9 detection in the attomolar range (FIG. 16B).

Experiments were next performed to test whether this sensitivity was sufficient to discriminate patients undergoing kidney rejection (n=14) from a control group (n=17) (TABLE 4). Rejection status was determined by gold-standard kidney biopsy (TABLE 5).

The results showed higher CXCL9 mRNA levels in samples from patients with biopsy-proven rejection compared with transplant recipients with no rejection or stable graft function, which enabled the detection of kidney rejection with a sensitivity of 93% (FIGS. 16C-16D). The area under the receiver-operating-characteristic (ROC) curve was 0.91 (FIG. 16E).

CXCL9 mRNA upregulation was confirmed in rejection samples with the quantitative PCR (qPCR) gold-standard assay (7), observing higher diagnostic accuracy compared with the CRISPR-based assay (FIGS. 21A-21C). Detection of CXCL9 protein with an enzyme-linked immunosorbent assay (ELISA) showed lower sensitivity but higher specificity (FIGS. 21D-21F) compared with CRISPR-based mRNA detection.

### Example 11. Rapid DNA Isolation, CRISPR Diagnostics and Smartphone-Based Lateral-Flow Evaluation Enable POC-Ready Detection of BKV and CMV Infection

POC testing holds great promise for transplantation medicine, since fast and low-cost diagnostics could enable earlier treatment decisions and broader accessibility, thereby lowering the risk of irreversible transplant injury. To optimize BKV and CMV detection for POC testing, the rapid HUDSON DNA-isolation protocol was combined with SHERLOCK-based target detection and commercially available lateral-flow dipsticks (FIG. 17A). This method enabled an easy-to-read visual output that indicated a positive or negative test result. Since background noise can result in a faint test band on the lateral-flow strip, a smartphone-based software application was developed that allowed quantification of band intensities. Here, the software calculates the ratio of test to control band intensities using images taken with a smartphone camera, enabling simple and rapid discrimination between negative and positive test results. The total turnaround time from isolation to sample detection was below 2 h.

Next, experiments were performed to test the lateral-flow readout for the detection of the CMV and BKV synthetic standard (FIGS. 17B-17C). Similar to the fluorescence-based readout, one could detect both targets down to the attomolar range. The relative band-intensity cut-off discriminating a positive from a negative test result was set to 0.5, which corresponded to an interpolated concentration of 2.3 aM for the CMV standard and 0.5 aM for the BKV standard.

Using this protocol, detection of CMV (FIG. 17D) and BKV (FIG. 17E) at different concentrations in patient samples was possible. Although faint test bands were observable at very low concentrations, they were below the band-intensity cut-off and thus classified as negative. Further, lateral-flow-based CRISPR diagnostics successfully identified BKV infection in a 58-yr-old male kidney-transplant recipient who was admitted for graft dysfunction. A kidney biopsy demonstrated BKV nephropathy and qPCR confirmed high viral BKV titres in the blood.

After treatment, one could not detect BKV using CRISPR-Cas13; this result was validated by the absence of viral DNA in qPCR (FIG. 17F).

To assess lateral-flow signal variability over time, the same ten BKV-positive or -negative patient samples were tested on three different days (FIGS. 17G-17H). All BKV-negative samples were consistently below the band-intensity cut-off on all three days, whereas all BKV positive samples were above the cut-off. This suggested a low variability of background noise and band intensities.

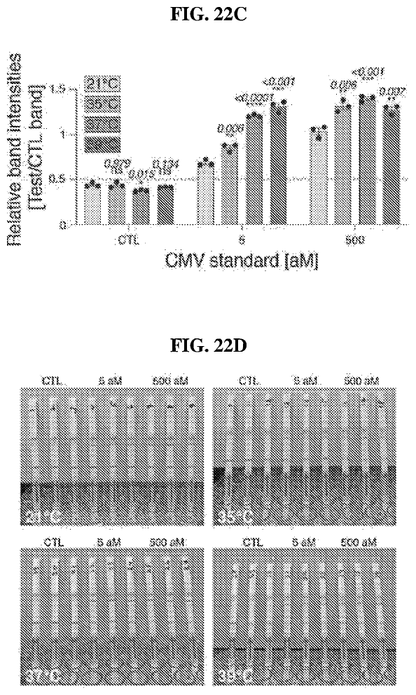

Experiments were also performed to assess the influence of incubation time and temperature of the CMV synthetic standard. For the negative control, the relative band intensity stayed below the 0.5 cut-off regardless of the incubation time, in line with the previous results (FIGS. 22A-22B). When detecting 5 or 500 aM of CMV synthetic DNA, a time-dependent increase of band intensities was observed. Notably, band-intensity values above the cut-off only after 60 min were observed, indicating that the assay incubation time could be further reduced. For different reaction temperatures ranging between 21° C. and 39° C., band-intensity ratios below 0.5 for the negative control were agains observed (FIGS. 22C-22D). While room temperature (21° C.) was sufficient to detect 5 or 500 aM of CMV synthetic standard, higher temperatures correlated with higher band intensities. These results indicate that reaction time and temperature are important variables if a quantitative lateral-flow readout is the goal. By contrast, highly consistent background noise irrespective of daily variation, incubation time and temperature enable a robust qualitative assay.

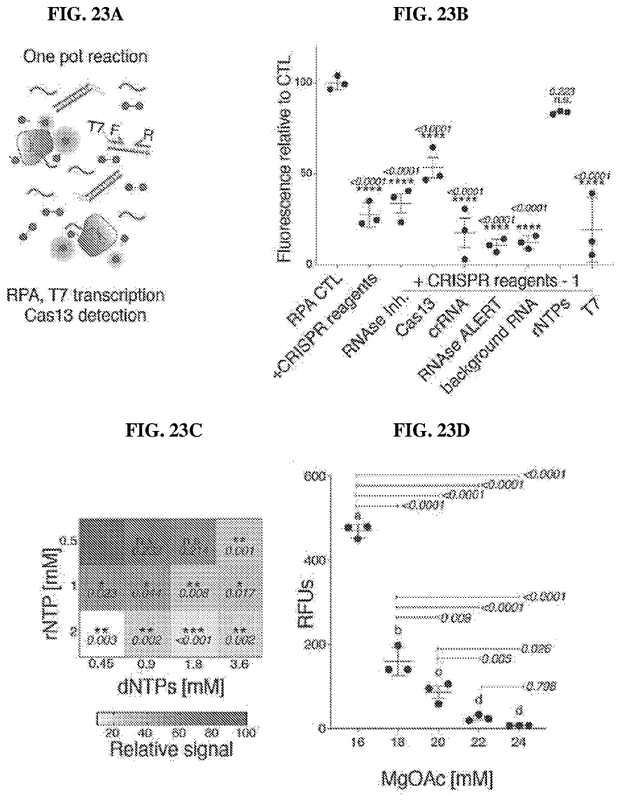

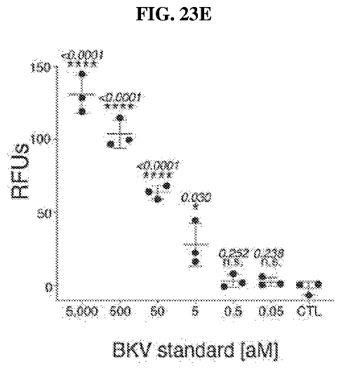

The combination of RPA, T7 transcription and Cas13 in one reaction (‘one-pot reaction’; FIG. 23A) was further optimized by testing different reaction buffers and nucleotide ratios (FIGS. 23B-23D). Using this optimized one-pot reaction, BKV detection in the attomolar range was achieved (FIG. 23E).

### Example 12. Detection of CXCL9 mRNA Levels with Lateral Flow Enables Monitoring of Kidney Rejection and Treatment Response

Next, the lateral-flow-based assay was applied for the detection of CXCL9 mRNA indicative of acute cellular kidney rejection. Similar to the detection of viral DNA, lateral flow enabled robust detection of CXCL9 synthetic RNA (FIG. 18A) down to the attomolar range. Using nonlinear regression analysis, a concentration of 12 aM corresponded to the 0.5 band-intensity cut-off was determined.

To investigate the power of the CRISPR-based readout for rejection monitoring, two patients experiencing allograft cellular rejection as confirmed by biopsy were selected, from whom we had at least three prospective samples after the rejection event (FIGS. 18B-18C). Patient 1 (FIG. 18B) developed an acute cellular rejection (Banff IIA) and showed a good response to treatment with thymoglobulin and pulse methylprednisolone, achieving full clinical recovery. This was reflected by a strong downregulation of CXCL9 mRNA levels as observed by qPCR and return to their baseline serum creatinine (9 mg l⁻¹). CRISPR-based testing detected CXCL9 mRNA during rejection only, and the patient was CXCL9 negative after treatment completion.

By contrast, Patient 2 (FIG. 18C) had an episode of acute cellular rejection (Banff IIA) with partial improvement of creatinine after treatment (Serum creatinine 35 mg l⁻¹, down from 79 mg l⁻¹). Levels of CXCL9 mRNA in the urine was reduced initially, but increased again 7 months after treatment, and repeat biopsy revealed chronic active cellular rejection. Overall, monitoring of urine CXCL9 mRNA levels may be a useful tool to assess response to rejection treatment, though further validation in a larger trial is needed.

### Example 13. Discussion

Fast and cost-effective POC testing should enable early diagnosis and greater accessibility for patients in low-resource settings, including the opportunity for self-monitoring. Here, CRISPR-Cas13 diagnostics were applied to detect infection with CMV and BKV in samples from recipients of kidney transplants. The use of SHERLOCK was extended for the detection of CXCL9 mRNA, a biomarker of acute cellular rejection of kidney transplants. Together, these developments may enable the cost-effective (TABLE 6) monitoring of patients at risk of opportunistic infection and serve as a tool for earlier detection of rejection and monitoring after organ transplantation.

TABLE 6
Comparison of CRISPR diagnostics with ELISA, biolayer interferometry
and qPCR for the detection of CXCL9, BKV and CMV.

| | | Biolayer | | CRISPR | |
|---|---|---|---|---|---|
| | ELISA | interferometry | | diagnostics | qPCR |
| Reference | RND systems | Gandolfini | | this paper | Altona |
| | (DCX900) | et al., | | | diagnostics, |
| | Hricik et al., | 2017 | | | RealStar Kits |
| | 2013 | | | | 021003 (CMV); |
| | | | | | 031003 (BKV) |
| Analyte | CXCL9 protein | CXCL9 protein | | CXCL9 mRNA; | BKV & CMV |
| | | | | BKV& | DNA |
| | | | | CMV DNA | |
| Speed | | | | | |
| Assay length | 4 h 30 min | 60 | min | 108 min | 85 min |
| Hands-on time | 1 h 20 min | 10 | min | 10 min | 10 min |
| Costs | | | | | |
| Equipment | >$5,000 USD | >$100,000 USD | | >$5,000 USD | >$5,000 USD |
| Reagents (per test) | $5.4 USD | (OctetRED96) | | (fluorescence) | $20 USD |
| | | $5.4 USD | | $2.3 USD per test | |
| | | | | (lateral flow) | |
| | | | | $1 USD | |
| Sensitivity | 11.3 pg/mL | 35 | pg/mL | low attomolar | low attomolar |
| | | | | range | range |

| | | | | |
|---|---|---|---|---|
| POCT | | | | |
| Compatibility | | | | |
| Isothermal | Yes | Yes | Yes | No |
| incubation | No | No | Yes (lateral flow) | No |
| Minimal equipment | No | No | Yes (lateral flow) | No |
| Visual output | | | | |

BKV and CMV are among the most common opportunistic infections after solid-organ transplantation, and are associated with significant morbidity (67). However, clinical presentation is variable in transplanted patients and BKV infection frequently presents without clinical symptoms except an increase in creatinine, which indicates already established BKV nephropathy. Blood testing for BKV and CMV viral load is recommended but is not uniformly performed in all centers due to cost limitations, particularly in developing countries. Here, the high-sensitivity, low-cost POC assay could allow for more frequent testing.

Rejection is the leading cause of chronic allograft loss. However, rejection is usually detected late since serum creatinine is a delayed marker of allograft injury. Furthermore, the diagnosis of acute rejection currently requires a renal biopsy—an invasive process that is limited by sampling error and assessment variability (4). In order to detect graft injury earlier, some centers perform surveillance kidney biopsies at prespecified time points after transplant (18). However, these procedures are associated with major risks for patients, such as bleeding, and significant costs (about US$3,500 per biopsy, which includes the procedure and the pathological analyses of the kidney specimen). Therefore, a sensitive and non-invasive assay such as CRISPR-Cas13-based CXCL9 mRNA testing could enable more frequent testing and thereby achieve earlier detection of graft rejection, allowing timely diagnosis and treatment.

Currently, screening for donor-specific human leukocyte antigen antibodies (DSA) is performed in patients with concern for antibody-mediated rejection. All patients included in this study tested negative for DSA. Screening prospectively for DSA in all patients is not uniformly performed, in part due to lack of specificity of DSA to antibody-mediated rejection.

Besides DSA screening, two blood tests that detect the fraction of donor-derived cell-free DNA have become clinically available in kidney transplantation to monitor for rejection (37, 68). While these assays have shown promising results, they still require a visit to the clinic to draw blood and shipping of the material to outside laboratories for processing and analysis, and they have a high price tag of US$2,821 per test (sec.gov/Archives/edgar/data/1217234/000156459018006584/cdna-10k_20171231.htm). This high price limits the frequency of testing and also prevents the use of this test in resource-limited settings. The advantages of the rejection assay are its low cost, its high sensitivity and its use of urine compared to blood. Since elevation of CXCL9 mRNA in the urine can be detected weeks before elevation of creatinine due to rejection26, urine CXCL9 mRNA monitoring may represent a promising technique for earlier rejection detection as well as post-treatment monitoring. Lastly, the development of a smartphone application to enable simple and fast interpretation of the lateral-flow assay allows for sharing of results directly with the provider, leading to a convenient way of monitoring patients between clinical appointments.

This test was mainly aimed at qualitative detection of CMV, BKV and CXCL9 mRNA at clinically relevant concentrations. However, in many clinical situations, precise quantification of the viral load and changes in biomarker levels are useful. Future iterations of this protocol should, therefore, include quantification strategies and may build on recent protocols demonstrating semi-quantitative readouts of CRISPR diagnostics (15). This would also strengthen the power of CRISPR-based diagnostics, since it could enable the detection of subtle changes as a deviation from an individualized baseline. Moreover, although most steps could be optimized for POC testing, sample isolation for the detection of mRNA still required a column-based approach. Thus, further work will involve optimizing the protocol for simplified mRNA-isolation procedures. In addition, heating represents an essential step in the current sample-processing protocol using HUDSON. Thus, the integration of POC heating devices using chemical (69) or electromagnetic (70) heating might facilitate handling for the primary care provider or patient. Finally, inclusion of more patient samples and prospective analysis will allow for systematic comparison with current clinical practice. While this study focused on cellular-mediated rejection, the most frequent rejection affecting kidney-transplant recipients, next steps will include expanding and validating the rejection assay for the detection of antibody-mediated rejection and its potential complementary role to DSA testing (71, 72).

In summary, this work shows the application of CRISPR-Cas13 for the detection of rejection and opportunistic infection in kidney transplantation. This technology could be applied to other solid-organ transplants as well as immune-mediated kidney diseases such as lupus nephritis. On the basis of its low cost, ease of use and speed, this assay could enable frequent testing and earlier diagnosis. The next steps in order to advance clinical implementation include studies to validate these findings and to demonstrate the clinical utility of this assay in regard to long-term outcomes of kidney-transplant recipients.

### REFERENCES

1. Matas A J, Smith J M, Skeans M A, Thompson B, Gustafson S K, Stewart D E et al. OPTN/SRTR 2013 Annual Data Report: kidney. Am J Transplant Off J Am Soc Transplant Am Soc Transpl Surg. 2015 January; 15 Suppl 2:1-34.
2. Israni A K, Zaun D, Bolch C, Rosendale J D, Schaffhausen C, Snyder J J, et al. OPTN/SRTR 2015 Annual Data Report: Deceased Organ Donation. Am J Transplant. 2017; 17:503-42.
3. Lamb K E, Lodhi S, Meier-Kriesche H U. Long-term renal allograft survival in the United States: A critical reappraisal. Am J Transplant. 2011; 11(3):450-62.
4. El-Zoghby Z M, Stegall M D, Lager D J, Kremers W K, Amer H, Gloor J M, et al. Identifying specific causes of kidney allograft loss. Am J Transplant. 2009; 9(3):527-35.
5. Hu H, Aizenstein B D, Puchalski A, Burmania J A, Hamawy M M, Knechtle S J. Elevation of CXCR3-Binding Chemokines in Urine Indicates Acute Renal-Allograft Dysfunction. Am J Transplant. 2004; 4(3):432-7.
6. Schaub S, Nickerson P, Hess C, Mayr M, Rush D, Golian M, et al. Urinary CXCL9 and CXCL10 levels correlate with the extent of subclinical tubulitis. Am J Transplant. 2009; 9(6):1347-53.
7. Hricik D E, Nickerson P, Heeger P S, Bridges N D, Formica R N, Spain K, et al. Multicenter Validation of Urinary CXCL9 as a Risk-Stratifying Biomarker for Kidney Transplant Injury. Am J Transplant. 2013; 13(10):2634-44.
8. Hricik D E, Poggio E D, Armstrong B, Heeger P S, Schroppel B, Diop H, et al. Adverse Outcomes of Tacrolimus Withdrawal in Immune-Quiescent Kidney Transplant Recipients. J Am Soc Nephrol. 2015; 26(12):3114-22.
9. Matz M, Pratschke J, Seiler M, Babel N, Volk H-D, Reinke P, et al. Early post-transplant urinary IP-10 expression after kidney transplantation is predictive of short- and long-term graft function. Kidney Int [Internet]. 2006; 69(9):1683-90. Available from: http://dx.doi.org/10.1038/sj.ki.5000343
10. Jackson J A, Kim E J, Begley B, Cheeseman J, Harden T, Perez S D, et al. Urinary chemokines CXCL9 and CXCL10 are noninvasive markers of renal allograft rejection and B K viral infection. Am J Transplant. 2011; 11(10):2228-34.
11. Ho J, Nickerson P W, Bestland J, Gibson I W, Karpinski M, Rush D N, et al. Validation of Urinary CXCL10 As a Marker of Borderline, Subclinical, and Clinical Tubulitis.

Transplantation. 2011; 92(8):878-82.
12. Hirt-Minkowski P, Amico P, Ho J, Gao A, Bestland J, Hopfer H, et al. Detection of clinical and subclinical tubulo-interstitial inflammation by the urinary CXCL10 chemokine in a real-life setting. Am J Transplant. 2012; 12(7):1811-23.
13. Rabant M, Amrouche L, Timsit M O, Sauvaget V, Aulagnon F, Anglicheau D, et al. Urinary C-X-C Motif Chemokine 10 Independently Improves the Noninvasive Diagnosis of Antibody-Mediated Kidney Allograft Rejection. J Am Soc Nephrol. 2015; 26(11):2840-51.
14. Gootenberg J S, Collins J J, Lee J W, Bhattacharyya R P, Freije C A, Abudayyeh 00, et al. Nucleic acid detection with CRISPR-Cas13a/C2c2. Science (80-). 2017; 356(6336):438-42.
15. Gootenberg J S, Abudayyeh 00, Kellner M J, Joung J, Collins J J Z F. Multiplexed and portable nucleic acid detection platform with Cas13, Cas12a, and Csm6. 2016; 44(6387):61-70.
16. Chen J S, Ma E, Harrington L B, Da Costa M, Tian X, Palefsky J M et al. CRISPR-Cas12a target binding unleashes indiscriminate single-stranded DNase activity. Science. 2018; 360(6387):436-9.
17. Myhrvold C, Freije C A, Gootenberg J S, Abudayyeh 00, Metsky H C, Durbin A F et al. Field-deployable viral diagnostics using CRISPR-Cas13. Science (80-). 2018; 360(6387):444-8.
18. Henderson L K, Nankivell B J, Chapman J R. Surveillance protocol kidney transplant biopsies: Their evolving role in clinical practice. Am J Transplant. 2011; 11(8):1570-5.
19. Mehta R, Cherikh W, Sood P, Hariharan S. Kidney allograft surveillance biopsy practices across US transplant centers: A UNOS survey. Clin Transplant. 2017 May; 31(5).
20. Charo I F, Ransohoff R M. The Many Roles of Chemokines and Chemokine Receptors in Inflammation. N Engl J Med. 2006; 354(6):610-21.
21. Bromley S K, Mempel T R, Luster A D. Orchestrating the orchestrators: Chemokines in control of T cell traffic. Nat Immunol. 2008; 9(9):970-80.
22. Hancock W W, Gao W, Csizmadia V, Faia K L, Shemmeri N, Luster A D. Donor-derived IP-10 initiates development of acute allograft rejection. J Exp Med. 2001; 193(8):975-80.
23. Miura M, Morita K, Kobayashi H, Hamilton T A, Burdick M D, Strieter R M F R. Monokine Induced by IFN-Is a Dominant Factor Directing T Cells into Murine Cardiac Allografts During Acute Rejection. J Immunol. 2014; 167(6):3494-504.
24. Blydt-Hansen T D, Gibson I W, Gao A, Dufault B, Ho J. Elevated urinary CXCL10-to-creatinine ratio is associated with subclinical and clinical rejection in pediatric renal transplantation. Transplantation. 2015; 99(4):797-804.
25. Panzer U. Compartment-Specific Expression and Function of the Chemokine IP-10/CXCL10 in a Model of Renal Endothelial Microvascular Injury. J Am Soc Nephrol. 2005; 17(2):454-64.
26. Ingeborg A. Hauser, Sandra Spiegler, Eva Kiss, Stefan Gauer, Olaf Sichler, Ernst H. Scheuermann, Hanns Ackermann, Josef M. Pfeilschifter, Helmut Geiger, Hermann-Josef Grone HHR. Prediction of Acute Renal Allograft Rejection by Urinary Monokine Induced by IFN-gama (MIG). J Am Soc Nephrol. 2005; 16(6):1849-58.
27. Ho J, Schaub S, Wiebe C, Gao A, Wehmeier C, Koller M T, et al. Urinary CXCL10 Chemokine Is Associated with Alloimmune and Virus Compartment-Specific Renal Allograft Inflammation. Transplantation. 2018; 102(3):521-9.
28. Rabant M, Amrouche L, Morin L, Bonifay R, Lebreton X, Aouni L, et al. Early Low Urinary CXCL9 and CXCL10 Might Predict Immunological Quiescence in Clinically and Histologically Stable Kidney Recipients. Am J Transplant. 2016; 16(6):1868-81.
29. Hirsch H H, Randhawa P. B K polyomavirus in solid organ transplantation. Am J Transplant. 2013; 13(SUPPL.4):179-88.
30. Hirsch H H, Brennan D C, Drachenberg C B, Ginevri F, Gordon J, Limaye A P, et al. Polyomavirus-associated nephropathy in renal transplantation: Interdisciplinary analyses and recommendations. Transplantation. 2005; 79(10):1277-86.
31. Drachenberg C B, Papadimitriou J C, Chaudhry M R, Ugarte R, Mavanur M, Thomas B, et al. Histological Evolution of B K Virus-Associated Nephropathy: Importance of Integrating Clinical and Pathological Findings. Am J Transplant. 2017; 17(8):2078-91.
32. Keslar K S, Ding R, Bhasin M, Heeger P S, Rao P, Iklé D N, et al. Multicenter evaluation of a standardized protocol for noninvasive gene expression profiling. Am J Transplant. 2013; 13(7):1891-7.
33. Uffing A, Perez-Saez M J, La Manna G, Comai G, Fischman C, Farouk S, et al. A large, international study on post-transplant glomerular diseases: the TANGO project.

BMC Nephrol. 2018 September; 19(1):229.
34. Myhrvold C, Freije C A, Gootenberg J S, Abudayyeh 00, Metsky H C, Durbin A F, et al. Field-deployable viral diagnostics using CRISPR-Cas13. Science. 2018 April; 360(6387):444-8.
35. Suthanthiran M, Schwartz J E, Ding R, Abecassis M, Dadhania D, Samstein B, et al. Urinary-cell MRNA profile and acute cellular rejection in kidney allografts. J Urol. 2013; 190(6):2175-6.
36. De Vlaminck I, Valantine H A, Snyder T M, Strehl C, Cohen G, Luikart H, et al. Circulating cell-free DNA enables noninvasive diagnosis of heart transplant rejection. Sci Transl Med. 2014 June; 6(241):241ra77.
37. Bloom R D, Bromberg J S, Poggio E D, Bunnapradist S, Langone A J, Sood P, et al. Cell-Free DNA and Active Rejection in Kidney Allografts. J Am Soc Nephrol. 2017 July; 28(7):2221-32.
38. Haas M, Loupy A, Lefaucheur C, Roufosse C, Glotz D, Seron D, et al. The Banff 2017 Kidney Meeting Report: Revised diagnostic criteria for chronic active T cell-mediated rejection, antibody-mediated rejection, and prospects for integrative endpoints for next-generation clinical trials. Am J Transplant. 2018; 18(2):293-307.
39. Bujang M A, Adnan T H. Requirements for Minimum Sample Size for Sensitivity and Specificity Analysis. J Clin Diagn Res. 2016 October; 10(10):YE01-YE06.
40. J M B, D G A. Statistical methods for assessing agreement between two methods of clinical measurement. Lancet Lond Engl. 1986 Feb. 8; 1(8476):307-10.
41. Westgard J O, Barry P L, Hunt M R, Groth T: A multi-rule Shewhart chart for quality control in clinical chemistry. Clinical chemistry, 27: 493-501, 1981. 1981; 27(3):493-501.
42. Westgard J O, Ehrmeyer S S, Darcy T P, Westgard Quality Corporation. CLIA final rules for quality systems: quality assessment issues and answers. Madison, Wis., Westgard QC, 2004.
43. Arthur R R, Dagostin S, Shah K V. Detection of BK virus and J C virus in urine and brain tissue by the polymerase chain reaction. J Clin Microbiol. 1989; 27(6):1174-9.
44. Markowitz R B, Eaton B A, Kubik M F, Latorra D, McGregor J A, Dynan W S. B K virus and J C virus shed during pregnancy have predominantly archetypal regulatory regions. J Virol. 1991; 65(8):4515-9.
45. Jin L, Gibson P E, Booth J C, Clewley J P. Genomic typing of BK virus in clinical specimens by direct sequencing of polymerase chain reaction products. J Med Virol. 1993 September; 41(1):11-7. PubMed PMID: 8228931.
46. Bergallo M, Astegiano S, Sidoti F, Mantovani S, Segoloni G P, Cavallo R, et al. Real-time RT-PCR assay for the quantitation of polyomavirus BK VP1 mRNA levels in urine. Mol Biotechnol. 2010; 45(1):82-6.
47. Ding R, Medeiros M, Dadhania D, Muthukumar T, Kracker D, Kong J M, Epstein S R, Sharma V K, Seshan S V, Li B S M. Noninvasive diagnosis of BK virus nephritis by measurement of messenger RNA for BK virus VP1 in urine. Transplantation. 2002 Oct. 15; 74(7):987-94.
48. Tavis J E, Walker D L, Gardner S D, Frisque R J. Nucleotide sequence of the human polyomavirus AS virus, an antigenic variant of BK virus. J Virol. 1989 February; 63(2):901-11.
49. Anglicheau D, Naesens M, Gwinner W, Marquet P, Essig M. Establishing Biomarkers in Transplant Medicine. Transplantation. 2016; 100(10):2024-38.
50. Lo D J, Kaplan B, Kirk A D. Biomarkers for kidney transplant rejection. Nat Rev Nephrol [Internet]. 2014; 10(4):215-25.
51. Wiebe C, Rush D N, Nevins T E, Birk P E, Blydt-Hansen T, Gibson I W, et al. Class II Eplet Mismatch Modulates Tacrolimus Trough Levels Required to Prevent Donor-Specific Antibody Development. J Am Soc Nephrol. 2017 November; 28(11):3353-62.
52. Galichon P, Amrouche L, Hertig A, Brocheriou I, Rabant M, Xu-Dubois Y C, et al. Urinary mRNA for the diagnosis of renal allograft rejection: The issue of normalization. Am J Transplant. 2016; 16(10):3033-40.
53. Sepkowitz, K. A. Opportunistic Infections in Patients with and Patients without Acquired Immunodeficiency Syndrome. Clin. Infect. Dis. 34, 1098-1107 (2002).
54. Harrington, L. B. et al. CRISPR-Cas12a target binding unleashes indiscriminate single-stranded DNase activity. Science (80-.). 360, 436-439 (2018).
55. Hajian, R. et al. Detection of unamplified target genes via CRISPR-Cas9 immobilized on a graphene field-effect transistor. Nat. Biomed. Eng. 3, (2019).
56. Harrington, L. B. et al. Programmed DNA destruction by miniature CRISPR-Cas14 enzymes. Science 362, 839-842 (2018).
57. Pardee, K. et al. Rapid, low-cost detection of Zika virus using programmable biomolecular components. Cell 165, 1255-1266 (2016).
58. Haijan, R. et al. Detection of unamplified target genes via CRISPR-Cas9 immobilized on a graphene field-effect transistor. Nat. Biomed. Eng. 3, 427-437 (2019).
59. Wu, Y., Liu, S.-X., Wang, F. & Zeng, M.-S. Room temperature detection of plasma Epstein-Barr virus DNA with CRISPR-Cas13. Clin. Chem. 65, 591-592 (2019).
60. Tullius, S. G. & Rabb, H. Improving the supply and quality of deceased-donor organs for transplantation. N. Engl. J. Med. 378, 1920-1929 (2018).
61. Wekerle, T., Segev, D., Lechler, R. & Oberbauer, R. Strategies for long-term preservation of kidney graft function. Lancet 389, 2152-2162 (2017).
62. Ross, S. A., Novak, Z., Pati, S. & Boppana, S. B. Overview of the diagnosis of cytomegalovirus infection. Infect. Disord. Drug Targets 11, 466-474 (2011).
63. Randhawa, P. et al. Correlates of quantitative measurement of B K polyomavirus (BKV) DNA with clinical course of BKV infection in renal transplant patients. J.

Clin. Microbiol. 42, 1176-1180 (2004).
64. Fishman, J. A. Infection in organ transplantation. Am. J. Transplant. 17, 856-879 (2017).
65. Sepkowitz, K. A. Opportunistic infections in patients with and patients without acquired immunodeficiency syndrome. Clin. Infect. Dis. 34, 1098-1107 (2002).
66. Sundsfjord, A. et al. B K and J C viruses in human immunodeficiency virus type 1-infected persons: prevalence, excretion, viremia, and viral regulatory regions. J.

Infect. Dis. 169, 485-490 (1994).
67. Fishman, J. A. Infection in solid-organ transplant recipients. N. Engl. J. Med. 357, 2601-2614 (2007).
68. Sigdel, T. K. et al. Optimizing detection of kidney transplant injury by assessment of donor-derived cell-free DNA via massively multiplex PCR. J. Clin. Med. 8, E19 (2018).
69. Curtis, K. A. et al. Isothermal amplification using a chemical heating device for point-of-care detection of HIV-1. PLoS ONE 7, e31432 (2012).
70. Ahn, M.-H., Baek, S.-K., Min, J. & Park, J.-H. A portable electromagnetic induction heating device for point-of-care diagnostics. BioChip J. 10, 208-214 (2016).
71. Parajuli, S. et al. Donor-specific antibodies in the absence of rejection are not a risk factor for allograft failure. Kidney Int. Rep. 4, 1057-1065 (2019).
72. Schinstock, C. A. et al. The value of protocol biopsies to identify patients with de novo donor-specific antibody at high risk for allograft loss. Am. J. Transpl. 17, 1574-1584 (2017).
73. Katoh, K. & Standley, D. M. MAFFT multiple sequence alignment software version 7: improvements in performance and usability. Mol. Biol. Evol. 30, 772-780 (2013).
74. Waterhouse, A. M., Procter, J. B., Martin, D. M. A., Clamp, M. & Barton, G. J. Jalview Version 2—a multiple sequence alignment editor and analysis workbench. Bioinformatics 25, 1189-1191 (2009).
75. Schmittgen, T. D. & Livak, K. J. Analyzing real-time PCR data by the comparative C T method. Nat. Protoc. 3, 1101 (2008).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A nucleic acid detection system comprising:
a CRISPR component comprising: (i) an effector protein; and (ii) a guide RNA, and/or a polynucleotide encoding a guide RNA, that binds or hybridizes to a corresponding target molecule;
wherein the target molecule is a polynucleic acid that is indicative of rejection and/or infection of an organ transplant.

2. The detection system of claim 1, wherein the effector protein is Cas13.

3. The detection system of claim 1 or claim 2, wherein the target molecule is selected from the group consisting of viral DNA and cytokine mRNA.

4. The detection system of any one of claims 1-3, wherein the target molecule is selected from the group consisting of BK polyomavirus DNA, cytomegalovirus DNA, CXCL9 mRNA, CXCL10 mRNA, and a combination thereof.

5. The detection system of any one of claims 1-4, wherein the detection system comprises:
a guide RNA comprising the nucleic acid sequence of TTGCTACTGCATTGACTGCTTCACACAG (SEQ ID NO: 20);
a guide RNA comprising the nucleic acid sequence of ACGAGGTCCGTGTGGATCCGCTGACGCG (SEQ ID NO: 21);
a guide RNA comprising the nucleic acid sequence of ATGATTTCAATTTTCTCGCAGGAAGGGC (SEQ ID NO: 22); or
a combination thereof.

6. The detection system of any one of claims 1-5, wherein the detection system comprises:
a guide RNA comprising the nucleic acid sequence of GATTTAGACTACCCCAAAAACGAAGGGGACTAAAACTTGCTACTGCATTGACTG CTTCACACAG (SEQ ID NO: 17);
a guide RNA comprising the nucleic acid sequence of GATTTAGACTACCCCAAAAACGAAGGGGACTAAAACACGAGGTCCGTGTGGATC CGCTGACGCG (SEQ ID NO: 18);
a guide RNA comprising the nucleic acid sequence of GATTTAGACTACCCCAAAAACGAAGGGGACTAAAACATGATTTCAATTTTCTCGC AGGAAGGGC (SEQ ID NO: 19); or
a combination thereof.

7. The detection system of any one of claims 1-6, wherein the detection system comprises:
a polynucleotide encoding a guide RNA and comprising the nucleic acid sequence of CTGTGTGAAGCAGTCAATGCAGTAGCAAGTTTTAGTCCCCTTCGTTTTTGGGGTA GTCTAAATCCCTATAGTGAGTCGTATTAATTTC (SEQ ID NO: 14);
a polynucleotide encoding a guide RNA and comprising the nucleic acid sequence of CGCGTCAGCGGATCCACACGGACCTCGTGTTTTAGTCCCCTTCGTTTTTGGGGTA GTCTAAATCCCTATAGTGAGTCGTATTAATTTC (SEQ ID NO: 15);
a polynucleotide encoding a guide RNA and comprising the nucleic acid sequence of GCCCTTCCTGCGAGAAAATTGAAATCATGTTTTAGTCCCCTTCGTTTTTGGGGTAG TCTAAATCCCTATAGTGAGTCGTATTAATTTC (SEQ ID NO: 16); or
a combination thereof.

8. The detection system of any one of claims 1-7, further comprising an amplification component, wherein the amplification component comprises a polymerase and one or more primer.

9. The detection system of claim 8, wherein amplification component comprises a DNA polymerase, an RNA polymerase, or a combination thereof.

10. The detection system of claim 8 or claim 9, wherein the amplification component comprises an RNA polymerase, wherein the RNA polymerase is T7 RNA polymerase.

11. The detection system of any one of claims 8-10, wherein the detection system comprises a forward primer and a reverse primer, wherein the forward and reverse primer concentrations are 120 nM and 480 nM, respectively.

12. The detection system of any one of claims 8-11, wherein the detection system comprises a forward primer and a reverse primer, wherein the reverse primer comprises an RNA polymerase promoter sequence.

13. The detection system of claim 12, wherein the RNA polymerase promoter sequence is a T7 polymerase promoter sequence.

14. The detection system of claim 13, wherein the T7 polymerase promoter sequence comprise the nucleic acid sequence of GAAATTAATACGACTCACTATAGG (SEQ ID NO: 13).

15. The detection system of any one of claims 8-14, wherein the detection system comprises:
a forward primer comprising the nucleic acid sequence of CATTGCAGAGTTTCTTCAGTTAGGTCTAAGCC (SEQ ID NO: 25); and
a reverse primer comprising the nucleic acid sequence of
(SEQ ID NO: 2)
AATTTTTAAGAAAAGAGCCCTTGGTTTGGATA.

16. The detection system of claim 15, wherein the forward primer comprises the nucleic acid sequence of
(SEQ ID NO: 1)
GAAATTAATACGACTCACTATAGGCATTGCAGAGTTTCTTCAGTTAG
GTCTAAGCC.

17. The detection system of any one of claims 8-16, wherein the detection system comprises:
a forward primer comprising the nucleic acid sequence of GCACCAGCCGAACGTGGTGATCCGCCGATCGATGAC (SEQ ID NO: 26); and
a reverse primer comprising the nucleic acid sequence of
(SEQ ID NO: 4)
CTATCAGCAACTGGACCATGGCCAGAAAAATCG.

18. The detection system of claim 17, wherein the forward primer comprises the nucleic acid sequence of
(SEQ ID NO: 3)
GAAATTAATACGACTCACTATAGGGCACCAGCCGAACGTGGTGATCC
GCCGATCGATGAC.

19. The detection system of any one of claims 8-18, wherein the detection system comprises:
a forward primer comprising the nucleic acid sequence of TATCCACCTACAATCCTTGAAAGACCTTAAAC (SEQ ID NO: 27); and
a reverse primer comprising the nucleic acid sequence of
(SEQ ID NO: 6)
TTAGACATGTTTGAACTCCATTCTTCAGTGTA.

20. The detection system of claim 19, wherein the forward primer comprises the nucleic acid sequence of
(SEQ ID NO: 5)
GAAATTAATACGACTCACTATAGGTATCCACCTACAATCCTTGAAAG
ACCTTAAAC.

21. The detection system of any one of claims 1-20, further comprising an RNase inhibitor.

22. The detection system of any one of claims 1-21, further comprising an oligonucleotide comprising a detectable molecule that is detectable when the oligonucleotide is cleaved by the effector protein.

23. The detection system of claim 22, wherein the detectable molecule is a quenched fluorophore that exhibits fluorescence when the oligonucleotide is cleaved by the effector protein.

24. The detection system of claim 22, wherein the detectable molecule is a lateral flow reporter molecule comprising the nucleic acid sequence of 6FAM-mArArUrGrGrCmAmArArUrGrGrCmA-BIO (SEQ ID NO: 24).

25. The detection system of any one of claims 1-24 further comprising one or more reaction buffers.

26. A diagnostic device comprising: (i) one or more compartments comprising a detection system of any one of claims 1-25; and (ii) one or more substrates.

27. The diagnostic device of claim 26, wherein the substrate is a lateral flow strip, a flexible material substrate, a paper substrate, or a flexible polymer-based substrate.

28. The diagnostic device of claim 26 or claim 27, wherein the diagnostic device further comprises an imaging component.

29. The diagnostic device of claim 28, wherein the diagnostic device further comprises an imaging analysis component.

30. The diagnostic device of claim 29, wherein the imaging analysis component comprises a lateral-flow quantification application.

31. The diagnostic device of any one of claims 26-30, wherein the device comprises a compartment comprising a detection system and a polynucleotide positive control, wherein the polynucleotide positive control comprises a nucleic acid sequence that is bound or hybridized by a guide RNA of the detection system.

32. The diagnostic device of claim 31, wherein the polynucleotide positive control comprises the nucleic acid sequence of
(SEQ ID NO: 28)
ATGAAGAAAAGTGGTGTTCTTTTCCTCTTGGGCATCATCTTGCTGGT
TCTGATTGGAGTGCAAGGAACCCCAGTAGTGAGAAAGGGTCGCTGTT
CCTGCATCAGCACCAACCAAGGGACTATCCACCTACAATCCTTGAAA
GACCTTAAACAATTTGCCCCAAGCCCTTCCTGCGAGAAAATTGAAAT
CATTGCTACACTGAAGAATGGAGTTCAAACATGTCTAAACCCAGATT
CAGCAGATGTGAAGGAACTGATTAAAAAGTGGGAGAAACAGGTCAGC
CAAAAGAAAAAGCAAAAGAATGGGAAAAAACATCAAAAAAAGAAAGT
TCTGAAAGTTCGAAAATCTCAACGTTCTCGTCAAAAGAAGACTACAT
AA.

33. The diagnostic device of claim 31 or claim 32, wherein the polynucleotide positive control comprises the nucleic acid sequence of
(SEQ ID NO: 23)
GAAATTAATACGACTCACTATAGGATGAAGAAAAGTGGTGTTCTTTT
CCTCTTGGGCATCATCTTGCTGGTTCTGATTGGAGTGCAAGGAACCC
CAGTAGTGAGAAAGGGTCGCTGTTCCTGCATCAGCACCAACCAAGGG
ACTATCCACCTACAATCCTTGAAAGACCTTAAACAATTTGCCCCAAG
CCCTTCCTGCGAGAAAATTGAAATCATTGCTACACTGAAGAATGGAG
TTCAAACATGTCTAAACCCAGATTCAGCAGATGTGAAGGAACTGATT
AAAAAGTGGGAGAAACAGGTCAGCCAAAAGAAAAAGCAAAAGAATGG
GAAAAAACATCAAAAAAAGAAAGTTCTGAAAGTTCGAAAATCTCAAC
GTTCTCGTCAAAAGAAGACTACATAA.

34. The diagnostic device of any one of claims 26-33, wherein the device comprises a compartment comprising a detection system and a polynucleotide negative control, wherein the polynucleotide negative control comprises a nucleic acid sequence that is not bound or hybridized by a guide RNA of the detection system.

35. A kit comprising a detection system of any one of claims 1-25 or a diagnostic device of any one of claims 26-34.

36. The kit of claim 35, further comprising a polynucleic acid isolation component.

37. The kit of claim 36, wherein the polynucleic acid isolation component comprises tris(2-carboxyethyl)phosphine, EDTA, or a combination thereof.

38. The kit of claim 35 or 36, wherein the polynucleic acid isolation component comprises a purification column.

39. The kit of any one of claims 35-38, wherein the kit comprises a polynucleotide positive control, wherein the polynucleotide positive control comprises a nucleic acid sequence that is bound or hybridized by a guide RNA of the detection system.

40. The kit of claim 39, wherein the polynucleotide positive control comprises the nucleic acid sequence of
(SEQ ID NO: 28)
ATGAAGAAAAGTGGTGTTCTTTTCCTCTTGGGCATCATCTTGCTGGT
TCTGATTGGAGTGCAAGGAACCCCAGTAGTGAGAAAGGGTCGCTGTT
CCTGCATCAGCACCAACCAAGGGACTATCCACCTACAATCCTTGAAA
GACCTTAAACAATTTGCCCCAAGCCCTTCCTGCGAGAAAATTGAAAT
CATTGCTACACTGAAGAATGGAGTTCAAACATGTCTAAACCCAGATT
CAGCAGATGTGAAGGAACTGATTAAAAAGTGGGAGAAACAGGTCAGC
CAAAAGAAAAAGCAAAAGAATGGGAAAAAACATCAAAAAAAGAAAGT
TCTGAAAGTTCGAAAATCTCAACGTTCTCGTCAAAAGAAGACTACAT
AA.

41. The kit of claim 38 or claim 39, wherein the polynucleotide positive control comprises the nucleic acid sequence of
(SEQ ID NO: 23)
GAAATTAATACGACTCACTATAGGATGAAGAAAAGTGGTGTTCTTTT
CCTCTTGGGCATCATCTTGCTGGTTCTGATTGGAGTGCAAGGAACCC
CAGTAGTGAGAAAGGGTCGCTGTTCCTGCATCAGCACCAACCAAGGG
ACTATCCACCTACAATCCTTGAAAGACCTTAAACAATTTGCCCCAAG
CCCTTCCTGCGAGAAAATTGAAATCATTGCTACACTGAAGAATGGAG
TTCAAACATGTCTAAACCCAGATTCAGCAGATGTGAAGGAACTGATT
AAAAAGTGGGAGAAACAGGTCAGCCAAAAGAAAAAGCAAAAGAATGG
GAAAAAACATCAAAAAAAGAAAGTTCTGAAAGTTCGAAAATCTCAAC
GTTCTCGTCAAAAGAAGACTACATAA.

42. The kit of any one of claims 35-41, wherein the kit comprises a compartment comprising a detection system and a polynucleotide negative control, wherein the polynucleotide negative control comprises a nucleic acid sequence that is not bound or hybridized by a guide RNA of the detection system.

43. A method for detecting a target molecule in a sample, comprising contacting the sample with the detection system of any one of claims 1-25, the device of any one of claims 26-34, or the kit of any one of claims 35-42.

44. The method of claim 43, wherein the sample is a urine sample, a blood sample, a serum sample, or a plasma sample from a patient having an organ transplant.

45. The method of claim 44, wherein the organ transplant is a renal transplant.

46. The method of any one of claims 43-45, wherein the method comprises purifying polynucleotides from the sample.

47. The method of any one of claims 43-46, wherein the method comprises amplifying the target molecule using an isothermal recombinase polymerase amplification method (RPA).

48. The method of any one of claims 43-47, wherein RNases in the sample are inhibited.

49. A method of detecting an opportunistic post-transplantation viral infection comprising:
contacting nucleic acids from a sample obtained from a transplant patient with a detection system of any one of claims 1-25, a device of any one of claims 26-34, or a kit of any one of claims 35-42;
wherein the target molecule is a polynucleic acid that is indicative of an opportunistic post-translational viral infection.

50. The method of claim 49, wherein the target is a viral DNA molecule.

51. The method of claim 49 or claim 50, wherein the target molecule is selected from the group consisting of BK polyomavirus DNA, cytomegalovirus DNA, or a combination thereof.

52. The method of any one of claims 49-51, wherein the sample is a urine sample, a blood sample, a serum sample, or a plasma sample from a patient having an organ transplant.

53. The method of claim 52, wherein the organ transplant is a renal transplant.

54. The method of any one of claims 49-53, wherein the method comprises purifying polynucleotides from the sample.

55. The method of any one of claims 49-54, wherein the method comprises amplifying the target molecule using an isothermal recombinase polymerase amplification method (RPA).

56. The method of any one of claims 49-55, wherein RNases in the sample are inhibited.

57. A method for identifying a subject having BK nephropathy comprising:
contacting nucleic acids from a sample obtained from a patient with a detection system of any one of claims 1-25, a device of any one of claims 26-34, or a kit of any one of claims 35-42;
wherein the target molecule is a polynucleic acid that is indicative of BK nephropathy.

58. The method of claim 57, wherein the target is a viral DNA molecule.

59. The method of claim 57, wherein the target is BK polyomavirus DNA.

60. The method of any one of claims 57-59, wherein the method comprises purifying polynucleotides from the sample.

61. The method of any one of claims 57-60, wherein the method comprises amplifying the target molecule using an isothermal recombinase polymerase amplification method (RPA).

62. The method of any one of claims 57-61, wherein RNases in the sample are inhibited.

63. A method for the monitoring of transplant rejection comprising:
contacting nucleic acids from a sample obtained from a transplant patient with a detection system of any one of claims 1-25, a device of any one of claims 26-34, or a kit of any one of claims 35-42;
wherein the target molecule is a polynucleic acid that is indicative of transplant rejection.

64. The method of claim 63, wherein the target molecule is a cytokine mRNA.

65. The method of claim 63 or claim 64, wherein the target molecule is a CXCL9 mRNA, CXCL10 mRNA, and a combination thereof.

66. The method of any one of claims 63-65, wherein the sample is a urine sample, a blood sample, a serum sample, or a plasma sample from a patient having an organ transplant.

67. The method of claim 66, wherein the organ transplant is a renal transplant.

68. The method of any one of claims 63-67, wherein the method comprises purifying polynucleotides from the sample.

69. The method of any one of claims 63-68, wherein the method comprises amplifying the target molecule using an isothermal recombinase polymerase amplification method (RPA).

70. The method of any one of claims 63-69, wherein RNases in the sample are inhibited.
